SEQUENCE LISTING (205 sequences)

SEQ ID NO: 1 - protein, 114 aa, synthetic construct
EVQLVQSGGGLVQPGGSLRLSCAASGIFFSDTHMGWYRQAPGKERELVAIITSGGSTDYVDSVKGRFTISRDNAKNTVYLQMNTLKFEDTAVYYCNARRTTIYWGQGTQVTVSS

SEQ ID NO: 2 - protein, 114 aa, synthetic construct
QVQLVESGGGLVQPGGSLRLSCAASGIDFSDTHMGWYRQAPGKERELVALITSGGFTDYADSVKGRFTISRDNAKNTMYLQMNTLKFEDTAVYYCNARRTTIYWGQGTQVTVSS

SEQ ID NO: 3 - protein, 114 aa, synthetic construct
EVQLVESGGGLVQPGGSLRLSCVASGFDFSDTHMGWYRQAPGKQRELVALITSGAFTDYADSVKGRFTISRDNAKNTVYLQMNTLKFEDTAVYYCNARRTTIYWGQGTQVTVSS

SEQ ID NO: 4 - protein, 114 aa, synthetic construct
EVQLVESGGGLVQPGGSLRLSCAASGIDFSDTHMGWYRQAPGKQRELVALITSGGFTDYADSVKGRFTISRDNAKNTMYLQMNTLKLEDTAVYYCNARRTTIYWGQGTQVTVSS

SEQ ID NO: 5 - protein, 114 aa, synthetic construct
QVQLVESGGGLVQPGGSLRLSCAASGIFFSDTHMGWYRQAPGKERELVAIITSGGSTDYVDSVKGRFTISRDNAKNTVYLQMNSLKFEDTAVYYCNARRTTIYWGQGTQVTVSS

SEQ ID NO: 6 - protein, 114 aa, synthetic construct
QVQLVESGGGLVQPGGSLRLSCAASGIDFSDTHMGWYRQAPGKERELVALITSGGFTDYADSVKGRFTISRDNAKNTMYLQMNTLKFEDTAVYYCNARKTTIYWGQGTQVTVSS

SEQ ID NO: 7 - protein, 114 aa, synthetic construct
EVQLVESGGGLVQPGGSLRLSCGASGIFFSGVHMGWYRQAPGKERELVALITDSGSADYVDSVKGRFTISRDNAKNTVYLQMNSLKFEDTAVYYCNARTTTIYWGQGTQVTVSS

SEQ ID NO: 8 - protein, 114 aa, synthetic construct
QVQLVESGGGLVQPGGSLRLSCAASGIDFSDTHMGWYRQAPGKERELVALITSGGFTDYADSVKGRFTISRDNAKNTMYLQMNTLKFEDTAVYYCNARRSTIYWGQGTQVTVSS

SEQ ID NO: 9 - protein, 118 aa, synthetic construct
QVQLVESGGGLVQAGGSLRLSCAASGFSASDVVMAWYRQAPGKQREFVANIAARGSTDYANSVKGRFTISRDNAKNTVYLQMNSLKSDDTAVYYCNALVWNGSSYNNWGQGTQVTVSS

SEQ ID NO: 10 - protein, 118 aa, synthetic construct
QVQLVESGGGLVQAGGSLRLSCAASGFSASDVVMAWYRQAPGKQREFVANIAASSTTRYADSVKGRFTISRDDAKNTVSLQMNSLKSEDTAVYYCNALVWNGSSYNNWGQGTQVTVSS

SEQ ID NO: 11 - protein, 118 aa, synthetic construct
QVQLVESGGGLVQAGGSLRLSCAASGFSASDVVMAWYRQAPGKQREFVANLGASSTTRYADSVKGRFTISRDDAKNTVSLQMNSLKSEDTAVYYCNALVWNGSSYNNWGQGTQVTVSS

SEQ ID NO: 12 - protein, 118 aa, synthetic construct
EVQLVESGGGSVQDGGSLTLSCAASGFSAGDVVMAWYRQAPGKQREFVANIAASSTTRYTDSVKGRFTISRDNAKNTVYLQMNSLKSEDTAVYYCNALVWNGSSYNNWGQGTQVTVSS

SEQ ID NO: 13 - protein, 114 aa, synthetic construct
QLQLVESGGGVVQPGGSLRLSCAASGIDFSDTHMGWYRQAPGKQRELVAIITSGGSTDYVDSVKGRFTISRDNAKNTVYLQMNSLKFEDTAVYYCNARRSTIYWGQGTQVTVSS

SEQ ID NO: 14 - protein, 114 aa, synthetic construct
EVQLVESGGGLVQPGGSLRLSCVASGFDFSDTHMGWYRQAPGKERELVAIITSGGSTDYVDSVKGRFTISRDNAKNTVYLQMNSLKFEDTAVYYCNARRTTIYWGQGTQVTVSS

SEQ ID NO: 15 - protein, 114 aa, synthetic construct
QVQLVESGGDLVQPGGSLRLSCAASGIDFSDTHMGWYRQAPGKQRELVALITSGGFTDYPDSVKGRFTISRDNAKNTMYLQMNTLKLEDTAVYYCNARRTTIYWGQGTQVTVSS

SEQ ID NO: 16 - protein, 114 aa, synthetic construct; identical to SEQ ID NO: 14

SEQ ID NO: 17 - protein, 114 aa, synthetic construct
EVQLVESGGGLVQPGGSLRLSCAASGFDFSDTHMSWVRQAPGKERELVSVITSGGSTYYADSVKGRFTISRDNSKNTLYLQMNSLRAEDTAVYYCNARRTTIYWGQGTQVTVSS

SEQ ID NO: 18 - protein, 114 aa, synthetic construct
EVQLVESGGGLVQPGGSLRLSCAASGFDFSDTHMSWVRQAPGKERELVSAITSGGSTYYADSVKGRFTISRDNSKNTLYLQMNSLRAEDTAVYYCNARRTTIYWGQGTQVTVSS

SEQ ID NO: 19 - protein, 114 aa, synthetic construct
EVQLVESGGGLVQPGGSLRLSCAASGFDFSDTHMNWVRQAPGKERELVSYITSGGSTYYADSVKGRFTISRDNAKNSLYLQMNSLRAEDTAVYYCNARRTTIYWGQGTQVTVSS

SEQ ID NO: 20 - protein, 114 aa, synthetic construct
QVQLVESGGGLKQPGGSLRLSCAASGFDFSDTHMSWIRQAPGKERELVSYITSGGSTYYADSVKGRFTISRDNAKNSLYLQMNSLRAEDTAVYYCNARRTTIYWGQGTQVTVSS

SEQ ID NO: 21 - protein, 114 aa, synthetic construct
EVQLVESGGGLVQPGGSLRLSCAASGFDFSDTHMSWVRQAPGKERELVSVITSGGSTDYADSVKGRFTISRDNSKNTLYLQMNSLRAEDTAVYYCNARRTTIYWGQGTQVTVSS

SEQ ID NO: 22 - protein, 114 aa, synthetic construct
EVQLVESGGGLVQPGGSLRLSCAASGFDFSDTHMSWVRQAPGKERELVSVITSGGSTDYVDSVKGRFTISRDNSKNTLYLQMNSLRAEDTAVYYCNARRTTIYWGQGTQVTVSS

SEQ ID NO: 23 - protein, 114 aa, synthetic construct
EVQLVESGGGLVQPGGSLRLSCAASGFDFSDTHMSWVRQAPGKERELVSIITSGGSTDYVDSVKGRFTISRDNSKNTLYLQMNSLRAEDTAVYYCNARRTTIYWGQGTQVTVSS

SEQ ID NO: 24 - protein, 114 aa, synthetic construct
EVQLVESGGGLVQPGGSLRLSCAASGFDFSDTHMSWVRQAPGKERELVAIITSGGSTDYVDSVKGRFTISRDNSKNTLYLQMNSLRAEDTAVYYCNARRTTIYWGQGTQVTVSS

SEQ ID NO: 25 - protein, 114 aa, synthetic construct; identical to SEQ ID NO: 13

SEQ ID NO: 26 - protein, 114 aa, synthetic construct
EVQLVESGGGLVQPGGSLRLSCAASGIDFSDTHMSWVRQAPGKQRELVSVITSGGSTYYADSVKGRFTISRDNSKNTLYLQMNSLRAEDTAVYYCNARRSTIYWGQGTQVTVSS

SEQ ID NO: 27 - protein, 114 aa, synthetic construct
QVQLVESGGGVVQPGGSLRLSCAASGIDFSDTHMHWVRQAPGKQRELVSVITSGGSTYYADSVKGRFTISRDNSKNTLYLQMNSLRAEDTAVYYCNARRSTIYWGQGTQVTVSS

SEQ ID NO: 28 - protein, 114 aa, synthetic construct
QVQLVESGGGLVKPGGSLRLSCAASGIDFSDTHMSWIRQAPGKQRELVSYITSGGSTNYADSVKGRFTISRDNAKNSLYLQMNSLRAEDTAVYYCNARRSTIYWGQGTQVTVSS

SEQ ID NO: 29 - protein, 114 aa, synthetic construct
QVQLVESGGGVVQPGGSLRLSCAASGIDFSDTHMHWVRQAPGKQRELVSVITSGGSTDYADSVKGRFTISRDNSKNTLYLQMNSLRAEDTAVYYCNARRSTIYWGQGTQVTVSS

SEQ ID NO: 30 - protein, 114 aa, synthetic construct
QVQLVESGGGVVQPGGSLRLSCAASGIDFSDTHMHWVRQAPGKQRELVSVITSGGSTDYVDSVKGRFTISRDNSKNTLYLQMNSLRAEDTAVYYCNARRSTIYWGQGTQVTVSS

SEQ ID NO: 31 - protein, 114 aa, synthetic construct
QVQLVESGGGVVQPGGSLRLSCAASGIDFSDTHMHWVRQAPGKQRELVSIITSGGSTDYVDSVKGRFTISRDNSKNTLYLQMNSLRAEDTAVYYCNARRSTIYWGQGTQVTVSS

SEQ ID NO: 32 - protein, 114 aa, synthetic construct
QVQLVESGGGVVQPGGSLRLSCAASGIDFSDTHMHWVRQAPGKQRELVAIITSGGSTDYVDSVKGRFTISRDNSKNTLYLQMNSLRAEDTAVYYCNARRSTIYWGQGTQVTVSS

SEQ ID NO: 33 - protein, 114 aa, synthetic construct
QVQLVESGGGVVQPGGSLRLSCAASGIDFSDTHMHWVRQAPGKQRELVAIITSGGFTDYVDSVKGRFTISRDNSKNTLYLQMNSLRAEDTAVYYCNARTSTIYWGQGTQVTVSS

SEQ ID NO: 34 - protein, 114 aa, synthetic construct
QVQLVESGGGVVQPGGSLRLSCAASGIDFSDTHMHWVRQAPGKQRELVAIITSGGFTDYVDSVKGRFTISRDNSKNTLYLQMNSLRAEDTAVYYCNARRSTIYWGQGTQVTVSS

SEQ ID NO: 35 - protein, 114 aa, synthetic construct
QVQLVESGGGVVQPGGSLRLSCAASGIDFSDTHMHWVRQAPGKQRELVAIITSSGSTDYVDSVKGRFTISRDNSKNTLYLQMNSLRAEDTAVYYCNARRSTILWGQGTQVTVSS

SEQ ID NO: 36 - protein, 114 aa, synthetic construct
QVQLVESGGGVVQPGGSLRLSCAASGIDFDDTHMHWVRQAPGKQRELVAIITSGGLTDYVDSVKGRFTISRDNSKNTLYLQMNSLRAEDTAVYYCNARTSTIYWGQGTQVTVSS

SEQ ID NO: 37 - protein, 114 aa, synthetic construct
QVQLVESGGGVVQPGGSLRLSCAASGIDFNDTHMHWVRQAPGKQRELVAIITSRGSTDYVDSVKGRFTISRDNSKNTLYLQMNSLRAEDTAVYYCNARRSTLYWGQGTQVTVSS

SEQ ID NO: 38 - protein, 114 aa, synthetic construct
QVQLVESGGGVVQPGGSLRLSCAASGIDFSDLHMHWVRQAPGKQRELVAIITSGGSTDYVDSVKGRFTISRDNSKNTLYLQMNSLRAEDTAVYYCNARSSTIYWGQGTQVTVSS

SEQ ID NO: 39 - protein, 114 aa, synthetic construct
EVQLVESGGGLVQPGGSLRLSCAASGFDFSDTHMSWVRQAPGKERELVAIITGGGSTDYVDSVKGRFTISRDNSKNTLYLQMNSLRAEDTAVYYCLARRTTIYWGQGTQVTVSS

SEQ ID NO: 40 - protein, 114 aa, synthetic construct
EVQLVESGGGLVQPGGSLRLSCAASGFDFSDTHMSWVRQAPGKERELVAIITSGGSTDYVDSVKGRFTISRDNSKNTLYLQMNSLRAEDTAVYYCNARLTTIYWGQGTQVTVSS

SEQ ID NO: 41 - protein, 114 aa, synthetic construct
EVQLVESGGGLVQPGGSLRLSCAASGFDFSDTFMSWVRQAPGKERELVAIITSGGSTDYVDSVKGRFTISRDNSKNTLYLQMNSLRAEDTAVYYCNARRTTIYWGQGTQVTVSS

SEQ ID NO: 42 - protein, 114 aa, synthetic construct
EVQLVESGGGLVQPGGSLRLSCAASGFDFPDTHMSWVRQAPGKERELVAIITSGGFTDYVDSVKGRFTISRDNSKNTLYLQMNSLRAEDTAVYYCRARRTTIYWGQGTQVTVSS

SEQ ID NO: 43 - protein, 114 aa, synthetic construct
EVQLVESGGGLVQPGGSLRLSCAASGFDFSDTAMSWVRQAPGKERELVAIITSGGSVDYVDSVKGRFTISRDNSKNTLYLQMNSLRAEDTAVYYCRARRTTIYWGQGTQVTVSS

SEQ ID NO: 44 - protein, 114 aa, synthetic construct
EVQLVESGGGLVQPGGSLRLSCAASGFDFSMTHMSWVRQAPGKERELVAIITSGGKTDYVDSVKGRFTISRDNSKNTLYLQMNSLRAEDTAVYYCNARRATIYWGQGTQVTVSS

SEQ ID NO: 45 - protein, 114 aa, synthetic construct
EVQLVESGGGLVQPGGSLRLSCAASGFDFEDTHMSWVRQAPGKERELVAIITSGCSTDYVDSVKGRFTISRDNSKNTLYLQMNSLRAEDTAVYYCNARRATIYWGQGTQVTVSS

SEQ ID NO: 46 - protein, 118 aa, synthetic construct
QVQLVESGGGLVQAGGSLRLSCAASGFSASDVVMAWYRQAPGKQREFVANIAARGSTDYANSVKGRFTISRDNAKNTVYLQMNSLKSDDTAVYYCNALVWQGSSYNNWGQGTQVTVSS

SEQ ID NO: 47 - protein, 118 aa, synthetic construct
QVQLVESGGGLVQAGGSLRLSCAASGFSASDVVMAWYRQAPGKQREFVANIAARGSTDYANSVKGRFTISRDNAKNTVYLQMNSLKSDDTAVYYCNALVWTGSSYNNWGQGTQVTVSS

SEQ ID NO: 48 - protein, 118 aa, synthetic construct
QVQLVESGGGLVQAGGSLRLSCAASGFSASDVVMAWYRQAPGKQREFVANIAARGSTDYANSVKGRFTISRDNAKNTVYLQMNSLKSDDTAVYYCNALVWSGSSYNNWGQGTQVTVSS

SEQ ID NO: 49 - protein, 118 aa, synthetic construct
QVQLVESGGGLVKPGGSLRLSCAASGFSASDVVMAWYRQAPGKQREFVANIAARGSTDYADSVKGRFTISRDNAKNSLYLQMNSLRAEDTAVYYCNALVWNGSSYNNWGQGTQVTVSS

SEQ ID NO: 50 - protein, 118 aa, synthetic construct
QVQLVESGGGLVKPGGSLRLSCAASGFSASDVVMAWYRQAPGKQREFVANIAARGSTDYADSVKGRFTISRDNAKNSLYLQMNSLRAEDTAVYYCNALVWQGSSYNNWGQGTQVTVSS

SEQ ID NO: 51 - protein, 118 aa, synthetic construct
QVQLVESGGGLVKPGGSLRLSCAASGFSASDVVMAWYRQAPGKQREFVANIAARGSTDYADSVKGRFTISRDNAKNSLYLQMNSLRAEDTAVYYCNALVWTGSSYNNWGQGTQVTVSS

SEQ ID NO: 52 - protein, 118 aa, synthetic construct
QVQLVESGGGLVKPGGSLRLSCAASGFSASDVVMAWYRQAPGKQREFVANIAARGSTDYADSVKGRFTISRDNAKNSLYLQMNSLRAEDTAVYYCNALVWSGSSYNNWGQGTQVTVSS

SEQ ID NO: 53 - protein, 118 aa, synthetic construct
QVQLVESGGGLVKPGGSLRLSCAASGFSASDVVMSWYRQAPGKQREFVANIAARGSTDYADSVKGRFTISRDNAKNSLYLQMNSLRAEDTAVYYCNALVWNGSSYNNWGQGTQVTVSS

SEQ ID NO: 54 - protein, 118 aa, synthetic construct
QVQLVESGGGLVKPGGSLRLSCAASGFSASDVVMSWYRQAPGKQREFVAYIAARGSTDYADSVKGRFTISRDNAKNSLYLQMNSLRAEDTAVYYCNALVWNGSSYNNWGQGTQVTVSS

SEQ ID NO: 55 - protein, 118 aa, synthetic construct
QVQLVESGGGLVKPGGSLRLSCAASGFSASDVVMSWYRQAPGKQREFVSYIAARGSTDYADSVKGRFTISRDNAKNSLYLQMNSLRAEDTAVYYCNALVWNGSSYNNWGQGTQVTVSS

SEQ ID NO: 56 - protein, 118 aa, synthetic construct
QVQLVESGGGLVKPGGSLRLSCAASGFSASDVVMSWYRQAPGKQREFVSYIAARGSTDYANSVKGRFTISRDNAKNSLYLQMNSLRAEDTAVYYCNALVWNGSSYNNWGQGTQVTVSS

SEQ ID NO: 57 - protein, 118 aa, synthetic construct
QVQLVESGGGLVKPGGSLRLSCAASGFSASDVVMSWYRQAPGKQREFVSYIAARGSTYYADSVKGRFTISRDNAKNSLYLQMNSLRAEDTAVYYCNALVWNGSSYNNWGQGTQVTVSS

SEQ ID NO: 58 - protein, 118 aa, synthetic construct
QVQLVESGGGLVKPGGSLRLSCAASGFSASDVVMAWYRQAPGKQREFVSNIAARGSTDYADSVKGRFTISRDNAKNSLYLQMNSLRAEDTAVYYCNALVWNGSSYNNWGQGTQVTVSS

SEQ ID NO: 59 - protein, 118 aa, synthetic construct
QVQLVESGGGLVKPGGSLRLSCAASGFSASDVVMAWYRQAPGKQREFVSYIAARGSTDYADSVKGRFTISRDNAKNSLYLQMNSLRAEDTAVYYCNALVWNGSSYNNWGQGTQVTVSS

SEQ ID NO: 60 - protein, 118 aa, synthetic construct
QVQLVESGGGLVKPGGSLRLSCAASGFSASDVVMAWYRQAPGKQREFVSNIAARGSTDYADSVKGRFTISRDNAKNSLYLQMNSLRAEDTAVYYCNALVWTGSSYNNWGQGTQVTVSS

SEQ ID NO: 61 - protein, 118 aa, synthetic construct
QVQLVESGGGLVKPGGSLRLSCAASGFSASDVVMAWYRQAPGKQREFVSNIAARGSTYYADSVKGRFTISRDNAKNSLYLQMNSLRAEDTAVYYCNALVWTGSSYNNWGQGTQVTVSS

SEQ ID NO: 62 - protein, 118 aa, synthetic construct
EVQLVESGGGLVQPGGSLRLSCAASGFSASDVVMSWVRQAPGKQREFVSAIAASSTTYYADSVKGRFTISRDNSKNTLYLQMNSLRAEDTAVYYCNALVWTGSSYNNWGQGTQVTVSS

SEQ ID NO: 63 - protein, 118 aa, synthetic construct
EVQLVESGGGLVQPGGSLRLSCAASGFSASDVVMSWVRQAPGKQREFVAAIAASSTTYYADSVKGRFTISRDNSKNTLYLQMNSLRAEDTAVYYCNALVWTGSSYNNWGQGTQVTVSS

SEQ ID NO: 64 - protein, 118 aa, synthetic construct
QVQLVESGGGLVKPGGSLRLSCAASGFSASDVVMSWIRQAPGKQREFVSYIAASSTTYYADSVKGRFTISRDNAKNSLYLQMNSLRAEDTAVYYCNALVWTGSSYNNWGQGTQVTVSS

SEQ ID NO: 65 - protein, 118 aa, synthetic construct
QVQLVESGGGLVQPGGSLRLSCAASGFSASDVVMSWIRQAPGKQREFVSAIAASSTTYYADSVKGRFTISRDNAKNSLYLQMNSLRAEDTAVYYCNALVWTGSSYNNWGQGTQVTVSS

SEQ ID NO: 66 - protein, 118 aa, synthetic construct
QVQLVESGGGLVKPGGSLRLSCAASGFSASDVVMSWYRQAPGKQREFVAYIAASSTTRYADSVKGRFTISRDNAKNSLYLQMNSLRAEDTAVYYCNALVWTGSSYNNWGQGTQVTVSS

SEQ ID NO: 67 - protein, 118 aa, synthetic construct
QVQLVESGGGLVKPGGSLRLSCAASGFSASDVVMSWYRQAPGKQREFVSYIAASSTTRYADSVKGRFTISRDNAKNSLYLQMNSLRAEDTAVYYCNALVWTGSSYNNWGQGTQVTVSS

SEQ ID NO: 68 - protein, 118 aa, synthetic construct
QVQLVESGGGLVKPGGSLRLSCAASGFSASDVVMAWYRQAPGKQREFVANIAASSTTRYADSVKGRFTISRDNAKNSLYLQMNSLRAEDTAVYYCNALVWTGSSYNNWGQGTQVTVSS

SEQ ID NO: 69 - protein, 118 aa, synthetic construct
QVQLVESGGGLVKPGGSLRLSCAASGFSASDVVMAWYRQAPGKQREFVANIAASSTTYYADSVKGRFTISRDNAKNSLYLQMNSLRAEDTAVYYCNALVWTGSSYNNWGQGTQVTVSS

SEQ ID NO: 70 - protein, 118 aa, synthetic construct
QVQLVESGGGLVKPGGSLRLSCAASGFSASDVVMAWYRQAPGKQREFVSNIAASSTTRYADSVKGRFTISRDNAKNSLYLQMNSLRAEDTAVYYCNALVWTGSSYNNWGQGTQVTVSS

SEQ ID NO: 71 - protein, 118 aa, synthetic construct
QVQLVESGGGLVKPGGSLRLSCAASGFSASDVVMAWYRQAPGKQREFVSNIAASSTTYYADSVKGRFTISRDNAKNSLYLQMNSLRAEDTAVYYCNALVWTGSSYNNWGQGTQVTVSS

SEQ ID NO: 72 - protein, 118 aa, synthetic construct
QVQLVESGGGLVKPGGSLRLSCAASGFSASDVVMAWYRQAPGKQREFVSYIAASSTTYYADSVKGRFTISRDNAKNSLYLQMNSLRAEDTAVYYCNALVWTGSSYNNWGQGTQVTVSS

SEQ ID NO: 73 - protein, 118 aa, synthetic construct
QVQLVESGGGLVKPGGSLRLSCAASGFSASDVVMAWYRQAPGKQREFVSYIAASSTTRYADSVKGRFTISRDNAKNSLYLQMNSLRAEDTAVYYCNALVWTGSSYNNWGQGTQVTVSS

SEQ ID NO: 74 - protein, 118 aa, synthetic construct
QVQLVESGGGLVKPGGSLRLSCAASGFSASDVVMAWYRQAPGKQREFVANLGASSTTRYADSVKGRFTISRDNAKNSLYLQMNSLRAEDTAVYYCNALVWTGSSYNNWGQGTQVTVSS

SEQ ID NO: 75 - protein, 118 aa, synthetic construct
QVQLVESGGGLVKPGGSLRLSCAASGFSASDVVMAWYRQAPGKQREFVSNLGASSTTRYADSVKGRFTISRDNAKNSLYLQMNSLRAEDTAVYYCNALVWTGSSYNNWGQGTQVTVSS

SEQ ID NO: 76 - protein, 118 aa, synthetic construct
QVQLVESGGGLVKPGGSLRLSCAASGFSASDVVMAWYRQAPGKQREFVSNLGASSTTYYADSVKGRFTISRDNAKNSLYLQMNSLRAEDTAVYYCNALVWTGSSYNNWGQGTQVTVSS

SEQ ID NO: 77 - protein, 118 aa, synthetic construct
QVQLVESGGGLVKPGGSLRLSCAASGFSASDVVMAWYRQAPGKQREFVSNLGASSTTNYADSVKGRFTISRDNAKNSLYLQMNSLRAEDTAVYYCNALVWTGSSYNNWGQGTQVTVSS

SEQ ID NO: 78 - protein, 118 aa, synthetic construct
EVQLVESGGGLVKPGGSLRLSCAASGFSAGDVVMAWYRQAPGKQREFVANIAASSTTRYADSVKGRFTISRDNAKNSLYLQMNSLRAEDTAVYYCNALVWTGSSYNNWGQGTQVTVSS

SEQ ID NO: 79 - protein, 118 aa, synthetic construct
EVQLVESGGGLVKPGGSLRLSCAASGFSAGDVVMAWYRQAPGKQREFVSNIAASSTTRYADSVKGRFTISRDNAKNSLYLQMNSLRAEDTAVYYCNALVWTGSSYNNWGQGTQVTVSS

SEQ ID NO: 80 - protein, 118 aa, synthetic construct
EVQLVESGGGLVKPGGSLRLSCAASGFSAGDVVMAWYRQAPGKQREFVSNIAASSTTYYADSVKGRFTISRDNAKNSLYLQMNSLRAEDTAVYYCNALVWTGSSYNNWGQGTQVTVSS

SEQ ID NO: 81 - protein, 118 aa, synthetic construct
QVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSS

SEQ ID NO: 82 - protein, 126 aa, synthetic construct
QVQLVESGGGVVQPGRSLRLSCAASGRSFSRYAMAWVRQAPGKERESVAVIGSGGGRTYYADSVKGRFTISRDNSKNTLYLQMNSLRAEDTAVYYCAARIYRVVGSGTYPAGYDYWGQGTLVTVSS

SEQ ID NO: 83 - protein, 121 aa, synthetic construct
QVQLVESGGGVVQPGRSLRLSCAASGLSFSQYEMHWVRQAPGKEREFVAAISSSGISTYYADSVKGRFTISRDNSKNTLYLQMNSLRAEDTAVYYCAAELVDYHRWGYDYWGQGTLVTVSS

SEQ ID NO: 84 - protein, 124 aa, synthetic construct
QVQLVESGGGVVQPGRSLRLSCAASGLSFSYGTMAWVRQAPGKELELVAALSAGGTITYYADSVKGRFTISRDNSKNTLYLQMNSLRAEDTAVYYCAALGGYSGGSKYPYYDYWGQGTLVTVSS

SEQ ID NO: 85 - protein, 483 aa, synthetic construct
EVQLVESGGGLVKPGGSLRLSCAASGFSAGDVVMAWYRQAPGKQREFVSNIAASSTTRYADSVKGRFTISRDNAKNSLYLQMNSLRAEDTAVYYCNALVWTGSSYNNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSS

SEQ ID NO: 86 - protein, 480 aa, synthetic construct
EVQLVESGGGLVKPGGSLRLSCAASGFSAGDVVMAWYRQAPGKQREFVSNIAASSTTRYADSVKGRFTISRDNAKNSLYLQMNSLRAEDTAVYYCNALVWTGSSYNNWGQGTQVTVSSGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 480 aa, synthetic construct
QVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSSGSGGSGGSGGSGEVQLVESGGGLVKPGGSLRLSCAASGFSAGDVVMAWYRQAPGKQREFVSNIAASSTTRYADSVKGRFTISRDNAKNSLYLQMNSLRAEDTAVYYCNALVWTGSSYNNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 483 aa, synthetic construct
QVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGEVQLVESGGGLVKPGGSLRLSCAASGFSAGDVVMAWYRQAPGKQREFVSNIAASSTTRYADSVKGRFTISRDNAKNSLYLQMNSLRAEDTAVYYCNALVWTGSSYNNWGQGTQVTVSS

SEQ ID NO: 89 - protein, 483 aa, synthetic construct
QVQLVESGGGLVKPGGSLRLSCAASGFSASDVVMAWYRQAPGKQREFVSNIAARGSTDYADSVKGRFTISRDNAKNSLYLQMNSLRAEDTAVYYCNALVWTGSSYNNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSS

SEQ ID NO: 90 - protein, 483 aa, synthetic construct
QVQLVESGGGLVKPGGSLRLSCAASGFSASDVVMAWYRQAPGKQREFVANIAASSTTRYADSVKGRFTISRDNAKNSLYLQMNSLRAEDTAVYYCNALVWTGSSYNNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSS

SEQ ID NO: 91 - protein, 483 aa, synthetic construct
QVQLVESGGGLVKPGGSLRLSCAASGFSASDVVMAWYRQAPGKQREFVANLGASSTTRYADSVKGRFTISRDNAKNSLYLQMNSLRAEDTAVYYCNALVWTGSSYNNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSS

SEQ ID NO: 92 - protein, 483 aa, synthetic construct
EVQLVESGGGLVKPGGSLRLSCAASGFSAGDVVMAWYRQAPGKQREFVANIAASSTTRYADSVKGRFTISRDNAKNSLYLQMNSLRAEDTAVYYCNALVWTGSSYNNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSS

SEQ ID NO: 93 - protein, 483 aa, synthetic construct; identical to SEQ ID NO: 85

SEQ ID NO: 94 - protein, 476 aa, synthetic construct
QVQLVESGGGVVQPGGSLRLSCAASGIDFSDTHMHWVRQAPGKQRELVAIITSGGFTDYVDSVKGRFTISRDNSKNTLYLQMNSLRAEDTAVYYCNARTSTIYWGQGTQVTVSSGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 479 aa, synthetic construct
QVQLVESGGGVVQPGGSLRLSCAASGIDFSDTHMHWVRQAPGKQRELVAIITSGGFTDYVDSVKGRFTISRDNSKNTLYLQMNSLRAEDTAVYYCNARTSTIYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGFTFGPYAMHWVRQAPGKERELVAAIWPSGSITYYADSVKGRFTISRDNSKNTLYLQMNSLRAEDTAVYYCAFGSPTGSITYWGQGTLVTVSS

SEQ ID NO: 96 - protein, 8 aa, synthetic construct
GIFFSDTH

SEQ ID NO: 97 - protein, 7 aa, synthetic construct
ITSGGST

SEQ ID NO: 98 - protein, 8 aa, synthetic construct
NARRTTIY

SEQ ID NO: 99 - protein, 8 aa, synthetic construct
GIDFSDTH

SEQ ID NO: 100 - protein, 7 aa, synthetic construct
ITSGGFT

SEQ ID NO: 101 - protein, 8 aa, synthetic construct
NARRTTIY

SEQ ID NO: 102 - protein, 8 aa, synthetic construct
GFDFSDTH

SEQ ID NO: 103 - protein, 7 aa, synthetic construct
ITSGAFT

SEQ ID NO: 104 - protein, 8 aa, synthetic construct
NARRTTIY

SEQ ID NO: 105 - protein, 8 aa, synthetic construct
GIDFSDTH

SEQ ID NO: 106 - protein, 7 aa, synthetic construct
ITSGGFT

SEQ ID NO: 107 - protein, 8 aa, synthetic construct
NARKTTIY

SEQ ID NO: 108 - protein, 8 aa, synthetic construct
GIFFSGVH

SEQ ID NO: 109 - protein, 7 aa, synthetic construct
ITDSGSA

SEQ ID NO: 110 - protein, 8 aa, synthetic construct
NARTTTIY

SEQ ID NO: 111 - protein, 8 aa, synthetic construct
GIDFSDTH

SEQ ID NO: 112 - protein, 7 aa, synthetic construct
ITSGGFT

SEQ ID NO: 113 - protein, 8 aa, synthetic construct
NARRSTIY

SEQ ID NO: 114 - protein, 8 aa, synthetic construct
GFSASDVV

SEQ ID NO: 115 - protein, 7 aa, synthetic construct
IAARGST

SEQ ID NO: 116 - protein, 12 aa, synthetic construct
NALVWNGSSYNN

SEQ ID NO: 117 - protein, 8 aa, synthetic construct
GFSASDVV

SEQ ID NO: 118 - protein, 7 aa, synthetic construct
IAASSTT

SEQ ID NO: 119 - protein, 12 aa, synthetic construct
NALVWNGSSYNN

SEQ ID NO: 120 - protein, 8 aa, synthetic construct
GFSASDVV

SEQ ID NO: 121 - protein, 7 aa, synthetic construct
LGASSTT

SEQ ID NO: 122 - protein, 12 aa, synthetic construct
NALVWNGSSYNN

SEQ ID NO: 123 - protein, 8 aa, synthetic construct
GFSAGDVV

SEQ ID NO: 124 - protein, 7 aa, synthetic construct
IAASSTT

SEQ ID NO: 125 - protein, 12 aa, synthetic construct
NALVWNGSSYNN

SEQ ID NO: 126 - protein, 8 aa, synthetic construct
GIDFSDTH

SEQ ID NO: 127 - protein, 7 aa, synthetic construct
ITSGGST

SEQ ID NO: 128 - protein, 8 aa, synthetic construct
NARRSTIY

SEQ ID NO: 129 - protein, 8 aa, synthetic construct
GFDFSDTH

SEQ ID NO: 130 - protein, 7 aa, synthetic construct
ITSGGST

SEQ ID NO: 131 - protein, 8 aa, synthetic construct
NARRTTIY

SEQ ID NO: 132 - protein, 8 aa, synthetic construct
GIDFSDTH

SEQ ID NO: 133 - protein, 7 aa, synthetic construct
ITSGGFT

SEQ ID NO: 134 - protein, 8 aa, synthetic construct
NARTSTIY

SEQ ID NO: 135 - protein, 8 aa, synthetic construct
GIDFSDTH

SEQ ID NO: 136 - protein, 7 aa, synthetic construct
ITSSGST

SEQ ID NO: 137 - protein, 8 aa, synthetic construct
NARRSTIL

SEQ ID NO: 138 - protein, 8 aa, synthetic construct
GIDFDDTH

SEQ ID NO: 139 - protein, 7 aa, synthetic construct
ITSGGLT

SEQ ID NO: 140 - protein, 8 aa, synthetic construct
NARTSTIY

SEQ ID NO: 141 - protein, 8 aa, synthetic construct
GIDFNDTH

SEQ ID NO: 142 - protein, 7 aa, synthetic construct
ITSRGST

SEQ ID NO: 143 - protein, 8 aa, synthetic construct
NARRSTLY

SEQ ID NO: 144 - protein, 8 aa, synthetic construct
GIDFSDLH

SEQ ID NO: 145 - protein, 7 aa, synthetic construct
ITSGGST

SEQ ID NO: 146 - protein, 8 aa, synthetic construct
NARSSTIY

SEQ ID NO: 147 - protein, 8 aa, synthetic construct
GFDFSDTH

SEQ ID NO: 148 - protein, 7 aa, synthetic construct
ITGGGST

SEQ ID NO: 149 - protein, 8 aa, synthetic construct
LARRTTIY

SEQ ID NO: 150 - protein, 8 aa, synthetic construct
GFDFSDTH

SEQ ID NO: 151 - protein, 7 aa, synthetic construct
ITSGGST

SEQ ID NO: 152 - protein, 8 aa, synthetic construct
NARLTTIY

SEQ ID NO: 153 - protein, 8 aa, synthetic construct
GFDFSDTF

SEQ ID NO: 154 - protein, 7 aa, synthetic construct
ITSGGST

SEQ ID NO: 155 - protein, 8 aa, synthetic construct
NARRTTIY

SEQ ID NO: 156 - protein, 8 aa, synthetic construct
GFDFPDTH

SEQ ID NO: 157 - protein, 7 aa, synthetic construct
ITSGGFT

SEQ ID NO: 158 - protein, 8 aa, synthetic construct
RARRTTIY

SEQ ID NO: 159 - protein, 8 aa, synthetic construct
GFDFSDTA

SEQ ID NO: 160 - protein, 7 aa, synthetic construct
ITSGGSV

SEQ ID NO: 161 - protein, 8 aa, synthetic construct
RARRTTIY

SEQ ID NO: 162 - protein, 8 aa, synthetic construct
GFDFSMTH

SEQ ID NO: 163 - protein, 7 aa, synthetic construct
ITSGGKT

SEQ ID NO: 164 - protein, 8 aa, synthetic construct
NARRATIY

SEQ ID NO: 165 - protein, 8 aa, synthetic construct
GFDFEDTH

SEQ ID NO: 166 - protein, 7 aa, synthetic construct
ITSGCST

SEQ ID NO: 167 - protein, 8 aa, synthetic construct
NARRATIY

SEQ ID NO: 168 - protein, 8 aa, synthetic construct
GFSASDVV

SEQ ID NO: 169 - protein, 7 aa, synthetic construct
IAARGST

SEQ ID NO: 170 - protein, 12 aa, synthetic construct
NALVWQGSSYNN

SEQ ID NO: 171 - protein, 8 aa, synthetic construct
GFSASDVV

SEQ ID NO: 172 - protein, 7 aa, synthetic construct
IAARGST

SEQ ID NO: 173 - protein, 12 aa, synthetic construct
NALVWTGSSYNN

SEQ ID NO: 174 - protein, 8 aa, synthetic construct
GFSASDVV

SEQ ID NO: 175 - protein, 7 aa, synthetic construct
IAARGST

SEQ ID NO: 176 - protein, 12 aa, synthetic construct
NALVWSGSSYNN

SEQ ID NO: 177 - protein, 8 aa, synthetic construct
GFSASDVV

SEQ ID NO: 178 - protein, 7 aa, synthetic construct
IAASSTT

SEQ ID NO: 179 - protein, 12 aa, synthetic construct
NALVWTGSSYNN

SEQ ID NO: 180 - protein, 8 aa, synthetic construct
GFSASDVV

SEQ ID NO: 181 - protein, 7 aa, synthetic construct
LGASSTT

SEQ ID NO: 182 - protein, 12 aa, synthetic construct
NALVWTGSSYNN

SEQ ID NO: 183 - protein, 8 aa, synthetic construct
GFSAGDVV

SEQ ID NO: 184 - protein, 7 aa, synthetic construct
IAASSTT

SEQ ID NO: 185 - protein, 12 aa, synthetic construct
NALVWTGSSYNN

SEQ ID NO: 186 - protein, 8 aa, synthetic construct
GFTFGPYA

SEQ ID NO: 187 - protein, 8 aa, synthetic construct
IWPSGSIT

SEQ ID NO: 188 - protein, 11 aa, synthetic construct
AFGSPTGSITY

SEQ ID NO: 189 - protein, 8 aa, synthetic construct
GRSFSRYA

SEQ ID NO: 190 - protein, 8 aa, synthetic construct
IGSGGGRT

SEQ ID NO: 191 - protein, 19 aa, synthetic construct
AARIYRVVGSGTYPAGYDY

SEQ ID NO: 192 - protein, 8 aa, synthetic construct
GLSFSQYE

SEQ ID NO: 193 - protein, 8 aa, synthetic construct
ISSSGIST

SEQ ID NO: 194 - protein, 14 aa, synthetic construct
AAELVDYHRWGYDY

SEQ ID NO: 195 - protein, 8 aa, synthetic construct
GLSFSYGT

SEQ ID NO: 196 - protein, 8 aa, synthetic construct
LSAGGTIT

SEQ ID NO: 197 - protein, 17 aa, synthetic construct
AALGGYSGGSKYPYYDY

SEQ ID NO: 198 - protein, 8 aa, synthetic construct
  VARIANT 4: note = X can be K, T or R
NARXTTIY

SEQ ID NO: 199 - protein, 12 aa, synthetic construct
  VARIANT 6: note = X can be Q, T, S, or N
NALVWXGSSYNN

SEQ ID NO: 200 - protein, 118 aa, synthetic construct
QVQLVESGGGLVQAGGSLRLSCAASGFSASDVVMAWYRQAPGKQREFVANLGASSTTRYADSVKGRFTISRDDAKNTVSLQMNSLKSEDTAVYYCNALVWTGSSYNNWGQGTQVTVSS

SEQ ID NO: 201 - protein, 118 aa, synthetic construct
EVQLVESGGGSVQDGGSLTLSCAASGFSAGDVVMAWYRQAPGKQREFVANIAASSTTRYTDSVKGRFTISRDNAKNTVYLQMNSLKSEDTAVYYCNALVWTGSSYNNWGQGTQVTVSS

SEQ ID NO: 202 - protein, 12 aa, synthetic construct
GSGGSGGSGGSG

SEQ ID NO: 203 - protein, 15 aa, synthetic construct
GSGGSGGSGGSGGSG

SEQ ID NO: 204 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 5 aa, synthetic construct
GGGGS

(19) United States
(12) Patent Application Publication
(10) Pub. No.: US 2026/0365734 A1
(43) Pub. Date: Nov. 19, 2026

(54) MULTISPECIFIC ANTIBODIES AND USES THEREOF

(71) Applicant: BRIGHT BIOLOGICS LLC, Lewes, DE (US)

(72) Inventors: Ming Yang, Mountain View, CA (US); Hongyan Zhong, Mountain View, CA (US); Hsieh Li, Mountain View, CA (US); Daniel Guo, San Jose, CA (US)

(21) Appl. No.: 19/099,275

(22) PCT Filed: Aug. 25, 2023
(86) PCT No.: PCT/US2023/072884

§ 371 Date: Jan. 28, 2025

Related U.S. Application Data
(60) Provisional application No. 63/401,086, filed on Aug. 25, 2022.

(51) Int. Cl. C07K 16/28; A61K 39/00; A61P 35/00; C07K 16/22

(52) U.S. Cl. CPC C07K 16/2827; A61P 35/00; C07K 16/22; A61K 2039/505; C07K 2317/24; C07K 2317/31; C07K 2317/35; C07K 2317/52; C07K 2317/53; C07K 2317/565; C07K 2317/569; C07K 2317/76; C07K 2317/92

(57) ABSTRACT

This disclosure relates to multi specific antibodies (e.g., bispecific antibodies) or antigen-binding fragments thereof. In one aspect, the multispecific antibodies or antigen-binding fragments thereof binds to PD-LI and/or VEGF, or a combination thereof. More specifically, the antibodies or antigen-bindng fragments comprise sequence-specified heavy and light chain variable regions, further comprising sequence-specified CDRs 1, 2, and 3, respectively.

### CROSS-REFERENCE TO RELATED APPLICATION

This disclosure claims priority to and benefit of U.S. Provisional Patent Application Ser. No. 63/401,086, filed Aug. 25, 2022, which is incorporated herein by reference in its entirety.

### SEQUENCE LISTING

This application contains a Sequence Listing that has been submitted electronically as an XML file named 52501-0007WO1.xml. The XML file, created on Aug. 22, 2023, is 194,051 bytes in size. The material in the XML file is hereby incorporated by reference in its entirety.

### TECHNICAL FIELD

This disclosure relates to multispecific antibodies or antigen-binding fragments thereof.

### BACKGROUND

A multispecific antibody is an artificial protein that can simultaneously bind to two or more different epitopes. This opens up a wide range of applications, including redirecting T cells to tumor cells, blocking two different signaling pathways simultaneously, dual targeting of different disease mediators, and delivering payloads to targeted sites. The approval of catumaxomab (anti-EpCAM and anti-CD3) and blinatumomab (anti-CD19 and anti-CD3) has become a major milestone in the development of multispecific antibodies.

As multispecific antibodies have various applications, there is a need to continue to develop various therapeutics based on multispecific antibodies.

### SUMMARY

This disclosure relates to antibodies or antigen-binding fragments, wherein the antibodies or antigen-binding fragments specifically bind to PD-L1 and/or VEGF, or a combination thereof. In some embodiments, the disclosure relates to development of PD-L1/VEGF targeting bispecific antibodies.

In one aspect, the disclosure is related to an antibody or antigen-binding fragment thereof that binds to PD-L1 (Programmed death-ligand 1), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 96, 97, and 98, respectively;
(2) the selected VHH CDRs 1, 2, 3 amino acid sequences are set forth in SEQ ID NOs: 99, 100, and 101, respectively;
(3) the selected VHH CDRs 1, 2, 3 amino acid sequences are set forth in SEQ ID NOs: 102, 103 and 104, respectively;
(4) the selected VHH CDRs 1, 2, 3 amino acid sequences are set forth in SEQ ID NOs: 105, 106, and 107, respectively;
(5) the selected VHH CDRs 1, 2, 3 amino acid sequences are set forth in SEQ ID NOs: 108, 109, and 110, respectively;
(6) the selected VHH CDRs 1, 2, 3 amino acid sequences are set forth in SEQ ID NOs: 111, 112, and 113, respectively;
(7) the selected VHH CDRs 1, 2, 3 amino acid sequences are set forth in SEQ ID NOs: 114, 115, and 116, respectively;
(8) the selected VHH CDRs 1, 2, 3 amino acid sequences are set forth in SEQ ID NOs: 117, 118, and 119, respectively;
(9) the selected VHH CDRs 1, 2, 3 amino acid sequences are set forth in SEQ ID NOs: 120, 121, and 122, respectively;
(10) the selected VHH CDRs 1, 2, 3 amino acid sequences are set forth in SEQ ID NOs: 123, 124, and 125, respectively;
(11) the selected VHH CDRs 1, 2, 3 amino acid sequences are set forth in SEQ ID NOs: 126, 127, and 128, respectively;
(12) the selected VHH CDRs 1, 2, 3 amino acid sequences are set forth in SEQ ID NOs: 129, 130, and 131, respectively;
(13) the selected VHH CDRs 1, 2, 3 amino acid sequences are set forth in SEQ ID NOs: 132, 133, and 134, respectively;
(14) the selected VHH CDRs 1, 2, 3 amino acid sequences are set forth in SEQ ID NOs: 135, 136, and 137, respectively;
(15) the selected VHH CDRs 1, 2, 3 amino acid sequences are set forth in SEQ ID NOs: 138, 139, and 140, respectively;
(16) the selected VHH CDRs 1, 2, 3 amino acid sequences are set forth in SEQ ID NOs: 141, 142, and 143, respectively;
(17) the selected VHH CDRs 1, 2, 3 amino acid sequences are set forth in SEQ ID NOs: 144, 145, and 146, respectively;
(18) the selected VHH CDRs 1, 2, 3 amino acid sequences are set forth in SEQ ID NOs:

147, 148, and 149, respectively;
(19) the selected VHH CDRs 1, 2, 3 amino acid sequences are set forth in SEQ ID NOs: 150, 151, and 152, respectively;
(20) the selected VHH CDRs 1, 2, 3 amino acid sequences are set forth in SEQ ID NOs: 153, 154, and 155, respectively;
(21) the selected VHH CDRs 1, 2, 3 amino acid sequences are set forth in SEQ ID NOs: 156, 157, and 158, respectively;
(22) the selected VHH CDRs 1, 2, 3 amino acid sequences are set forth in SEQ ID NOs: 159, 160, and 161, respectively;
(23) the selected VHH CDRs 1, 2, 3 amino acid sequences are set forth in SEQ ID NOs:

162, 163, and 164, respectively;
(24) the selected VHH CDRs 1, 2, 3 amino acid sequences are set forth in SEQ ID NOs: 165, 166, and 167, respectively;
(25) the selected VHH CDRs 1, 2, 3 amino acid sequences are set forth in SEQ ID NOs: 168, 169, and 170, respectively;
(26) the selected VHH CDRs 1, 2, 3 amino acid sequences are set forth in SEQ ID NOs: 171, 172, and 173, respectively;
(27) the selected VHH CDRs 1, 2, 3 amino acid sequences are set forth in SEQ ID NOs: 174, 175, and 176, respectively;
(28) the selected VHH CDRs 1, 2, 3 amino acid sequences are set forth in SEQ ID NOs: 177, 178, and 179, respectively;
(29) the selected VHH CDRs 1, 2, 3 amino acid sequences are set forth in SEQ ID NOs: 180, 181, and 182, respectively; and
(30) the selected VHH CDRs 1, 2, 3 amino acid sequences are set forth in SEQ ID NOs: 183, 184, and 185, respectively.

In some embodiments, the VHH comprises CDRs 1, 2, 3 with the amino acid sequences set forth in SEQ ID NOs: 183, 184, and 185, respectively.

In some embodiments, the VHH comprises CDRs 1, 2, 3 with the amino acid sequences set forth in SEQ ID NOs: 171, 172, and 173, respectively.

In some embodiments, the VHH comprises CDRs 1, 2, 3 with the amino acid sequences set forth in SEQ ID NOs: 177, 178, and 179, respectively.

In some embodiments, the VHH comprises CDRs 1, 2, 3 with the amino acid sequences set forth in SEQ ID NOs: 180, 181, and 182, respectively.

In some embodiments, the VHH comprises CDRs 1, 2, 3 with the amino acid sequences set forth in SEQ ID NOs: 132, 133, and 134, respectively.

In one aspect, the disclosure is related to an antibody or antigen-binding fragment thereof that binds to PD-L1 comprising a heavy-chain antibody variable domain (VHH) comprising an amino acid sequence that is at least 80% identical to a selected VHH sequence, wherein the selected VHH sequence is selected from the group consisting of SEQ ID NOs: 1-80.

In some embodiments, the VHH comprises the sequence of SEQ ID NO: 79.

In some embodiments, the VHH comprises the sequence of SEQ ID NO: 60.

In some embodiments, the VHH comprises the sequence of SEQ ID NO: 68.

In some embodiments, the VHH comprises the sequence of SEQ ID NO: 74.

In some embodiments, the VHH comprises the sequence of SEQ ID NO: 78.

In some embodiments, wherein the VHH comprises the sequence of SEQ ID NO: 33.

In some embodiments, the antibody or antigen-binding fragment specifically binds to PD-L1.

In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to VEGF (Vascular endothelial growth factor), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 186, 187, and 188, respectively;
(2) the selected VHH CDRs 1, 2, 3 amino acid sequences are set forth in SEQ ID NOs: 189, 190, and 191, respectively;
(3) the selected VHH CDRs 1, 2, 3 amino acid sequences are set forth in SEQ ID NOs: 192, 193, and 194, respectively; and
(4) the selected VHH CDRs 1, 2, 3 amino acid sequences are set forth in SEQ ID NOs: 195, 196, and 197, respectively.

In some embodiments, the VHH comprises CDRs 1, 2, 3 with the amino acid sequences set forth in SEQ ID NOs: 186, 187, and 188, respectively.

In some embodiments, the VHH comprises CDRs 1, 2, 3 with the amino acid sequences set forth in SEQ ID NOs: 189, 190, and 191, respectively.

In some embodiments, the VHH comprises CDRs 1, 2, 3 with the amino acid sequences set forth in SEQ ID NOs: 192, 193, and 194, respectively.

In some embodiments, the VHH comprises CDRs 1, 2, 3 with the amino acid sequences set forth in SEQ ID NOs: 195, 196, and 197, respectively.

In one aspect, the disclosure is related to an antibody or antigen-binding fragment thereof that binds to VEGF comprising a heavy-chain antibody variable domain (VHH) comprising an amino acid sequence that is at least 80% identical to a selected VHH sequence, wherein the selected VHH sequence is selected from the group consisting of SEQ ID NOs: 81-84.

In some embodiments, the VHH comprises the sequence of SEQ ID NO: 81.

In some embodiments, the VHH comprises the sequence of SEQ ID NO: 82.

In some embodiments, the VHH comprises the sequence of SEQ ID NO: 83.

In some embodiments, the VHH comprises the sequence of SEQ ID NO: 84.

In some embodiments, the antibody or antigen-binding fragment specifically binds to VEGF.

In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof described herein.

In one aspect, the disclosure is related to a multi-specific antibody or antigen-binding fragment thereof, comprising a first VHH (VHH1) that specifically binds to VEGF, a second VHH (VHH2) that specifically binds to PD-L1.

In some embodiments, the multi-specific antibody or antigen-binding fragment thereof further comprises a third VHH (VHH3) that specifically binds to VEGF, and a fourth VHH (VHH4) that specifically binds to PD-L1.

In some embodiments, the VHH1 and/or the VHH3 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 28.

In some embodiments, the VHH1 and/or the VHH3 comprises an amino acid sequence that is at least 80% identical to a selected VHH sequence, wherein the selected VHH sequence is selected from the group consisting of SEQ ID NOs: 1-80.

In some embodiments, the VHH2 and/or the VHH4 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 29.

In some embodiments, the VHH3 and the VHH4 comprise an amino acid sequence that is at least 80% identical to a selected VHH sequence, wherein the selected VHH sequence is selected from the group consisting of SEQ ID NOs: 81-84.

In some embodiments, the multi-specific antibody or antigen-binding fragment thereof comprises a human IgG Fc.

In some embodiments, the VHH1 and VHH3 are linked to the N-terminus or the C-terminus of the human IgG Fc.

In some embodiments, the VHH2 and VHH4 are linked to the N-terminus or the C-terminus of the human IgG Fc.

In one aspect, the disclosure is related to a polypeptide complex, comprising
(a) a first polypeptide comprising from N-terminus to C-terminus: a first heavy-chain antibody variable domain (VHH1), a first hinge region, a first Fc region, and a second VHH (VHH2); and
(b) a second polypeptide comprising from N-terminus to C-terminus: a third VHH (VHH3), a second hinge region, a second Fc region, and a fourth VHH (VHH4), wherein the VHH1 and the VHH3 specifically bind to PD-L1, and the VHH2 and the VHH4 specifically bind to VEGF.

In some embodiments, the first polypeptide comprises a sequence that is at least 80% identical to SEQ ID NO: 85, 89, 90, 91, 92, 93, or 95; and/or wherein the second polypeptide comprises a sequence that is at least 80% identical to SEQ ID NO: 85, 89, 90, 91, 92, 93, or 95.

In some embodiments, the VHH2 is linked to the C-terminus of the first Fc region via a first linker peptide sequence.

In some embodiments, the VHH4 is linked to the C-terminus of the second Fc region via a second linker peptide sequence.

In some embodiments, the first and/or the second linker peptide sequence is at least 80% identical to SEQ ID NO: 202 or 203.

In one aspect, the disclosure is related to a polypeptide complex, comprising
(a) a first polypeptide comprising from N-terminus to C-terminus: a VHH1, a VHH2, a first hinge region, a first Fc region; and
(b) a second polypeptide comprising from N-terminus to C-terminus: a VHH3, a VHH4, a second hinge region, and a second Fc region, wherein the VHH1 and the VHH3 specifically bind to PD-L1 and the VHH2 and the VHH4 specifically bind to VEGF.

In some embodiments, the VHH1 is linked to the N-terminus of the VHH2 via a first linker peptide sequence.

In some embodiments, the VHH3 is linked to the N-terminus of the VHH4 via a second linker peptide sequence.

In some embodiments, the first polypeptide comprises a sequence that is at least 80% identical to SEQ ID NO: 86 or 94; and/or wherein the second polypeptide comprises a sequence that is at least 80% identical to SEQ ID NO: 86 or 94.

In some embodiments, the first and/or the second linker peptide sequence is at least 80% identical to SEQ ID NO: 202 or 203.

In one aspect, the disclosure is related to a polypeptide complex, comprising
(a) a first polypeptide comprising from N-terminus to C-terminus: a VHH2, a VHH1, a first hinge region, a first Fc region; and
(b) a second polypeptide comprising from N-terminus to C-terminus: a VHH4, a VHH3, a second hinge region, and a second Fc region, wherein the VHH1 and the VHH3 specifically bind to PD-L1 and the VHH2 and the VHH4 specifically bind to VEGF.

In some embodiments, the VHH2 is linked to the N-terminus of the VHH1 via a first linker peptide sequence.

In some embodiments, the VHH4 is linked to the N-terminus of the VHH3 via a second linker peptide sequence.

In some embodiments, the first polypeptide comprises a sequence that is at least 80% identical to SEQ ID NO: 87; and/or wherein the second polypeptide comprises a sequence that is at least 80% identical to SEQ ID NO: 87.

In some embodiments, the first and/or the second linker peptide sequence is at least 80% identical to SEQ ID NO: 202 or 203.

In one aspect, the disclosure is related to a polypeptide complex, comprising
(a) a first polypeptide comprising from N-terminus to C-terminus: a VHH2, a first hinge region, a first Fc region, and VHH1; and
(b) a second polypeptide comprising from N-terminus to C-terminus: a VHH4, a second hinge region, a second Fc region, and a VHH3, wherein the VHH1 and the VHH3 specifically bind to PD-L1, and the VHH2 and the VHH4 specifically bind to VEGF.

In some embodiments, the first polypeptide comprises a sequence that is at least 80% identical to SEQ ID NO: 88; and/or wherein the second polypeptide comprises a sequence that is at least 80% identical to SEQ ID NO: 88.

In some embodiments, the VHH1 is linked to the C-terminus of the first Fc region via a first linker peptide sequence.

In some embodiments, the VHH3 is linked to the C-terminus of the second Fc region via a second linker peptide sequence.

In some embodiments, the first and/or the second linker peptide sequence is at least 80% identical to SEQ ID NO: 202 or 203.

In some embodiments, the VHH1 and/or the VHH3 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 28.

In some embodiments, the VHH1 and/or the VHH3 comprises an amino acid sequence that is at least 80% identical to a selected VHH sequence, wherein the selected VHH sequence is selected from the group consisting of SEQ ID NOs: 1-80.

In some embodiments, the VHH2 and/or the VHH4 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 29.

In some embodiments, the VHH2 and/or the VHH4 comprise an amino acid sequence that is at least 80% identical to a selected VHH sequence, wherein the selected VHH sequence is selected from the group consisting of SEQ ID NOs: 81-84.

In one aspect, the disclosure is related to an antibody or antigen-binding fragment thereof that binds to PD-L1 (Programmed death-ligand 1), comprising: a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence, wherein the selected VHH CDRs 1, 2, and 3 amino acid sequences are one of the following:
(1) the selected VHH CDR3 is NARX₁TTIY (SEQ ID NO: 198), wherein X₁=K, T, or R; the selected VHH CDR 1 is selected from the group consisting of SEQ ID NOS: 96, 99, 102, 105, 108, 111, 126 and 129; and the selected VHH CDR2 is selected from the group consisting of SEQ ID NOS: 97, 100, 103, 106, 109, 112, 127 and 130;
(2) the selected VHH CDR3 is NALVWX₂GSSYNN (SEQ ID NO: 199), wherein X²=Q, T, S, or N; the selected VHH CDR1 is selected from the group consisting of SEQ ID NOS: 114, 117, 120, 123, 168, 171, 174, 177, 180, and 183; and the selected VHH CDR2 is selected from the group consisting of SEQ ID NOS: 115, 118, 121, 124, 169, 172, 175, 178, 181, and 184; and
(3) the selected VHH CDR3 is selected from the group consisting of SEQ ID NOS: 134, 137, 140, 143, 146, 149, 152, 155, 158, 161, 164, and 167; the selected VHH CDR 1 is selected from the group consisting of SEQ ID NOS: 132, 135, 138, 141, 144, 147, 150, 153, 156, 159, 162, and 165; and the selected VHH CDR2 is selected from the group consisting of SEQ ID NOS: 133, 136, 139, 142, 145, 148, 151, 154, 157, 160, 163, and 166.

In some embodiments, the selected VHH CDRs 1, 2, and 3 amino acid sequences are one of the following:
(1) the selected VHH CDRs 1, 2, 3 amino acid sequences are set forth in SEQ ID NOs: 96, 97, and 98, respectively;
(2) the selected VHH CDRs 1, 2, 3 amino acid sequences are set forth in SEQ ID NOs: 99, 100, and 101, respectively;
(3) the selected VHH CDRs 1, 2, 3 amino acid sequences are set forth in SEQ ID NOs: 102, 103 and 104, respectively;
(4) the selected VHH CDRs 1, 2, 3 amino acid sequences are set forth in SEQ ID NOs: 105, 106, and 107, respectively;
(5) the selected VHH CDRs 1, 2, 3 amino acid sequences are set forth in SEQ ID NOs: 108, 109, and 110, respectively;
(6) the selected VHH CDRs 1, 2, 3 amino acid sequences are set forth in SEQ ID NOs: 111, 112, and 113, respectively;
(7) the selected VHH CDRs 1, 2, 3 amino acid sequences are set forth in SEQ ID NOs: 114, 115, and 116, respectively;
(8) the selected VHH CDRs 1, 2, 3 amino acid sequences are set forth in SEQ ID NOs: 117, 118, and 119, respectively;
(9) the selected VHH CDRs 1, 2, 3 amino acid sequences are set forth in SEQ ID NOs: 120, 121, and 122, respectively;
(10) the selected VHH CDRs 1, 2, 3 amino acid sequences are set forth in SEQ ID NOs: 123, 124, and 125, respectively;
(11) the selected VHH CDRs 1, 2, 3 amino acid sequences are set forth in SEQ ID NOs: 126, 127, and 128, respectively;
(12) the selected VHH CDRs 1, 2, 3 amino acid sequences are set forth in SEQ ID NOs:

129, 130, and 131, respectively;
(13) the selected VHH CDRs 1, 2, 3 amino acid sequences are set forth in SEQ ID NOs: 132, 133, and 134, respectively;
(14) the selected VHH CDRs 1, 2, 3 amino acid sequences are set forth in SEQ ID NOs: 135, 136, and 137, respectively;
(15) the selected VHH CDRs 1, 2, 3 amino acid sequences are set forth in SEQ ID NOs: 138, 139, and 140, respectively;
(16) the selected VHH CDRs 1, 2, 3 amino acid sequences are set forth in SEQ ID NOs: 141, 142, and 143, respectively;
(17) the selected VHH CDRs 1, 2, 3 amino acid sequences are set forth in SEQ ID NOs: 144, 145, and 146, respectively;
(18) the selected VHH CDRs 1, 2, 3 amino acid sequences are set forth in SEQ ID NOs: 147, 148, and 149, respectively;
(19) the selected VHH CDRs 1, 2, 3 amino acid sequences are set forth in SEQ ID NOs: 150, 151, and 152, respectively;
(20) the selected VHH CDRs 1, 2, 3 amino acid sequences are set forth in SEQ ID NOs: 153, 154, and 155, respectively;
(21) the selected VHH CDRs 1, 2, 3 amino acid sequences are set forth in SEQ ID NOs: 156, 157, and 158, respectively;
(22) the selected VHH CDRs 1, 2, 3 amino acid sequences are set forth in SEQ ID NOs: 159, 160, and 161, respectively;
(23) the selected VHH CDRs 1, 2, 3 amino acid sequences are set forth in SEQ ID NOs: 162, 163, and 164, respectively;
(24) the selected VHH CDRs 1, 2, 3 amino acid sequences are set forth in SEQ ID NOs: 165, 166, and 167, respectively;
(25) the selected VHH CDRs 1, 2, 3 amino acid sequences are set forth in SEQ ID NOs: 168, 169, and 170, respectively;
(26) the selected VHH CDRs 1, 2, 3 amino acid sequences are set forth in SEQ ID NOs: 171, 172, and 173, respectively;
(27) the selected VHH CDRs 1, 2, 3 amino acid sequences are set forth in SEQ ID NOs: 174, 175, and 176, respectively;
(28) the selected VHH CDRs 1, 2, 3 amino acid sequences are set forth in SEQ ID NOs: 177, 178, and 179, respectively;
(29) the selected VHH CDRs 1, 2, 3 amino acid sequences are set forth in SEQ ID NOs: 180, 181, and 182, respectively; and
(30) the selected VHH CDRs 1, 2, 3 amino acid sequences are set forth in SEQ ID NOs: 183, 184, and 185, respectively.

In one aspect, the disclosure is related to a nucleic acid comprising a polynucleotide encoding the antibody or antigen-binding fragment thereof described herein, the multi-specific antibody or antigen-binding fragment thereof described herein, or the polypeptide complex described herein.

In some embodiments, the nucleic acid is a DNA (e.g., cDNA) or RNA (e.g., mRNA).

In one aspect, the disclosure is related to a vector comprising one or more of the nucleic acids described herein.

In one aspect, the disclosure is related to a cell comprising the vector described herein.

In some embodiments, the cell is a CHO cell.

In one aspect, the disclosure is related to a cell comprising one or more of the nucleic acids described herein.

In one aspect, the disclosure is related to a method of producing an antibody or an antigen-binding fragment thereof, the method comprising
(a) culturing the cell described herein under conditions sufficient for the cell to produce the antibody or the antigen-binding fragment; and
(b) collecting the antibody or the antigen-binding fragment produced by the cell.

In one aspect, the disclosure is related to an antibody-drug conjugate comprising the antibody or antigen-binding fragment thereof described herein, the multi-specific antibody or antigen-binding fragment thereof described herein, or the polypeptide complex described herein, covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof described herein, the multi-specific antibody or antigen-binding fragment thereof described herein, or the polypeptide complex described herein, or the antibody-drug conjugate described herein, to the subject.

In some embodiments, the subject has a cancer expressing PD-L1.

In some embodiments, the subject has a cancer expressing VEGF.

In some embodiments, the cancer is colon cancer, rectum cancer, lung cancer, breast cancer, kidney cancer, hepatocellular carcinoma, renal cancer, endometrial carcinoma, pancreatic cancer, head and neck cancer or late-stage solid tumor.

In some embodiments, the cancer is non-small cell lung cancer (NSCLC).

In one aspect, the disclosure is related to a method of decreasing the rate of tumor growth, the method comprising contacting a tumor cell with an effective amount of a composition comprising the antibody or antigen-binding fragment thereof described herein, the multi-specific antibody or antigen-binding fragment thereof described herein, or the polypeptide complex described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, the multi-specific antibody or antigen-binding fragment thereof described herein, or the polypeptide complex described herein, or the antibody-drug conjugate described herein.

In one aspect, the disclosure is related to a pharmaceutical composition comprising the antibody or antigen-binding fragment thereof described herein, the multi-specific antibody or antigen-binding fragment thereof described herein, or the polypeptide complex described herein, or the antibody-drug conjugate described herein, and a pharmaceutically acceptable carrier.

In one aspect, the disclosure provides an antibody or antigen-binding fragment thereof that specifically binds to PD-L1. In one aspect, the disclosure provides an antibody or antigen-binding fragment thereof that specifically binds to VEGF.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)2, and Fv fragments, ScFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to VEGF may be referred to as a VEGF-specific antibody or an anti-VEGF antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of the heavy chain of a heavy chain only antibody, and the variant thereof. A heavy-chain antibody is an antibody which consists only of two heavy chains but can still specifically bind to an antigen. In some embodiments, the VHH is a humanized VHH.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A shows whole cell binding (WCB) of VHH bivalent antibodies to human PD-L1 (h-PD-L1) as determined by flowcytometry assays. X axis is values of antibody concentration in nanomolar. Y axis is values of Median Fluorescence Intensity of AlexaFlour 488. Atezolizumab (Atezo) is a reference anti-h-PD-L1 antibody from Roche. 1F4, 5C12, 6D2, 4H8, 6A10, 1G4, and 6D1 are anti-human PD-L1 VHH-Fc clones.

FIG. 1B shows the potencies of lead bivalent antibodies in blocking human PD-L1 activity as determined using luciferase reporter assays. Human PD-L1 stably transfected CHO cells were cultured with PD1 and NFAT reporter gene stably transfected Jurkat cells in the presence of anti-h-PD-L1 VHH-Fc antibodies. X axis is values of antibody concentration in nanomolar. Y axis is values of NFAT luciferase activity in relative luminescence units. KN035 is a reference anti-h-PD-L1 antibody from AlphaMab. 1F4, 5C12, 6A10, 6D2, 4H8, and 6D1 are anti-h-PD-L1 VHH-Fc clones.

FIG. 2 shows whole cell binding of mouse PD-L1 ELISA positive VHH-Fc antibodies to mouse PD-L1 (m-PD-L1) stably transfected 293T cells as determined by flowcytometry assays. X axis is values of antibody concentration in nanomolar. Y axis is values of Median Fluorescence Intensity of AlexaFlour 488. Atezo (Atezolizumab) is a reference anti-m-PD-L1 antibody from Roche. 5F8, 3H1, 2A11, 1C3, 6D2, and 4H8 are anti-PD-L1 VHH-Fc clones.

FIG. 3A shows the potencies of humanized variant hv7 & hv8 of 5F8 and hv7 of 3H1 in blocking human PD-L1. KN035 is a positive control. The potencies (EC50 in nM) are also shown in the table under the figures. X axis is values of antibody concentration in nanomolar. Y axis is values of NFAT luciferase activity in relative luminescence units.

FIG. 3B shows whole cell binding activities of humanized variants and their parental clones to mouse PD-L1 as determined by flowcytometry using mouse PD-L1 stably transfected 293T cells and anti-human-IgG Fc-AlexaFlour 488 as a secondary antibody. X axis is values of antibody concentration in nanomolar. Y axis is values of Median Fluorescence Intensity. Atezo (Atezolizumab) is a reference control antibody from Roche.

FIG. 3C shows whole cell binding activities of 3H1-hv7 & 5F8-hv8 to mouse PD-L1 as determined in Hepa1-6 cells expressing endogenous m-PD-L1, with anti-human-IgG Fc-AlexaFlour 488 as a secondary antibody. X axis is values of antibody concentration in nanomolar. Y axis is values of Median Fluorescence Intensity.

FIG. 4 shows whole cell binding of selected high affinity PD-L1 clones to endogenous expressed human PD-L1 in T24 cells as determined by flowcytometry assays with anti-human-IgG Fc-AlexaFlour 488 as a secondary antibody. X axis is values of antibody concentration in nanomolar. Y axis is values of Median Fluorescence Intensity of AlexaFlour 488. Atezo (Atezolizumab) is a reference anti-h-PD-L1 antibody from Roche. KN035 is also a reference anti-h-PD-L1 antibody from AlphaMab. 5C12, 1F4, 4D2, 4E2, and 5F8 are lead anti-PD-L1 VHH-Fc clones.

FIG. 5A-5D show SEC-HPLC data using Agilent AdvanceBio SEC 300A 2.7 μm column & Agilent 1200 HPLC. Proteins at concentrations around 1-2 mg/ml were injected with 10 μl at 25 ml/min, the SEC performed in 150 mM Sodium Phosphate buffer. FIG. 5A shows data for 5C12-parental. FIG. 5B shows data for 5C12-NS. FIG. 5C shows data for 5C12-NT. FIG. 5D shows data for 5C12-NQ.

FIG. 6A shows whole cell binding of 5C12 hotspot correct variants to human PD-L1 in h-PD-L1 stably transfected cells as determined by flowcytometry assays using anti-human-IgG Fc-AlexaFlour 488 as a secondary antibody. X axis is values of antibody concentration in nanomolar. Y axis is values of Median Fluorescence Intensity of AlexaFlour 488.

FIG. 6B shows the potencies of 5C12 hotspot corrected variants in blocking human PD-L1 activity as determined using luciferase reporter assays. X axis is values of antibody concentration in nanomolar. Y axis is values of NFAT luciferase activity in relative luminescence units. Atezo (Atezolizumab) is a reference anti-h-PD-L1 antibody from Roche.

FIG. 7A shows whole cell binding of 5C12 humanized variants to human PD-L1 in h-PD-L1 stably transfected cells as determined by flowcytometry assays using anti-human-IgG Fc-AlexaFlour 488 as a secondary antibody (data of selected humanized variants are shown). X axis is values of antibody concentration in nanomolar. Y axis is values of Median Fluorescence Intensity of AlexaFlour 488.

FIG. 7B shows whole cell binding of 5C12 CDR3 hotspot corrected (with N to T) & humanized variants to human PD-L1 in h-PD-L1 stably transfected cells as determined by flowcytometry assays using anti-human-IgG Fc-AlexaFlour 488 as a secondary antibody, data of selected variants shown. X axis is values of antibody concentration in nanomolar. Y axis is values of Median Fluorescence Intensity of AlexaFlour 488. Avelumab is a reference anti-PD-L1 antibody from Pfizer.

FIG. 7C shows whole cell binding of 5C12 CDR3 hotspot corrected (with N to T) & humanized variants to NCI-H441 lung tumor cells as determined by flowcytometry assays using anti-human-IgG Fc-AlexaFlour 488 as a secondary antibody, data of selected variants shown. X axis is values of antibody concentration in nanomolar. Y axis is values of Median Fluorescence Intensity of AlexaFlour 488. Avelumab is a reference anti-PD-L1 antibody from Pfizer.

FIG. 7D shows the potencies of selected 5C12 CDR3 hotspot corrected and humanized variants in blocking human PD-L1 activity was determined using luciferase reporter assays. X axis is values of antibody concentration in nanomolar. Y axis is values of NFAT luciferase activity in relative luminescence units. Atezo (Atezolizumab) is a reference anti-h-PD-L1 antibody from Roche.

FIG. 8A shows whole cell binding of 1F4 CDR3 hotspot corrected and humanized variants to human PD-L1 in h-PD-L1 stably transfected cells as determined by flowcytometry assays using anti-human-IgG Fc-AlexaFlour 488 as a secondary antibody (data of selected humanized variants are shown). X axis is values of antibody concentration in nanomolar. Y axis is values of Median Fluorescence Intensity of AlexaFlour 488. Avelumab is a reference anti-PD-L1 antibody from Pfizer.

FIG. 8B shows the potencies of selected 1F4 CDR3 hotspot corrected and humanized variants in blocking human PD-L1 activity as determined using luciferase reporter assays. X axis is values of antibody concentration in nanomolar. Y axis is values of NFAT luciferase activity in relative luminescence units.

FIG. 9A shows whole cell binding of 4D2 and 4E2 CDR3 hotspot corrected and humanized variants to human PD-L1 in h-PD-L1 stably transfected cells as determined by flowcytometry assays using anti-human-IgG Fc-AlexaFlour 488 as a secondary antibody (data of selected variants shown). X axis is values of antibody concentration in nanomolar. Y axis is values of Median Fluorescence Intensity of AlexaFlour 488. Avelumab is a reference anti-PD-L1 antibody from Pfizer.

FIG. 9B shows whole cell binding of 4D2 and 4E2 CDR3 hotspot corrected and humanized variants to NCI-H441 lung cancer cells as determined by flowcytometry assays using anti-human-IgG Fc-AlexaFlour 488 as a secondary antibody (data of selected variants shown). X axis is values of antibody concentration in nanomolar. Y axis is values of Median Fluorescence Intensity of AlexaFlour 488. Avelumab is a reference anti-PD-L1 antibody from Pfizer.

FIG. 10 shows the potencies of 4D2 and 4E2 CDR3 hotspot corrected and humanized variants in blocking human PD-L1 activity as determined using luciferase reporter assays. X axis is values of antibody concentration in nanomolar. Y axis is values of NFAT luciferase activity in relative luminescence units.

FIG. 11 shows the potencies of lead anti-VEGF clones in blocking human VEGFA-mediated NFAT luciferase activity as determined by luciferase reporter assay using KDR (VEGFR2) and NFAT luciferase reporter gene stably transfected cells. Stable cells in suspension were incubated with serial dilutions of anti-VEGF antibodies in the presence of 50 ng/ml of human VEGFA for 4-5 hours, luciferase activity was determined by adding Bright-Glo luciferase assay buffer with substrate and read by a plate reader. X axis is values of antibody concentration in nanomolar. Y axis is values of NFAT luciferase activity in relative luminescence units.

FIG. 12 shows the potencies of lead anti-VEGF clones in blocking mouse VEGFA-mediated NFAT luciferase activity as determined by luciferase reporter assay using KDR (VEGFR2) and NFAT luciferase reporter gene stably transfected cells. Stable cells in suspension were incubated with serial dilutions of anti-VEGF antibodies in the presence of 50 ng/ml of mouse VEGFA for 4-5 hours, luciferase activity was determined by adding Bright-Glo luciferase assay buffer with substrate and read by a plate reader. X axis is values of antibody concentration in nanomolar. Y axis is values of NFAT luciferase activity in relative luminescence units.

FIG. 13A-13D show schematic diagrams of BsAb constructs in different formats.

FIG. 14A shows potencies of BsAbs in different formats in blocking human PD-L1 activity as determined using luciferase reporter assays. X axis is values of antibody concentration in nanomolar. Y axis is values of NFAT luciferase activity in relative luminescence units.

FIG. 14B shows potencies of BsAbs in different formats in blocking human VEGFA (VEGF165). X axis is values of antibody concentration in nanomolar. Y axis is values of NFAT luciferase activity in relative luminescence units.

FIG. 15A-15F shows double binding ability of BsAbs or monospecific Abs to h-PD-L1 ECD or h-VEGFA as determined by BLI binding assay. Briefly, sensor was loaded with an antibody at 300 nM, then dipped to a solution containing 200 nM of recombinant protein of h-PD-L1 ECD (his-tagged), after 130s association, sensor moved and dipped to another solution containing 200 nM of h-VEGFA (VEGF165, his-tagged). Y axis is shift in nm, X axis is time in second.

FIG. 16A-16E show schematic diagrams of BsAb constructs with different lead anti-PD-L1 clones.

FIG. 17A shows binding abilities of lead BsAbs and their parental bivalent antibodies in whole cell binding to h-PD-L1 endogenously expressed in T24 tumor cells.

FIG. 17B shows potencies of lead BsAbs and their parental bivalent antibodies in blocking human PD-L1 activity as determined using luciferase reporter assays. X axis is values of antibody concentration in nanomolar. Y axis is values of NFAT luciferase activity in relative luminescence units.

FIG. 17C shows potencies of lead BsAbs in blocking human VEGFA (VEGF165) as determined using luciferase reporter assays. X axis is values of antibody concentration in nanomolar. Y axis is values of NFAT luciferase activity in relative luminescence units.

FIG. 18A-18B show schematic diagrams of two lead surrogate BsAb constructs.

FIG. 19A shows potencies of surrogate BsAbs in two different formats in blocking mouse VEGFA (VEGF164) as determined using luciferase reporter assays. X axis is values of antibody concentration in nanomolar. Y axis is values of luciferase activity in relative luminescence units. EC50 values are shown under each figure.

FIG. 19B shows potencies of surrogate BsAbs in two different formats in blocking mouse PD-L1 as determined using luciferase reporter assays. X axis is values of antibody concentration in nanomolar. Y axis is values of luciferase activity in relative luminescence units. EC50 values are shown under each figure.

FIG. 20A shows potencies of lead surrogate BsAb 3B9_Fc_7H3 in blocking mouse PD-L1 as determined using luciferase reporter assays for mouse serum treated samples. X axis is values of antibody concentration in nanomolar. Y axis is values of luciferase activity in relative luminescence units. EC50 values are shown under each figure.

FIG. 20B shows potencies of lead surrogate BsAb 3B9_Fc_7H3 in blocking mouse VEGFA as determined using luciferase reporter assays for mouse serum treated samples. X axis is values of antibody concentration in nanomolar. Y axis is values of luciferase activity in relative luminescence units. EC50 values are shown under each figure.

FIG. 21A shows the tumor volume data from the in vivo efficacy study in the MC38 syngeneic model. X axis is the days of treatment. Y axis is the tumor volume.

FIG. 21B shows the body weight data from the in vivo efficacy study in the MC38 syngeneic model. X axis is the days of treatment. Y axis is the % change of body weight.

FIG. 22 shows the individual tumor volume in each mouse. X axis is the days post treatment. Y axis is the tumor volume.

FIG. 23 shows the comparison of lead BsAbs to IMM2510 in whole cell binding to human PD-L1 stably transfected CHO cells. X axis is values of antibody concentration in nanomolar. Y axis is values of Median Fluorescence Intensity of AlexaFlour 488.

FIG. 24 shows the comparison of lead BsAbs to reference BsAbs in blocking human PD-L1 activity. X axis is values of antibody concentration in nanomolar. Y axis is values of NFAT luciferase activity in relative luminescence units.

FIG. 25 shows the comparison of lead BsAbs to reference BsAbs in blocking human VEGFA. X axis is values of antibody concentration in nanomolar. Y axis is values of NFAT luciferase activity in relative luminescence units.

FIG. 26A shows the tumor volume data from the in vivo efficacy study in the MC38-PD-L1 syngeneic mouse model. X axis is the days of treatment. Y axis is the tumor volume.

FIG. 26B shows the body weight data from the in vivo efficacy study in the MC38-PD-L1 syngeneic mouse model. X axis is the days of treatment. Y axis is the % change of body weight.

FIG. 27 shows the individual tumor volume in each mouse. X axis is the days of treatment. Y axis is the tumor volume.

FIG. 28 lists CDR sequences of the PD-L1 antibodies described in the disclosure.

FIG. 29 lists CDR sequences of the VEGF antibodies described in the disclosure.

FIG. 30 lists amino acid sequences of VHHs as described in the disclosure.

### DETAILED DESCRIPTION

Cancer cells have developed several mechanisms to evade a host's immune surveillance. For example, many tumor or cancer cells express on their surfaces a high level of PD-L1 and PD-L2, both of which bind to PD-1 on the surface of T cells, inducing T cell apoptosis.

In addition, growth of cancer cells depends on sufficient supply of nutrition. Cancer cells themselves can secrete factors that promote blood vessel growth, such as vascular epithelial growth factors (VEGF). Inhibition of the activity of VEGF or its receptors will stop blood supply to a solid tumor, thereby inhibiting its growth.

This disclosure relates to antibodies or antigen-binding fragments, wherein the antibodies or antigen-binding fragments specifically bind to PD-L1 and/or VEGF, or a combination thereof. In some embodiments, the disclosure relates to development of PD-L1/VEGF targeting bispecific antibodies.

### PD-L1

Programmed death ligand 1 (PD-L1 or PDL1) is the principal ligand of programmed death 1 (PD-1), a co-inhibitory receptor that can be constitutively expressed or induced in myeloid, lymphoid, normal epithelial cells and in cancer. Under physiological conditions, the PD-1/PD-L1 interaction is essential in the development of immune tolerance preventing excessive immune cell activity that can lead to tissue destruction and autoimmunity. PD-L1 expression is an immune evasion mechanism exploited by various malignancies and is generally associated with poorer prognosis. PD-L1 expression is also suggested as a predictive biomarker of response to anti-PD-1/PD-L1 therapies; however, contradictory evidence exists as to its role across histotypes. Over the years, anti-PD-1/PD-L1 agents have gained momentum as novel anticancer therapeutics, by inducing durable tumour regression in numerous malignancies including metastatic lung cancer, melanoma and many others.

PD-L1 expression can be constitutive or inducible. Constitutive, low-level PD-L1 expression can be found, on resting lymphocytes, antigen-presenting cells (APCs) and in corneal, syncytiotrophoblastic and Langerhans' islet cells where it contributes to tissue homeostasis in pro-inflammatory responses. PD-L1 confers certain tissues such as placenta, testis and the anterior chamber of the eye an ‘immune privileged’ status, where inoculation of exogenous antigens is tolerated without induction of an inflammatory/immune response.

In carcinogenesis, PD-L1 can be overexpressed as a result of driver oncogenic events. Epidermal growth factor receptor (EGFR) mutations, for instance, positively correlate with PD-L1 expression in lung cancer, with EGFR inhibitors acting as repressors of PD-L1 transcription. In phosphatase and tensin homolog (PTEN)-mutant tumours, PD-L1 overexpression is sustained by unrestrained activation of the PI3K/AKT pathway. In T cell lymphoma, the nucleophosmin (NPM)/anaplastic lymphoma kinase (ALK) fusion gene up-regulates PD-L1 via constitutive STAT3 activation.

The biological functions of PD-L1 depend on binding with PD-1 (CD279), a 288 amino acid long type 1 transmembrane receptor encoded by the PDCD1 gene and physiologically expressed on lymphocytes and myeloid cells. PD-1 is composed of an extracellular IgV-like domain and a transmembrane region. Its intracellular tail is composed of tyrosine based switch motif (ITSM) and immune receptor tyrosine based inhibitory motif sequences. On ligation with PD-L1, recruitment of Src homology 2 domain containing phosphatases 1 and 2 (SHP-1/SHP-2) to the ITSM causes dephosphorylation of signalling kinases such as CD35, PKCO and ZAP70 resulting in a global inhibitory action of T cell expansion. Such inhibitory response is secondary to inactivation of the PI3K-Akt and Ras-MEK-ERK cascades. Casein kinase 2 is a target of SHP-2. Casein kinase 2 (CK-2) dephosphorylation leads to unrestrained activation of PTEN, a physiological PI3K-Akt signalling antagonist. The inhibitory effect of PD-1 on the Ras-MEK-ERK cascade mostly depends on direct inhibition of Ras and dephosphorylation of phospholipase Cy.

A detailed review of PD-L1 and its functions can be found in (1) Kythreotou, Anthousa, et al. “PD-L1.” Journal of clinical pathology 71.3 (2018): 189-194; and (2) Gong, Jun, et al. “Development of PD-1 and PD-L1 inhibitors as a form of cancer immunotherapy: a comprehensive review of registration trials and future considerations.” Journal for immunotherapy of cancer 6.1 (2018): 1-18, each of which is incorporated by reference in its entirety.

### VEGF

VEGF is a pleiotropic growth factor that is central to control of tissue/wound repair programs, and is classified into VEGF-A, VEGF-B, VEGF-C, VEGF-D and PIGF. During the tissue healing process, VEGF simultaneously drives formation of new blood vessels (angiogenesis) while down-regulating immunity (Canic M, et al. The role of vascular endothelial growth factor in wound healing. J Surg Res. 2009, 153:347-358; Leung D W, et al. Vascular endothelial growth factor is a secreted angiogenic mitogen. Science. 1989, 246:1306-1309; Voron T, et al. Control of the immune response by pro-angiogenic factors. Front Oncol. 2014, 4:70). Both of these properties of VEGF are critical to the oncogenic process, as they enable the development of tumor blood vessels and suppress anticancer immunity Manahan D, Weinberg R A. Hallmarks of cancer: the next generation. Cell. 2011, 144:646-674). In particular, VEGF is thought to exert its immune-suppressive effects via three key mechanisms: inhibiting DC maturation, reducing T cell tumor infiltration, and increasing inhibitory cells in tumor microenvironment. Abnormal VEGF expression also contributes to other diseases, including rheumatoid arthritis, diabetic retinopathy, wet form age-related macular degeneration, and glomerular hypertrophy. Avastin, an FDA approved anti-VEGF monoclonal antibody drug, functions to treat cancers (colon cancer, lung cancer) by inhibiting the biological activities of VEGF. Another protein drug targeting VEGF, Aflibercept, was approved in United State and Europe for treatment of wet macular degeneration under tradename Eylea, and for metastatic colorectal cancer as Zaltrap.

The findings that anti-VEGF antibodies decreased the growth of tumor cells implanted in immune-deficient mice opened up translational possibilities for targeting VEGF-VEGFR signaling. In addition, it was also demonstrated that inactivation of a single allele of the vegfa gene in mice resulted in defective vascular development and early embryonic lethality, highlighting the importance of VEGF during embryonic development. Inactivation of both copies of vegfr2 largely pheno-copied vegfa single-allele deletion. The ability to delete VEGF in target tissues with the advent of cre-lox systems created the possibility of assessing the role of VEGF in individual tissues/cells. Numerous studies employing this approach have documented the important role of VEGF in angiogenesis and homeostasis in a variety of pathophysiological circumstances.

VEGF secreted by tumor cells and surrounding stroma stimulates the proliferation and survival of endothelial cells, leading to the formation of new blood vessels, which may be structurally abnormal and leaky. VEGF mRNA is overexpressed in the majority of human tumors and correlates with invasiveness, vascular density, metastasis, recurrence, and prognosis. Several strategies to inhibit the VEGF-VEGFR signaling pathway for the treatment of cancer have been devised.

A detailed review of VEGF and its functions can be found in (1) Apte et al. “VEGF in signaling and disease: beyond discovery and development.” Cell 176.6 (2019): 1248-1264; and (2) Yang et al. “Targeting VEGF/VEGFR to modulate antitumor immunity.” Frontiers in Immunology 9 (2018): 978, each of which is incorporated by reference in its entirety.

### Heavy-Chain Antibody Variable Domain (VHH)

Monoclonal and recombinant antibodies are important tools in medicine and biotechnology. Like all mammals, camelids (e.g., llamas and alpacas) can produce conventional antibodies made of two heavy chains and two light chains bound together with disulfide bonds in a Y shape (e.g., IgG1). However, they also produce two unique subclasses of IgG:IgG2 and IgG3, also known as heavy chain antibody. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allows the structure to be more stable, thus increasing the strength of the interaction.

VHHs offer numerous other advantages compared to conventional antibodies carrying VH and VL domains, including higher stability, solubility, expression yields, and refolding capacity, as well as better in vivo tissue penetration. Moreover, in contrast to the VH domains of conventional antibodies, VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

### Anti-PD-L1 Antibodies and Antigen-Binding Fragments

The disclosure provides e.g., anti-PD-L1 antibodies, the modified antibodies thereof, the chimeric antibodies thereof, and the humanized antibodies thereof. The disclosure also provides variable domains (e.g., VHH) of these antibodies. These VHHs can be used in various multispecific antibody constructs as described herein.

In some embodiments, the CDR sequences for R1-1C3, and R1-1C3 derived antibodies (e.g., humanized antibodies) include CDRs of the VHH domain as set forth in SEQ ID NOs: 96, 97, 98, respectively. In some embodiments, the amino acid sequence for the VHH domain is set forth in SEQ ID NO: 1.

In some embodiments, the CDR sequences for R2-1G4, and R2-1G4 derived antibodies (e.g., humanized antibodies) include CDRs of the VHH domain as set forth in SEQ ID NOs: 99, 100, 101, respectively. In some embodiments, the amino acid sequence for the VHH domain is set forth in SEQ ID NO: 2.

In some embodiments, the CDR sequences for R2-1A3, and R2-1A3 derived antibodies (e.g., humanized antibodies) include CDRs of the VHH domain as set forth in SEQ ID NOs: 102, 103, 104, respectively. In some embodiments, the amino acid sequence for the VHH domain is set forth in SEQ ID NO: 3.

In some embodiments, the CDR sequences for R2-3A5, and R2-3A5 derived antibodies (e.g., humanized antibodies) include CDRs of the VHH domain as set forth in SEQ ID NOs: 99, 100, 101, respectively. In some embodiments, the amino acid sequence for the VHH domain is set forth in SEQ ID NO: 4.

In some embodiments, the CDR sequences for R2-1G10, and R2-1G10 derived antibodies (e.g., humanized antibodies) include CDRs of the VHH domain as set forth in SEQ ID NOs: 96, 97, 98, respectively. In some embodiments, the amino acid sequence for the VHH domain is set forth in SEQ ID NO: 5.

In some embodiments, the CDR sequences for R2-6D2, and R2-6D2 derived antibodies (e.g., humanized antibodies) include CDRs of the VHH domain as set forth in SEQ ID NOs: 105, 106, 107, respectively. In some embodiments, the amino acid sequence for the VHH domain is set forth in SEQ ID NO: 6.

In some embodiments, the CDR sequences for R2-4H8, and R2-4H8 derived antibodies (e.g., humanized antibodies) include CDRs of the VHH domain as set forth in SEQ ID NOs: 108, 109, 110, respectively. In some embodiments, the amino acid sequence for the VHH domain is set forth in SEQ ID NO: 7.

In some embodiments, the CDR sequences for R2-6A10, and R2-6A10 derived antibodies (e.g., humanized antibodies) include CDRs of the VHH domain as set forth in SEQ ID NOs: 111, 112, 113, respectively. In some embodiments, the amino acid sequence for the VHH domain is set forth in SEQ ID NO: 8.

In some embodiments, the CDR sequences for R2-5C12, and R2-5C12 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 9.

In some embodiments, the CDR sequences for R2-1F4, and R2-1F4 derived antibodies (e.g., humanized antibodies) include CDRs of the VHH domain as set forth in SEQ ID NOs: 117, 118, 119, respectively. In some embodiments, the amino acid sequence for the VHH domain is set forth in SEQ ID NO: 10.

In some embodiments, the CDR sequences for R2-4D2, and R2-4D2 derived antibodies (e.g., humanized antibodies) include CDRs of the VHH domain as set forth in SEQ ID NOs: 120, 121, 122, respectively. In some embodiments, the amino acid sequence for the VHH domain is set forth in SEQ ID NO: 11.

In some embodiments, the CDR sequences for R2-4E2, and R2-4E2 derived antibodies (e.g., humanized antibodies) include CDRs of the VHH domain as set forth in SEQ ID NOs: 123, 124, 125, respectively. In some embodiments, the amino acid sequence for the VHH domain is set forth in SEQ ID NO: 12.

In some embodiments, the CDR sequences for R3-3H1, and R3-3H1 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 13.

In some embodiments, the CDR sequences for R3-5F8, and R3-5F8 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 14.

In some embodiments, the CDR sequences for R3-2A11, and R3-2A11 derived antibodies (e.g., humanized antibodies) include CDRs of the VHH domain as set forth in SEQ ID NOs: 99, 100, 101, respectively. In some embodiments, the amino acid sequence for the VHH domain is set forth in SEQ ID NO: 15.

In some embodiments, the CDR sequences for 5F8-Alpaca, and 5F8-Alpaca derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 16.

In some embodiments, the CDR sequences for 5F8-hv1, and 5F8-hv1 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 17.

In some embodiments, the CDR sequences for 5F8-hv2, and 5F8-hv2 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 18.

In some embodiments, the CDR sequences for 5F8-hv3, and 5F8-hv3 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 19.

In some embodiments, the CDR sequences for 5F8-hv4, and 5F8-hv4 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 20.

In some embodiments, the CDR sequences for 5F8-hv5, and 5F8-hv5 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 21.

In some embodiments, the CDR sequences for 5F8-hv6, and 5F8-hv6 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 22.

In some embodiments, the CDR sequences for 5F8-hv7, and 5F8-hv7 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 23.

In some embodiments, the CDR sequences for 5F8-hv8, and 5F8-hv8 derived antibodies (e.g., humanized antibodies) include CDRs of the VHH domain as set forth in SEQ ID NOs: 129, 130, 131, respectively. In some embodiments, the amino acid sequence for the VHH domain is set forth in SEQ ID NO: 24.

In some embodiments, the CDR sequences for 3H1-Alpaca, and 3H1-Alpaca derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 25.

In some embodiments, the CDR sequences for 3H1-hv1, and 3H1-hv1 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 26.

In some embodiments, the CDR sequences for 3H1-hv2, and 3H1-hv2 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 27.

In some embodiments, the CDR sequences for 3H1-hv3, and 3H1-hv3 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 28.

In some embodiments, the CDR sequences for 3H1-hv4, and 3H1-hv4 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 29.

In some embodiments, the CDR sequences for 3H1-hv5, and 3H1-hv5 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 30.

In some embodiments, the CDR sequences for 3H1-hv6, and 3H1-hv6 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 31.

In some embodiments, the CDR sequences for 3H1-hv7, and 3H1-hv7 derived antibodies (e.g., humanized antibodies) include CDRs of the VHH domain as set forth in SEQ ID NOs: 126, 127, 128, respectively. In some embodiments, the amino acid sequence for the VHH domain is set forth in SEQ ID NO: 32.

In some embodiments, the CDR sequences for 3H1-3B9, and 3H1-3B9 derived antibodies (e.g., humanized antibodies) include CDRs of the VHH domain as set forth in SEQ ID NOs: 132, 133, 134, respectively. In some embodiments, the amino acid sequence for the VHH domain is set forth in SEQ ID NO: 33.

In some embodiments, the CDR sequences for 3H1-2A2, and 3H1-2A2 derived antibodies (e.g., humanized antibodies) include CDRs of the VHH domain as set forth in SEQ ID NOs: 111, 112, 113, respectively. In some embodiments, the amino acid sequence for the VHH domain is set forth in SEQ ID NO: 34.

In some embodiments, the CDR sequences for 3H1-2G3, and 3H1-2G3 derived antibodies (e.g., humanized antibodies) include CDRs of the VHH domain as set forth in SEQ ID NOs: 135, 136, 137, respectively. In some embodiments, the amino acid sequence for the VHH domain is set forth in SEQ ID NO: 35.

In some embodiments, the CDR sequences for 3H1-1G9, and 3H1-1G9 derived antibodies (e.g., humanized antibodies) include CDRs of the VHH domain as set forth in SEQ ID NOs: 138, 139, 140, respectively. In some embodiments, the amino acid sequence for the VHH domain is set forth in SEQ ID NO: 36.

In some embodiments, the CDR sequences for 3H1-6D2, and 3H1-6D2 derived antibodies (e.g., humanized antibodies) include CDRs of the VHH domain as set forth in SEQ ID NOs: 141, 142, 143, respectively. In some embodiments, the amino acid sequence for the VHH domain is set forth in SEQ ID NO: 37.

In some embodiments, the CDR sequences for 3H1-2F4, and 3H1-2F4 derived antibodies (e.g., humanized antibodies) include CDRs of the VHH domain as set forth in SEQ ID NOs: 144, 145, 146, respectively. In some embodiments, the amino acid sequence for the VHH domain is set forth in SEQ ID NO: 38.

In some embodiments, the CDR sequences for 5F8-3A3, and 5F8-3A3 derived antibodies (e.g., humanized antibodies) include CDRs of the VHH domain as set forth in SEQ ID NOs: 147, 148, 149, respectively. In some embodiments, the amino acid sequence for the VHH domain is set forth in SEQ ID NO: 39.

In some embodiments, the CDR sequences for 5F8-8E3, and 5F8-8E3 derived antibodies (e.g., humanized antibodies) include CDRs of the VHH domain as set forth in SEQ ID NOs: 150, 151, 152, respectively. In some embodiments, the amino acid sequence for the VHH domain is set forth in SEQ ID NO: 40.

In some embodiments, the CDR sequences for 5F8-8D4, and 5F8-8D4 derived antibodies (e.g., humanized antibodies) include CDRs of the VHH domain as set forth in SEQ ID NOs: 153, 154, 155, respectively. In some embodiments, the amino acid sequence for the VHH domain is set forth in SEQ ID NO: 41.

In some embodiments, the CDR sequences for 5F8-4B12, and 5F8-4B12 derived antibodies (e.g., humanized antibodies) include CDRs of the VHH domain as set forth in SEQ ID NOs: 156, 157, 158, respectively. In some embodiments, the amino acid sequence for the VHH domain is set forth in SEQ ID NO: 42.

In some embodiments, the CDR sequences for 5F8-6D6, and 5F8-6D6 derived antibodies (e.g., humanized antibodies) include CDRs of the VHH domain as set forth in SEQ ID NOs: 159, 160, 161, respectively. In some embodiments, the amino acid sequence for the VHH domain is set forth in SEQ ID NO: 43.

In some embodiments, the CDR sequences for 5F8-8H3, and 5F8-8H3 derived antibodies (e.g., humanized antibodies) include CDRs of the VHH domain as set forth in SEQ ID NOs: 162, 163, 164, respectively. In some embodiments, the amino acid sequence for the VHH domain is set forth in SEQ ID NO: 44.

In some embodiments, the CDR sequences for 5F8-7D5, and 5F8-7D5 derived antibodies (e.g., humanized antibodies) include CDRs of the VHH domain as set forth in SEQ ID NOs: 165, 166, 167, respectively. In some embodiments, the amino acid sequence for the VHH domain is set forth in SEQ ID NO: 45.

In some embodiments, the CDR sequences for 5C12-NQ, and 5C12-NQ derived antibodies (e.g., humanized antibodies) include CDRs of the VHH domain as set forth in SEQ ID NOs: 168, 169, 170, respectively. In some embodiments, the amino acid sequence for the VHH domain is set forth in SEQ ID NO: 46.

In some embodiments, the CDR sequences for 5C12-NT, and 5C12-NT derived antibodies (e.g., humanized antibodies) include CDRs of the VHH domain as set forth in SEQ ID NOs: 171, 172, 173, respectively. In some embodiments, the amino acid sequence for the VHH domain is set forth in SEQ ID NO: 47.

In some embodiments, the CDR sequences for 5C12-NS, and 5C12-NS derived antibodies (e.g., humanized antibodies) include CDRs of the VHH domain as set forth in SEQ ID NOs: 174, 175, 176, respectively. In some embodiments, the amino acid sequence for the VHH domain is set forth in SEQ ID NO: 48.

In some embodiments, the CDR sequences for 5C12-hv1, and 5C12-hv1 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 49.

In some embodiments, the CDR sequences for 5C12-hv1-NQ, and 5C12-hv1-NQ derived antibodies (e.g., humanized antibodies) include CDRs of the VHH domain as set forth in SEQ ID NOs: 168, 169, 170, respectively. In some embodiments, the amino acid sequence for the VHH domain is set forth in SEQ ID NO: 50.

In some embodiments, the CDR sequences for 5C12-hv1-NT, and 5C12-hv1-NT derived antibodies (e.g., humanized antibodies) include CDRs of the VHH domain as set forth in SEQ ID NOs: 171, 172, 173, respectively. In some embodiments, the amino acid sequence for the VHH domain is set forth in SEQ ID NO: 51.

In some embodiments, the CDR sequences for 5C12-hv1-NS, and 5C12-hv1-NS derived antibodies (e.g., humanized antibodies) include CDRs of the VHH domain as set forth in SEQ ID NOs: 174, 175, 176, respectively. In some embodiments, the amino acid sequence for the VHH domain is set forth in SEQ ID NO: 52.

In some embodiments, the CDR sequences for 5C12-hv2, and 5C12-hv2 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 53.

In some embodiments, the CDR sequences for 5C12-hv3, and 5C12-hv3 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 54.

In some embodiments, the CDR sequences for 5C12-hv4, and 5C12-hv4 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 55.

In some embodiments, the CDR sequences for 5C12-hv5, and 5C12-hv5 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 56.

In some embodiments, the CDR sequences for 5C12-hv6, and 5C12-hv6 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 57.

In some embodiments, the CDR sequences for 5C12-hv7, and 5C12-hv7 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 58.

In some embodiments, the CDR sequences for 5C12-hv8, and 5C12-hv8 derived antibodies (e.g., humanized antibodies) include CDRs of the VHH domain as set forth in SEQ ID NOs: 114, 115, 116, respectively. In some embodiments, the amino acid sequence for the VHH domain is set forth in SEQ ID NO: 59.

In some embodiments, the CDR sequences for 5C12-hv7-NT, and 5C12-hv7-NT derived antibodies (e.g., humanized antibodies) include CDRs of the VHH domain as set forth in SEQ ID NOs: 171, 172, 173, respectively. In some embodiments, the amino acid sequence for the VHH domain is set forth in SEQ ID NO: 60.

In some embodiments, the CDR sequences for 5C12-hv9-NT, and 5C12-hv9-NT derived antibodies (e.g., humanized antibodies) include CDRs of the VHH domain as set forth in SEQ ID NOs: 171, 172, 173, respectively. In some embodiments, the amino acid sequence for the VHH domain is set forth in SEQ ID NO: 61.

In some embodiments, the CDR sequences for 1F4-hv1-NT, and 1F4-hv1-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 62.

In some embodiments, the CDR sequences for 1F4-hv2-NT, and 1F4-hv2-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 63.

In some embodiments, the CDR sequences for 1F4-hv3-NT, and 1F4-hv3-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 64.

In some embodiments, the CDR sequences for 1F4-hv4-NT, and 1F4-hv4-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 65.

In some embodiments, the CDR sequences for 1F4-hv5-NT, and 1F4-hv5-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 66.

In some embodiments, the CDR sequences for 1F4-hv6-NT, and 1F4-hv6-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 67.

In some embodiments, the CDR sequences for 1F4-hv7-NT, and 1F4-hv7-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 68.

In some embodiments, the CDR sequences for 1F4-hv8-NT, and 1F4-hv8-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 69.

In some embodiments, the CDR sequences for 1F4-hv9-NT, and 1F4-hv9-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 70.

In some embodiments, the CDR sequences for 1F4-hv10-NT, and 1F4-hv10-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 71.

In some embodiments, the CDR sequences for 1F4-hv11-NT, and 1F4-hv11-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 72.

In some embodiments, the CDR sequences for 1F4-hv12-NT, and 1F4-hv12-NT derived antibodies (e.g., humanized antibodies) include CDRs of the VHH domain as set forth in SEQ ID NOs: 177, 178, 179, respectively. In some embodiments, the amino acid sequence for the VHH domain is set forth in SEQ ID NO: 73.

In some embodiments, the CDR sequences for 4D2-hv1, and 4D2-hv1 derived antibodies (e.g., humanized antibodies) include CDRs of the VHH domain as set forth in SEQ ID NOs: 180, 181, 182, respectively. In some embodiments, the amino acid sequence for the VHH domain is set forth in SEQ ID NO: 74.

In some embodiments, the CDR sequences for 4D2-hv2, and 4D2-hv2 derived antibodies (e.g., humanized antibodies) include CDRs of the VHH domain as set forth in SEQ ID NOs: 180, 181, 182, respectively. In some embodiments, the amino acid sequence for the VHH domain is set forth in SEQ ID NO: 75.

In some embodiments, the CDR sequences for 4D2-hv3, and 4D2-hv3 derived antibodies (e.g., humanized antibodies) include CDRs of the VHH domain as set forth in SEQ ID NOs: 180, 181, 182, respectively. In some embodiments, the amino acid sequence for the VHH domain is set forth in SEQ ID NO: 76.

In some embodiments, the CDR sequences for 4D2-hv4, and 4D2-hv4 derived antibodies (e.g., humanized antibodies) include CDRs of the VHH domain as set forth in SEQ ID NOs: 180, 181, 182, respectively. In some embodiments, the amino acid sequence for the VHH domain is set forth in SEQ ID NO: 77.

In some embodiments, the CDR sequences for 4E2_Alpaca, and 4E2_Alpaca derived antibodies (e.g., humanized antibodies) include CDRs of the VHH domain as set forth in SEQ ID NOs: 183, 184, 185, respectively. In some embodiments, the amino acid sequence for the VHH domain is set forth in SEQ ID NO: 201.

In some embodiments, the CDR sequences for 4E2-hv1, and 4E2-hv1 derived antibodies (e.g., humanized antibodies) include CDRs of the VHH domain as set forth in SEQ ID NOs: 183, 184, 185, respectively. In some embodiments, the amino acid sequence for the VHH domain is set forth in SEQ ID NO: 78.

In some embodiments, the CDR sequences for 4E2-hv2, and 4E2-hv2 derived antibodies (e.g., humanized antibodies) include CDRs of the VHH domain as set forth in SEQ ID NOs: 183, 184, 185, respectively. In some embodiments, the amino acid sequence for the VHH domain is set forth in SEQ ID NO: 79.

In some embodiments, the CDR sequences for 4E2-hv3, and 4E2-hv3 derived antibodies (e.g., humanized antibodies) include CDRs of the VHH domain as set forth in SEQ ID NOs: 183, 184, 185, respectively. In some embodiments, the amino acid sequence for the VHH domain is set forth in SEQ ID NO: 80.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOs: 1-80.

Furthermore, in some embodiments, the antibodies or antigen-binding fragments thereof described herein can also contain one, two, or three VHH domain CDRs selected from the group of SEQ ID NOs: 96-185.

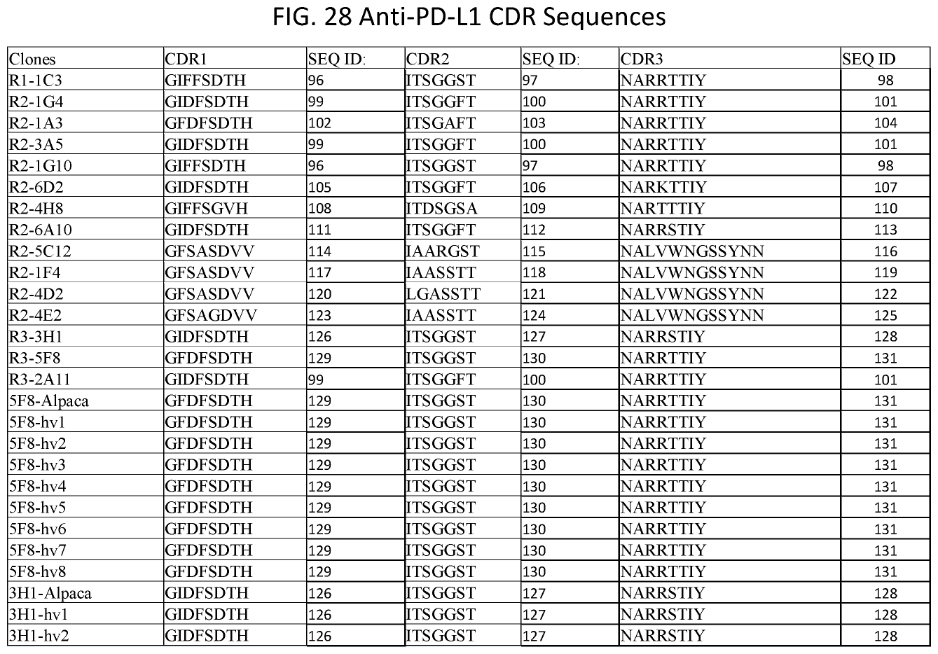

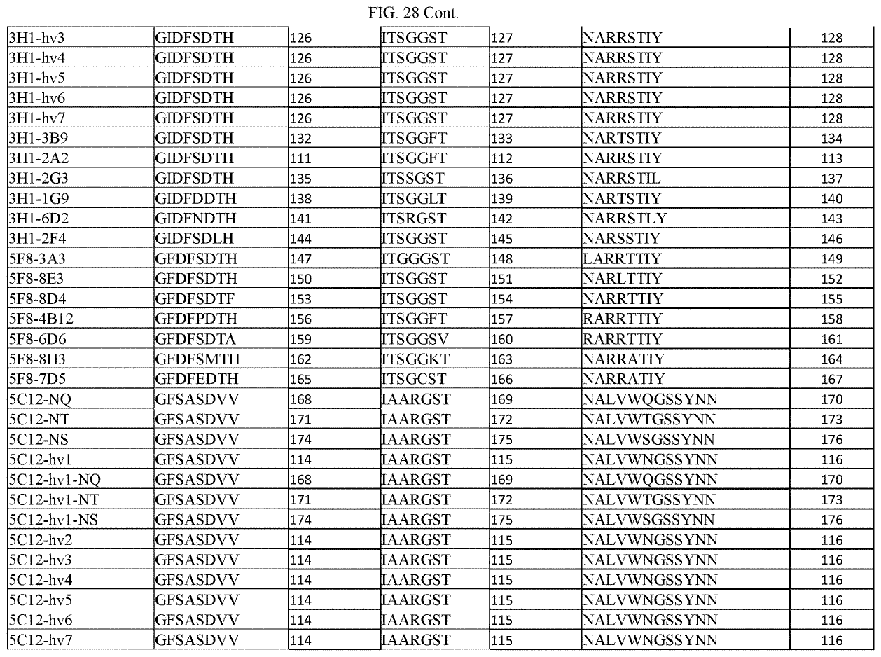

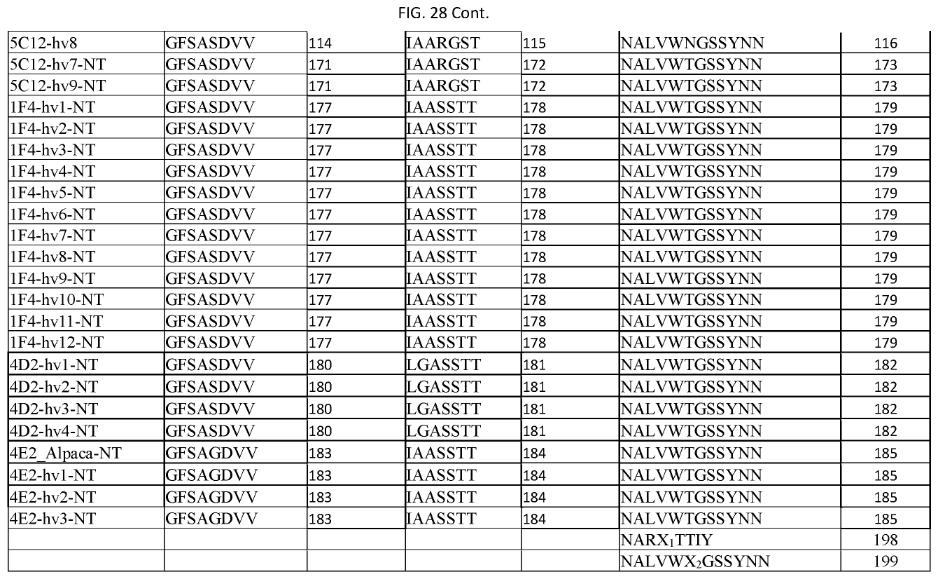

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences are shown in FIG. 28.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 28.

In some embodiments, the antibody or an antigen-binding fragment described herein can contain a heavy-chain antibody variable domain (VHH) containing one, two, or three of the CDRs of SEQ ID NO: 96-185 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on IMGT numbering scheme.

The disclosure also provides antibodies or antigen-binding fragments thereof that bind to PD-L1. The antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is selected from SEQ ID NO: 1-80.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished, e.g., using a Blossum 62 scoring matrix with a gap penalty of 12, a gap extend penalty of 4, and a frameshift gap penalty of 5.

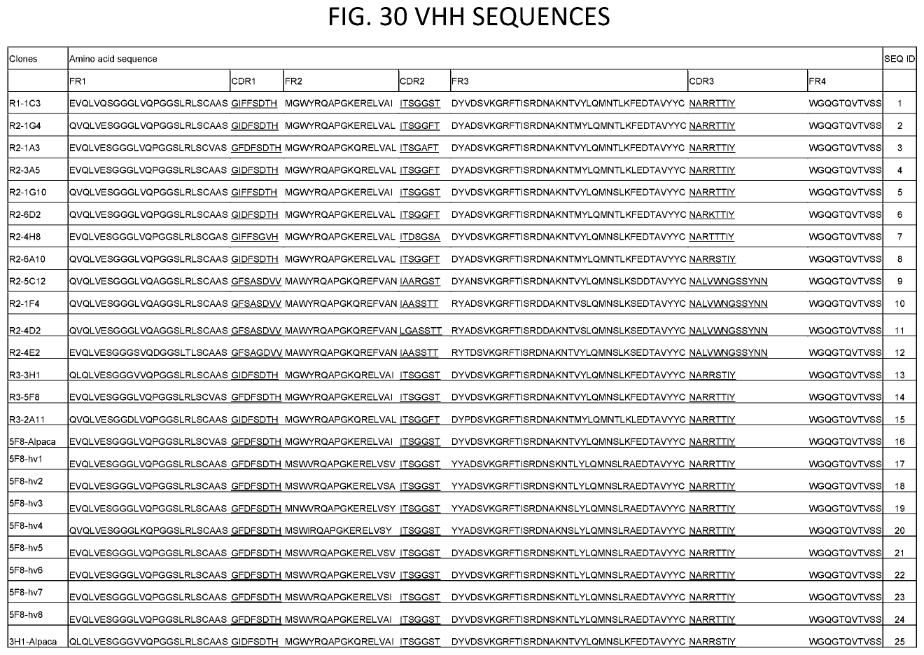

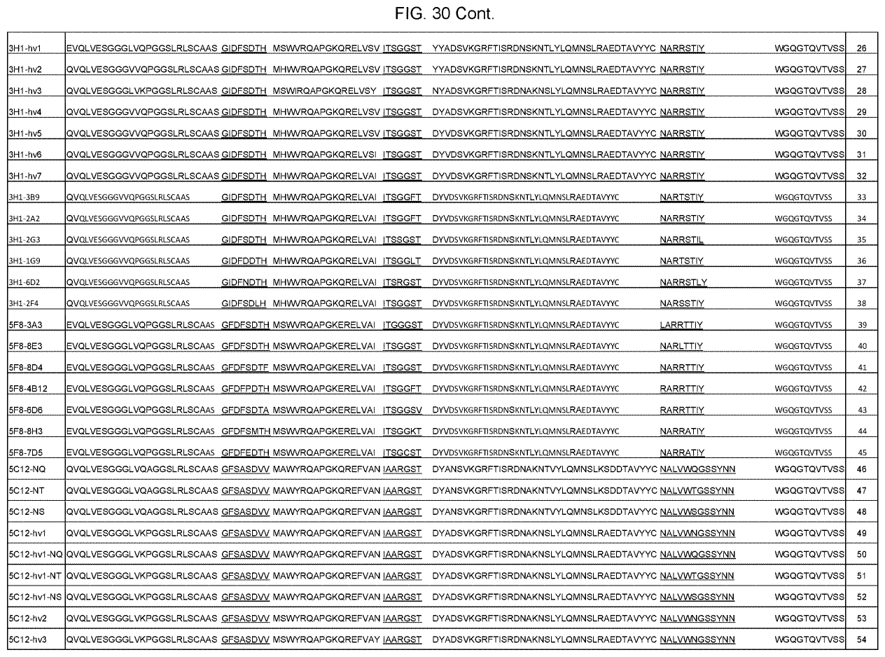

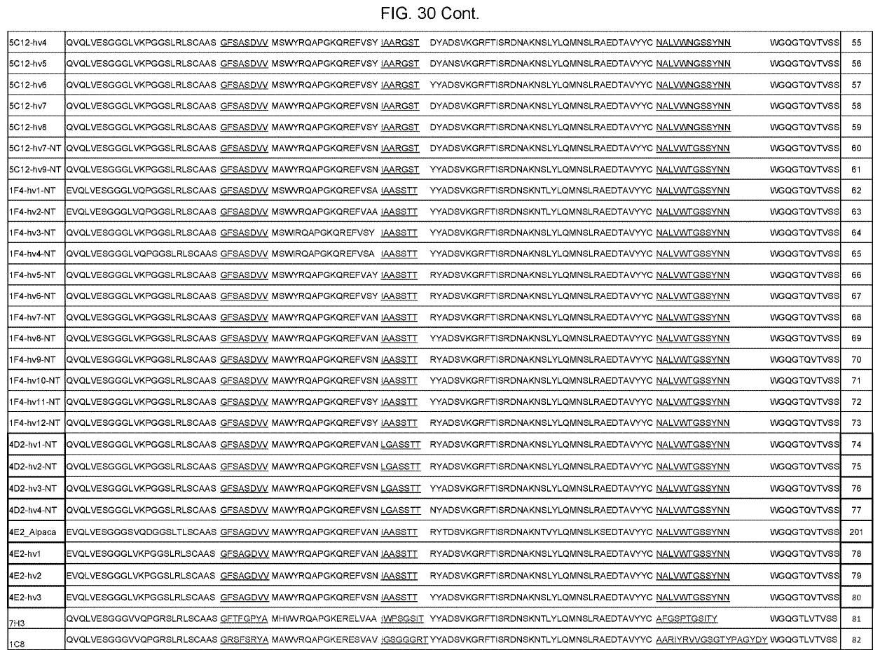

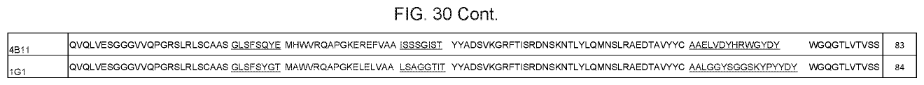

The disclosure also provides nucleic acid comprising a polynucleotide encoding a polypeptide comprising an immunoglobulin heavy-chain antibody variable domain (VHH). The VHH comprises CDRs as shown in FIG. 28, or has sequences as shown in FIG. 30.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

### Anti-VEGF Antibodies and Antigen-Binding Fragments

The disclosure provides e.g., anti-VEGF antibodies, the modified antibodies thereof, the chimeric antibodies thereof, and the humanized antibodies thereof. The disclosure also provides VHH of these antibodies. These VHHs can be used in various multispecific antibody constructs as described herein.

The CDR sequences for 7H3, and 7H3 derived antibodies (e.g., humanized antibodies) include CDRs of the VHH domain as set forth in SEQ ID NOs: 186, 187, and 188, respectively. The amino acid sequence for the VHH domain of 7H3 antibody is set forth in SEQ ID NO: 81.

The CDR sequences for 1C8, and 1C8 derived antibodies (e.g., humanized antibodies) include CDRs of the VHH domain as set forth in SEQ ID NOs: 189, 190, and 191, respectively. The amino acid sequence for the VHH domain of 1C8 antibody is set forth in SEQ ID NO: 82.

The CDR sequences for 4B11, and 4B11 derived antibodies (e.g., humanized antibodies) include CDRs of the VHH domain as set forth in SEQ ID NOs: 192, 193, and 194, respectively. The amino acid sequence for the VHH domain of 4B11 antibody is set forth in SEQ ID NO: 83.

The CDR sequences for 1G1, and 1G1 derived antibodies (e.g., humanized antibodies) include CDRs of the VHH domain as set forth in SEQ ID NOs: 195, 196, and 197, respectively. The amino acid sequence for the VHH domain of 1G1 antibody is set forth in SEQ ID NO: 84.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOs: 81-84.

Furthermore, in some embodiments, the antibodies or antigen-binding fragments thereof described herein can also contain one, two, or three VHH domain CDRs selected from the group of SEQ ID NOs: 186-197.

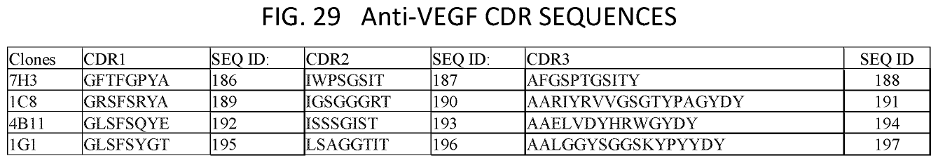

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences are shown in FIG. 29.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 29.

In some embodiments, the antibody or an antigen-binding fragment described herein can contain a heavy-chain antibody variable domain (VHH) containing one, two, or three of the CDRs of SEQ ID NO: 186-197 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on IMGT numbering scheme.

The disclosure also provides antibodies or antigen-binding fragments thereof that bind to VEGF. The antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is selected from SEQ ID NO: 81-84.

The disclosure also provides nucleic acid comprising a polynucleotide encoding a polypeptide comprising an immunoglobulin heavy-chain antibody variable domain (VHH). The VHH comprises CDRs as shown in FIG. 29, or has sequences as shown in FIG. 30.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

### Structures of PD-L1/VEGF Bispecific Antibodies

In some embodiments, the bispecific antibodies are designed to include a VHH that targets PD-L1 and a VHH that targets VEGF. In some embodiments, the present disclosure provides bispecific antibodies that bind to both PD-L1 and VEGF. The bispecific antibodies can be used to treat PD-L1 or VEGF positive cancers (e.g., non-small cell lung cancer) in a subject.

The PD-L1/VEGF bispecific antibodies with specific structures are described below.

### BiSpecific-V1 Structure

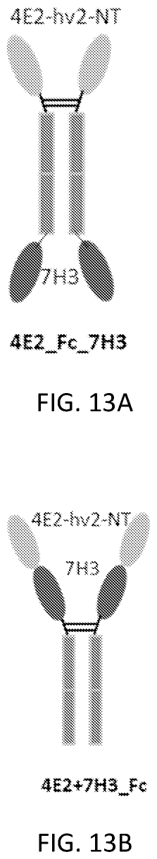

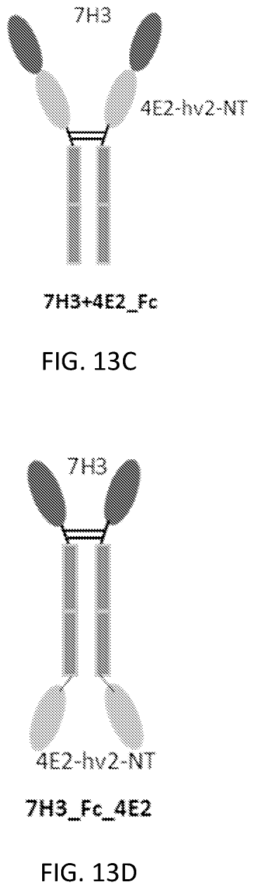

As shown in FIG. 13A, a PD-L1/VEGF bispecific antibody can be prepared to have a BiSpecific-V1 structure. Specifically, the PD-L1/VEGF bispecific antibody comprises (a) a first polypeptide comprising from N-terminus to C-terminus: a first heavy-chain antibody variable domain (VHH1), a first hinge region, a first Fc region, and a third heavy-chain antibody variable domain (VHH3); and (b) a second polypeptide comprising from N-terminus to C-terminus: a second heavy-chain antibody variable region (VHH2), a second hinge region, a second Fc region, and a fourth heavy-chain antibody variable region (VHH4).

In some embodiments, the VHH1 and the VHH2 specifically bind to PD-L1. In some embodiments, the VHH3 and the VHH4 specifically bind to VEGF. In some embodiments, sequences of the VHH1 and the VHH2 are identical. In some embodiments, sequences of the VHH3 and the VHH4 are identical.

In some embodiments, the PD-L1/VEGF bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 85, 89, 90, 91, 92, 93, or 95. In some embodiments, the second polypeptide comprises a sequence that is at least 80%, 85%, 90%, 95%, or 100% identical to SEQ ID NO: 85, 89, 90, 91, 92, 93, or 95.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering.

In some embodiments, the VHH3 is linked to the C-terminus of the first Fc region via a first linker peptide sequence. In some embodiments, the VHH4 is linked to the C-terminus of the second Fc region via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS. In some embodiments, the first and/or second linker peptide sequence comprises a sequence that selected from GSGGSGGSGGSG (SEQ ID NO: 202) and GSGGSGGSGGSGGSG (SEQ ID NO: 203).

### BiSpecific-V2 Structure

As shown in FIG. 13B, a PD-L1/VEGF bispecific antibody can be prepared to have a BiSpecific-V2 structure. Specifically, the PD-L1/VEGF bispecific antibody comprises (a) a first polypeptide comprising from N-terminus to C-terminus: a VHH1, a VHH3, a first hinge region, a first Fc region; and (b) a second polypeptide comprising from N-terminus to C-terminus: a VHH2, a VHH4, a second hinge region, and a second Fc region.

In some embodiments, the VHH1 and the VHH2 specifically bind to PD-L1. In some embodiments, the VHH3 and VHH4 specifically bind to VEGF. In some embodiments, sequences of the VHH1 and the VHH2 are identical. In some embodiments, sequences of the VHH3 and the VHH4 are identical.

In some embodiments, the PD-L1/VEGF bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 86 or 94. In some embodiments, the second polypeptide comprises a sequence that is at least 80%, 85%, 90%, 95%, or 100% identical to SEQ ID NO: 86 or 94.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering.

In some embodiments, the VHH1 is linked to the N-terminus of the VHH3 via a first linker peptide sequence. In some embodiments, the VHH2 is linked to the N-terminus of the VHH4 via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS. In some embodiments, the first and/or second linker peptide sequence comprises a sequence that selected from GSGGSGGSGGSG (SEQ ID NO: 202), and GSGGSGGSGGSGGSG (SEQ ID NO: 203).

### BiSpecific-V3 Structure

As shown in FIG. 13C, a PD-L1/VEGF bispecific antibody can be prepared to have a BiSpecific-V3 structure. Specifically, the PD-L1/VEGF bispecific antibody comprises (a) a first polypeptide comprising from N-terminus to C-terminus: a VHH3, a VHH1, a first hinge region, and a first Fc region; (b) a second polypeptide comprising from N-terminus to C-terminus: a VHH4, a VHH2, a second hinge region, and a second Fc region.

In some embodiments, the VHH1 and the VHH2 specifically bind to PD-L1. In some embodiments, the VHH3 and the VHH4 specifically bind to VEGF. In some embodiments, sequences of the VHH1 and the VHH2 are identical. In some embodiments, sequences of the VHH3 and the VHH4 are identical.

In some embodiments, the first polypeptide comprises a sequence that is at least 80%, 85%, 90%, 95%, or 100% identical to SEQ ID NO: 87. In some embodiments, the second polypeptide comprises a sequence that is at least 80%, 85%, 90%, 95%, or 100% identical to SEQ ID NO: 87.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering.

In some embodiments, the VHH3 is linked to the N-terminus of the VHH1 via a first linker peptide sequence. In some embodiments, the VHH4 is linked to the N-terminus of the VHH2 via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS. In some embodiments, the first and/or second linker peptide sequence comprises a sequence that selected from GSGGSGGSGGSG (SEQ ID NO: 202), and GSGGSGGSGGSGGSG (SEQ ID NO: 203).

### BiSpecific-V4 Structure

As shown in FIG. 13D, a PD-L1/VEGF bispecific antibody can be prepared to have a BiSpecific-V4 structure. Specifically, the PD-L1/VEGF bispecific antibody comprises (a) a first polypeptide comprising from N-terminus to C-terminus: a VHH3, a first hinge region, a first Fc region, and a VHH1; and (b) a second polypeptide comprising from N-terminus to C-terminus: a VHH4, a second hinge region, a second Fc region, and a VHH2.

In some embodiments, the VHH1 and the VHH2 specifically bind to PD-L1. In some embodiments, the VHH3 and the VHH4 specifically bind to VEGF. In some embodiments, sequences of the VHH1 and the VHH2 are identical. In some embodiments, sequences of the VHH3 and the VHH4 are identical.

In some embodiments, the PD-L1/VEGF bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 88. In some embodiments, the second polypeptide comprises a sequence that is at least 80%, 85%, 90%, 95%, or 100% identical to SEQ ID NO: 88.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering.

In some embodiments, the VHH1 is linked to the C-terminus of the first Fc region via a first linker peptide sequence. In some embodiments, the VHH2 is linked to the C-terminus of the second Fc region via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS. In some embodiments, the first and/or second linker peptide sequence comprises a sequence that selected from GSGGSGGSGGSG (SEQ ID NO: 202) and GSGGSGGSGGSGGSG (SEQ ID NO: 203).

### Antibody Characteristics

The anti-PD-L1, anti-VEGF, or anti-PD-L1/VEGF antigen-binding protein construct (e.g., antibodies, bispecific antibodies, trispecific antibodies, multi-specific antibodies, or antibody fragments thereof) can include an antigen binding site that is derived from any anti-PD-L1 antibody, anti-VEGF antibody, or any antigen-binding fragment thereof as described herein.

In some embodiments, the antibodies or antigen-binding fragments thereof described herein are VEGF antagonist. In some embodiments, the antibodies or antigen-binding fragments thereof are VEGF agonist.

In some embodiments, the antibodies, or antigen-binding fragments thereof described herein can bind to PD-L1 and/or VEGF, thereby blocking the interaction of these receptors and their respective ligands; decreasing the phosphorylation of downstream signaling pathways (e.g., ERK and/or Akt pathways); and/or directly killing the cancer cells by ADCC and/or CDC.

In some embodiments, the EC50 of the antibodies or antigen-binding fragments thereof described herein for binding to PD-L1 is less than or about 3 nM, less than or about 2.5 nM, less than or about 2.0 nM, less than or about 1.5 nM, less than or about 1.1 nM, less than or about 1 nM, less than or about 0.8 nM, less than or about 0.7 nM, less than or about 0.6 nM, less than or about 0.5 nM, less than or about 0.4 nM, less than or about 0.3 nM, less than or about 0.2 nM, less than or about 0.1 nM, less than or about 0.09 nM, less than or about 0.08 nM, less than or about 0.05 nM, or less than or about 0.04 nM. In some embodiments, the EC50 of the antibodies or antigen-binding fragments thereof described herein for binding to PD-L1 is about 0.04 nM to 0.1 nM, about 0.05 nM to 0.2 nM, about 0.08 nM to 0.3 nM, about 0.09 nM to 0.4 nM, about 0.1 nM to 0.5 nM, about 0.2 nM to 0.6 nM, about 0.3 nM to 0.7 nM, about 0.4 nM to 0.8 nM, about 0.5 nM to 1 nM, about 0.6 nM to 1.1 nM, or about 0.7 nM to 1.5 nM.

In some embodiments, the antibody (or antigen-binding fragments thereof) specifically binds to an antigen (e.g., PD-L1 or VEGF) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, greater than 2×10⁵/Ms, greater than 3×10⁵/Ms, greater than 4×10⁵/Ms, greater than 5×10⁵/Ms or greater than 6×10⁵/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (Kd-koff/kon). In some embodiments, Kd is less than 1×10⁻⁴ M, less than 1×10⁻⁵ M, less than 1×10⁻⁶ M, less than 1×10⁻⁷M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, Kd is greater than 1×10 4 M, greater than 1×10⁻⁵ M, greater than 1×10⁻⁶M, greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

In some embodiments, the binding affinity to PD-L1 or VEGF is carefully adjusted, e.g., Kd can be between 100 nM~0.1 nM, between 100 nM~1 nM, between 100 nM~10 nM, between 10 nM~0.1 nM, between 10 nM~1 nM, or between 1 nM~0.1 nM.

General techniques for measuring the affinity of an antibody for an antigen include, e.g., ELISA, RIA, and surface plasmon resonance (SPR).

In some embodiments, the stability of the antibodies, or antigen-binding fragments thereof described herein can be analyzed by SEC-HPLC, to assess the amount of aggregation and degradation. In some embodiments, as shown by SEC-HPLC, the high molecular weight (HMW) species constitute less than 20%, 15%, 10%, 7.5%, 5%, 2.5%, 1%, or 0.5%. In some embodiments, as shown by SEC-HPLC, the monomers constitute more than 60%, 65%, 70%, 75%, 80%, 85%, 90%, 97.5%, 95%, 97.5%, or 98%. In some embodiments, as shown by SEC-HPLC, the low molecular weight (LMW) species constitute less than 20%, 15%, 10%, 7.5%, 5%, 2.5%, 1%, or 0.5%. In some embodiments, as shown by SEC-HPLC, the high molecular weight (HMW) species constitute more than 20%, 15%, 10%, 7.5%, 5%, 2.5%, 1%, or 0.5%. In some embodiments, as shown by SEC-HPLC, the monomers constitute less than 60%, 65%, 70%, 75%, 80%, 85%, 90%, 97.5%, 95%, 97.5%, or 98%. In some embodiments, as shown by SEC-HPLC, the low molecular weight (LMW) species constitute more than 20%, 15%, 10%, 7.5%, 5%, 2.5%, 1%, or 0.5%.

In some embodiments, the antibody has a tumor growth inhibition percentage (TG1%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TG1% can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TG1%) is calculated using the following formula:

TG1(%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen binding fragments thereof described herein have the antibody-dependent cell-mediated cytotoxicity (ADCC) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of a reference antibody (e.g., IMM2510). In some embodiments, the antibodies or antigen binding fragments thereof described herein can increase antibody-dependent cell-mediated cytotoxicity (ADCC) by at least 10%, 20%, 30%, 40%, 50%, 60%, 70%, 80%, 90%, 100%, 2 folds, 3 folds, 5 folds, 10 folds, 20 folds, or 100 folds, as compared to that of a non-specific antibody control or an isotype antibody control.

In some embodiments, the antibodies or antigen binding fragments thereof described herein have a cell killing IC50 of less than or about 1000 ng/ml, less than or about 500 ng/ml, less than or about 400 ng/ml, less than or about 300 ng/ml, less than or about 250 ng/ml, less than or about 200 ng/ml, less than or about 100 ng/ml. In some embodiments, the antibodies or antigen binding fragments thereof described herein have a cell killing IC50 value that is less than or about 50%, less than or about 60%, less than or about 70%, less than or about 80%, less than or about 90%, less than or about 100%, less than or about 110%, less than or about 120%, less than or about 130%, less than or about 140%, less than or about 150%, less than or about 200% as compared to that of a reference antibody (e.g., IMM2510). In some embodiments, the antibodies or antigen binding fragments thereof described herein have a cell killing IC50 value that is less than or about 20%, less than or about 10%, less than or about 8%, less than or about 5%, less than or about 3%, less than or about 1% as compared to that of an isotype control antibody.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments can induce apoptosis.

In some embodiments, the antibodies or antigen binding fragments do not have a functional Fc region. For example, the antibodies or antigen binding fragments are Fab, Fab′, F(ab′)2, and Fv fragments.

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides antibodies and antigen-binding fragments thereof that comprise complementary determining regions (CDRs), VHHs, heavy chain variable regions, light chain variable regions, heavy chains, or light chains described herein.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. The IMGT numbering for V-DOMAIN (IG and TR) is derived from the IMGT unique numbering for V-REGION. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); Kontermann, R., & Dübel, S. (Eds.). (2010). Antibody engineering: Volume 2. Springer; Lefranc M.-P., “The IMGT unique numbering for Immunoglobulins, T cell receptors and Ig-like domains” The Immunologist, 7, 132-136 (1999); each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, VHH, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv), or VHH as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies or antigen-binding fragments thereof can bind to two different antigens or two different epitopes. In some embodiments, the antibodies or antigen-binding fragments thereof can bind to three different antigens or three different epitopes.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

In some embodiments, the scFv described herein comprises from N-terminus to C-terminus: VH; the polypeptide linker; and VL. In some embodiments, the scFv described herein comprises from N-terminus to C-terminus: VL; the polypeptide linker; and VH.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Antibodies and antibody fragments of the present disclosure can be modified in the Fc region to provide desired effector functions or serum half-life. In some embodiments, the Fc region in any one of the antibody or antigen-binding fragment described herein comprises an aspartic acid (Asp) at position 239 according to EU numbering. In some embodiments, the Fc region in any one of the antibody or antigen-binding fragment described herein comprises a glutamic acid (Glu) at position 332 according to EU numbering. In some embodiments, the Fc region described herein is any one of the Fc regions described herein, comprising an aspartic acid (Asp) at position 239 according to EU numbering, and/or a glutamic acid (Glu) at position 332 according to EU numbering. In some embodiments, the Asp239 and/or Glu332 described herein can increase effector functions (e.g., ADCC or CDC) of an antibody or antigen binding fragment thereof by at least or about 10%, 20%, 30%, 40%, 50%, 60%, 70%, 80%, 90%, 1-fold, 2-fold, 5-fold, 10-fold, 20-fold, 50-fold, or 100-fold, as compared to those of a wild-type antibody or antigen-binding fragment thereof. Details can be found, e.g., in Lazar, G. A. et al., “Engineered antibody Fc variants with enhanced effector function.” Proceedings of the National Academy of Sciences 103.11 (2006): 4005-4010, which is incorporated herein by reference in its entirety.

In some embodiments, the Fc region in any one of the antibody or antigen-binding fragment described herein comprises a wild-type human IgG1 CH2 domain. In some embodiments, the Fc region in any one of the antibody or antigen-binding fragment described herein comprises a mutated human IgG1 CH2 domain.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multi-specific antibody or antigen-binding fragment thereof described herein (e.g., a PD-L1/VEGF bispecific antibody) binds to an antigen (e.g., PD-L1) with a binding ability that is about 50%, about 60%, about 70%, about 80%, about 90%, about 100%, about 110%, about 120%, about 130%, about 140%, about 150%, or about 200% to that of a heavy-chain antibody (e.g., an anti-PD-L1 heavy-chain antibody) comprising the same VHH of the multi-specific antibody.

In some embodiments, the multi-specific antibody or antigen-binding fragment thereof described herein (e.g., a PD-L1/VEGF bispecific antibody) binds to an antigen (e.g., VEGF) with a binding ability that is about 50%, about 60%, about 70%, about 80%, about 90%, about 100%, about 110%, about 120%, about 130%, about 140%, about 150%, or about 200% to that of an antibody or antigen-binding fragment (e.g., an anti-VEGF heavy-chain antibody) comprising the same VHH targeting VEGF of the multi-specific antibody.

In some embodiments, the bispecific antibody or antigen-binding fragment thereof described herein (e.g., a PD-L1/VEGF bispecific antibody) mediates complement-dependent cytotoxicity (CDC) or ADC to at least or about 1 fold, 2 folds, 3 folds, 4 folds, 5 folds, 6 folds, 7 folds, 8 folds, 9 folds, 10 folds, 11 folds, 12 folds, 13 folds, 14 folds, 15 folds, 16 folds, 17 folds, 18 folds, 19 folds, 20 folds, 30 folds, 40 folds, or 50 folds as compared to that mediated by an isotype control antibody.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259: 1745-1749, and Cohen, 1993, Science, 259: 1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacl and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

### Methods of Making Antibodies

An isolated fragment of human protein (e.g., PD-L1, VEGF, or cancer antigens) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding ability.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding ability for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels, alpaca and llamas), chicken, goats, and rodents (e.g., rats, mice, hamsters and rabbits), including transgenic rodents genetically engineered to produce human antibodies.

Phage display (panning) can be used to optimize antibody sequences with desired binding abilities. In this technique, a gene encoding single chain Fv (comprising VH or VL) or VHH can be inserted into a phage coat protein gene, causing the phage to “display” the scFv or VHH on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding abilities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions Y349C and T366W. The other heavy chain can have one or more the following substitutions E356C, T366S, L368A, and Y407V. Furthermore, a substitution (-ppcpScp—>-ppcpPcp-) can also be introduced at the hinge regions of both substituted IgG. In some embodiments, one heavy chain has a T366Y (knob) substitution, and the other heavy chain has a Y407T (hole) substitution (EU numbering).

One aspect of the present application provides a heteromultimeric (e.g., heterodimeric) protein comprising a first polypeptide comprising a first heavy chain constant domain 3 (CH3) domain and a second polypeptide comprising a second CH3 domain, wherein the first CH3 domain comprises a substitution relative to a wildtype CH3 domain at amino acid position 354 with a bulky hydrophobic amino acid, and/or the second CH3 domain comprises a substitution relative to a wildtype CH3 domain at amino acid position 347 with a negatively charged amino acid, and wherein the amino acid residue numbering is based on EU numbering. In some embodiments, the bulky hydrophobic amino acid at amino acid position 354 forms a hydrophobic interaction with an amino acid residue in the second CH3 domain. In some embodiments, the second CH3 domain comprises a bulky hydrophobic residue at amino acid position 349 (e.g., Y349). In some embodiments, the negatively charged amino acid at amino acid position 347 forms an ionic bond with an amino acid residue in the first CH3 domain. In some embodiments, the first CH3 domain comprises a positively charged residue at amino acid position 360 (e.g., K360). In some embodiments, the first CH3 domain and the second CH3 domain are human CH3 domains. In some embodiments, the first CH3 domain comprises a substitution selected from the group consisting of S354Y, S354F and S354W. In some embodiments, the first CH3 domain comprises S354Y. In some embodiments, the second CH3 domain does not comprise a compensatory substitution (e.g., a substitution at Y349) for the substitution of S354 in the first CH3 domain. In some embodiments, the second CH3 domain comprises a substitution selected from the group consisting of Q347E and Q347D. In some embodiments, the second CH3 domain comprises Q347E. In some embodiments according to any one of the heteromultimeric proteins described above, the first CH3 domain and the second CH3 domain further comprise knob-into-hole (KIH) residues. In some embodiments, the knob-into-hole residues are T366Y and Y407T. In some embodiments, the first CH3 domain comprises T366Y and S354Y, and the second CH3 domain comprises Y407T and Q347E. In some embodiments, the first CH3 domain comprises Y407T and S354Y, and the second CH3 domain comprises T366Y and Q347E. Details can be found, e.g., in PCT/US2020/025469, which is incorporated herein by reference.

### Methods of Treatment

The methods described herein include methods for the treatment of disorders associated with cancer. Generally, the methods include administering a therapeutically effective amount of engineered bispecific antibodies of antigen-binding fragments thereof as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., bispecific antibodies) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth.

The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing PD-L1. In some embodiments, the cancer is a cancer expressing VEGF. In some embodiments, the cancer is a cancer expressing both PD-L1 and VEGF.

In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma, cervical cancer, endometrial carcinoma, or hepatocellular carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck squamous cell carcinoma. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the cancer is colon cancer, rectum cancer, lung cancer, breast cancer, kidney cancer, hepatocellular carcinoma, renal cancer, endometrial carcinoma, pancreatic cancer, head and neck cancer or late-stage solid tumor.

In some embodiments, the cancer cells described herein are cell lines. In some embodiments, the cancer cells have an elevated VEGF or PD-L1 level, e.g., at least 10%, at least 20%, at least 30%, at least 40%, at least 50% higher than non-cancerous cells.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of a disease (e.g., an autoimmune disease or a cancer) in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, antigen-binding fragment, antibody-drug conjugates, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an VEGF inhibitor, a PD-1 inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-VEGFR antibody, an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antibodies, antigen-binding fragments, or antibody-drug conjugates described herein. Two or more (e.g., two, three, or four) of any of the antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50: ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1: Immunization, Generation of Anti-Human PD-L1 VHH Antibodies & Hits Identification

Antigens of recombinant proteins of human, cynomolgus & mouse PD-L1 extra cellular domain (ECD) were purchased from Sinobiological USA. Immunization of PD-L1 antigens was performed using a 2 year's old naïve female alpaca. The titer of serum antibodies was measured by ELISA assays. After 4 rounds of immunization, a high titer (1:100,000) of anti-h-PD-L1 was observed. 80 ml of whole blood was collected from the alpaca, and PBMCs were isolated. RNA was isolated from the PBMCs.

The VHH (a variable domain of the heavy chain of a heavy-chain antibody) genes were amplified with PCR using 10 pairs of specifically designed primers covering all alpaca germlines. The PCR products were purified by DNA agarose gel & gel extraction kit, constructed into a phagemid vector pADL-23c and transformed to TG1 electrocompetent cells by electroporation. Transformed TG1 cells were cultured in 2YT medium and phages displaying target VHHs were produced by adding helper phage and culturing overnight. Phages in supernatants of culture were harvested by precipitation with 4% PGE/0.5M NaCl and high-speed centrifugation. Panning of binders to human PD-L1 (h-PD-L1) or mouse PD-L1 (m-PD-L1) antigen was performed using streptavidin-coupled Dynabeads coated with biotinylated h-PD-L1 ECD protein or m-PD-L1 ECD protein. After 2-3 round of panning, binders of h-PD-L1 or m-PD-L1 were eluted and were used to infect SS320 cells. Colonies of SS320 cells were picked and cultured in 2YT medium and 0.5 mM IPTG was added for secretion of VHH antibodies. Supernatants with VHH antibodies were screened by ELISA assays using h-PD-L1 ECD antigen coated plates, followed by flow cytometry whole cell binding assays using 293T cells stably expressing h-PD-L1. Whole cell binders were picked for sequencing. 54 clones with different sequences were obtained, many of which have only minor sequence differences in CDRs or frameworks. 24 clones with more unique sequences were identified. The CDR and framework sequences were obtained by analysis of sequences using IMGT (the international ImMunoGeneTics information system).

### Example 2: Characterization of Functional PD-L1 VHH Antibodies

24 unique clones were used to make bivalent antibodies with the human IgG1 Fc region (VHH-Fc) by adding hinge region and domains of human IgG1 constant heavy chain 2 (CH2) and constant heavy chain 3 (CH3). Constructed bivalent VHH-Fc antibodies were transiently expressed in CHO cells, and proteins in supernatants were harvested and purified by Protein A resin.

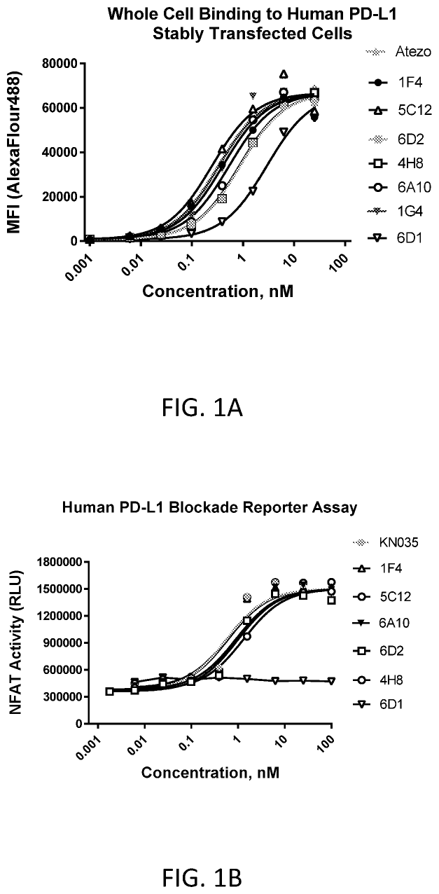

Binding of bivalent PD-L1 VHH-Fc clones to human PD-L1 in stably transfected cells were determined by flowcytometry whole cell binding (WCB) assays. Briefly, antibodies (at different concentrations) were incubated with h-PD-L1 stably expressing cells (0.2×10⁶/ml) in 100 μl/well in 96-well plates in FACS buffer (PBS with 1.0% FBS) for 30 min. After washing, Alexaflour 488 conjugated anti-human IgG Fc secondary antibody (Alexa Fluor® 488 AffiniPure Goat Anti-Human IgG, Fcγ fragment specific, Jackson labs, 1:750 dilution) was added, and incubated for 30 min. After washing, Median Fluorescence Intensity was measured using CytoFlex (Beckman Coulter) by gating live cell population and using the FITC channel. EC50 of binding was calculated using GraphPad Prism7.0. Representative results are shown in FIG. 1A. Summary of functional binders (all fused with human IgG1 Fc) shown in the Table 1 below.

Function of PD-L1 VHH-Fc clones was determined by PD-L1 blockade assay. Briefly, h-PD-L1 stably expressing CHO cells were cultured overnight at 20 k/well in 96-well half-area cell culture plate. Culture medium was removed, and serial dilutions of PD-L1 antibodies were added at 20 μl/well. Human PD1 & NFAT reporter gene stably transfected Jurkat cells were added at a density of 2 million/ml at 20 μl/well. After incubating for 5 hours, 40 μl of Bright-Glo luciferase assay buffer with substrate (Promega) was added and luciferase activity was measured by chemiluminescence activity using a plate reader EnSight (Perkin Elmer). The antibody's potency in blocking PD-L1 activity was calculated using non-linear regression by GraphPad Prims7.0. Representative results are shown in FIG. 1B. The summary of potencies (EC50 values in nM) of functional clones are shown in the table below. R1-R3 (clone names) stand for panning round 1 to 3.

TABLE 1
Summary of EC50 in whole cell binding assay and PD-L1 blocking assay.

| | | EC50, nM in WCB to | EC50, nM in PD-L1 Blockade |
|---|---|---|---|
| | Clone Names | h-PD-L1 | Reporter Assay |
| 1 | R1-1C3 | 1.69 | 1.14 |
| 2 | R2-1G4 | 0.42 | 0.58 |
| 3 | R2-1A3 | 0.88 | 0.76 |
| 4 | R2-3A5 | 0.6 | 0.72 |
| 5 | R2-1G10 | 0.63 | 0.85 |
| 6 | R2-6D2 | 0.93 | 1.14 |
| 7 | R2-4H8 | 0.87 | 1.59 |
| 8 | R2-6A10 | 0.54 | 1.20 |
| 9 | R2-5C12 | 0.24 | 0.77 |
| 10 | R2-1F4 | 0.22 | 0.76 |
| 11 | R2-4D2 | 0.202 | 1.12 |
| 12 | R2-4E2 | 0.148 | 1.18 |
| 13 | R3-3H1 | 0.815 | 1.88 |
| 14 | R3-5F8 | 0.53 | 1.00 |
| 15 | R3-2A11 | 1.16 | 3.00 |

Sequences of functional binders are shown in FIGS. 28 and 30, and the table below.

TABLE 2
Sequences of CDRs and Frameworks for functional binders

| | | SEQ |
|---|---|---|
| Clones | FR1 CDR1 FR2 CDR2 FR3 CDR3 FR4 | ID |
| R1-1C3 | EVQLVQSGGGLVQPGGSLRLSCAASGIFFSDTHMGWYRQAPGKERELVAIITS | 1 |
| | GGSTDYVDSVKGRFTISRDNAKNTVYLQMNTLKFEDTAVYYCNARRTTIYW | |
| | GQGTQVTVSS | |
| R2-1G4 | QVQLVESGGGLVQPGGSLRLSCAASGIDFSDTHMGWYRQAPGKERELVALIT | 2 |
| | SGGFTDYADSVKGRFTISRDNAKNTMYLQMNTLKFEDTAVYYCNARRTTIY | |
| | WGQGTQVTVSS | |
| R2-1A3 | EVQLVESGGGLVQPGGSLRLSCVASGFDFSDTHMGWYRQAPGKQRELVALIT | 3 |
| | SGAFTDYADSVKGRFTISRDNAKNTVYLQMNTLKFEDTAVYYCNARRTTIYW | |
| | GQGTQVTVSS | |
| R2-3A5 | EVQLVESGGGLVQPGGSLRLSCAASGIDFSDTHMGWYRQAPGKQRELVALIT | 4 |
| | SGGFTDYADSVKGRFTISRDNAKNTMYLQMNTLKLEDTAVYYCNARRTTIY | |
| | WGQGTQVTVSS | |
| R2- | QVQLVESGGGLVQPGGSLRLSCAASGIFFSDTHMGWYRQAPGKERELVAIITS | 5 |
| 1G10 | GGSTDYVDSVKGRFTISRDNAKNTVYLQMNSLKFEDTAVYYCNARRTTIYWG | |
| | QGTQVTVSS | |
| R2-6D2 | QVQLVESGGGLVQPGGSLRLSCAASGIDFSDTHMGWYRQAPGKERELVALIT | 6 |
| | SGGFTDYADSVKGRFTISRDNAKNTMYLQMNTLKFEDTAVYYCNARKTTIY | |
| | WGQGTQVTVSS | |
| R2-4H8 | EVQLVESGGGLVQPGGSLRLSCGASGIFFSGVHMGWYRQAPGKERELVALIT | 7 |
| | DSGSADYVDSVKGRFTISRDNAKNTVYLQMNSLKFEDTAVYYCNARTTTIYW | |
| | GQGTQVTVSS | |
| R2- | QVQLVESGGGLVQPGGSLRLSCAASGIDFSDTHMGWYRQAPGKERELVALIT | 8 |
| 6A10 | SGGFTDYADSVKGRFTISRDNAKNTMYLQMNTLKFEDTAVYYCNARRSTIYW | |
| | GQGTQVTVSS | |
| R2-5C12 | QVQLVESGGGLVQAGGSLRLSCAASGFSASDVVMAWYRQAPGKQREFVANI | 9 |
| | AARGSTDYANSVKGRFTISRDNAKNTVYLQMNSLKSDDTAVYYCNALVWNG | |
| | SSYNNWGQGTQVTVSS | |
| R2-1F4 | QVQLVESGGGLVQAGGSLRLSCAASGFSASDVVMAWYRQAPGKQREFVANI | 10 |
| | AASSTTRYADSVKGRFTISRDDAKNTVSLQMNSLKSEDTAVYYCNALVWNGS | |
| | SYNNWGQGTQVTVSS | |
| R2-4D2 | QVQLVESGGGLVQAGGSLRLSCAASGFSASDVVMAWYRQAPGKQREFVANL | 11 |
| | GASSTTRYADSVKGRFTISRDDAKNTVSLQMNSLKSEDTAVYYCNALVWNGS | |
| | SYNNWGQGTQVTVSS | |
| R2-4E2 | EVQLVESGGGSVQDGGSLTLSCAASGFSAGDVVMAWYRQAPGKQREFVANI | 12 |
| | AASSTTRYTDSVKGRFTISRDNAKNTVYLQMNSLKSEDTAVYYCNALVWNGS | |
| | SYNNWGQGTQVTVSS | |
| R3-3H1 | QLQLVESGGGVVQPGGSLRLSCAASGIDFSDTHMGWYRQAPGKQRELVAIITS | 13 |
| | GGSTDYVDSVKGRFTISRDNAKNTVYLQMNSLKFEDTAVYYCNARRSTIYWG | |
| | QGTQVTVSS | |
| R3-5F8 | EVQLVESGGGLVQPGGSLRLSCVASGFDFSDTHMGWYRQAPGKERELVAIITS | 14 |
| | GGSTDYVDSVKGRFTISRDNAKNTVYLQMNSLKFEDTAVYYCNARRTTIYWG | |
| | QGTQVTVSS | |
| R3- | QVQLVESGGDLVQPGGSLRLSCAASGIDFSDTHMGWYRQAPGKQRELVALIT | 15 |
| 2A11 | SGGFTDYPDSVKGRFTISRDNAKNTMYLQMNTLKLEDTAVYYCNARRTTIYW | |
| | GQGTQVTVSS | |

### Example 3: Characterization and Selection of Lead Mouse PD-L1 Binders

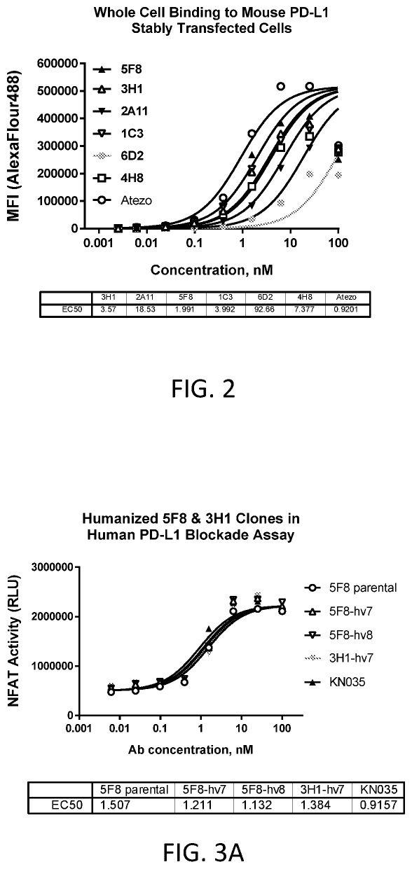

Affinities of antibodies (all fused with human IgG1 Fc) to human PD-L1 were determined by BLI (Bio-layer Interferometry) kinetic assay using a Gator instrument and the recombinant protein of human PD-L1-ECD. High affinity binders are shown in the table below. 5F8 has the highest affinity, as determined by equilibrium dissociation constant (KD). The cross-binding of PD-L1 clones to mouse PD-L1 was determined by ELISA assays using mouse PD-L1-ECD antigen coated plates, followed by whole cell binding to mouse PD-L1 stably transfected 293T cells. Positive mouse PD-L1 binders are shown in FIG. 2.

TABLE 3
Affinities of top PD-L1 clones in kinetic binding assay

| Clones | koff(1/s) | kon(1/Ms) | KD(M) |
|---|---|---|---|
| 5F8 | 8.65E−06 | 1.60E+05 | 5.40E−11 |
| 3H1 | 1.39E−04 | 1.15E+05 | 1.20E−9 |
| 1F4 | 7.79E−04 | 3.83E+05 | 2.04E−09 |
| 5C12 | 1.54E−03 | 3.08E+05 | 5.01E−09 |
| Atezo | 8.01E−04 | 4.12E+05 | 1.94E−09 |
| KN035 | 8.82E−04 | 4.12E+05 | 2.14E−09 |

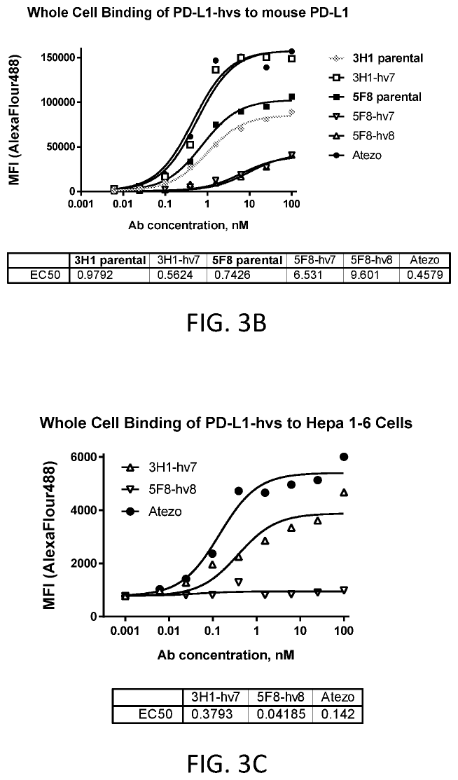

Clones 5F8 & 3H1 were selected for humanization based on their (1) binding affinities to human PD-L1 and (2) EC50 of whole cell binding to mouse PD-L1 (FIG. 2). Humanization was performed by analysis of germline sequence using Igblast. The closest germline sequences were used to change the frameworks of 5F8 and 3H1. Eight humanized variants were made for clone 5F8, and 7 humanized variants were made for clone 3H1, all fused with human IgG1 Fc. Variant 7 and 8 of 5F8 (5F8-hv7 & 8) and variant 7 of 3H1 (3H1-hv7) retained h-PD-L1 blockade function (FIG. 3A), with similar potency to the reference antibody Envafolimab (KN035), a single-domain PD-L1 antibody. To select a potent mouse PD-L1 binder from these humanized variants, whole cell binding assay was performed using 293T mouse PD-L1 stably transfected cells. Binding activities were shown in FIG. 3B. Among humanized variants, 3H1-hv7 showed similar binding ability as Atezolizumab to mouse PD-L1. All other variants showed much weaker binding abilities (data not shown). Potent human PD-L1 blockers 5F8-hv7 and hv8 showed much lower binding abilities to mouse PD-L1 in mouse PD-L1 overexpressed 293T cells (FIG. 3B). Binding of 3H1-hv7 and 5F8-hv8 to endogenously expressed mouse PD-L1 in mouse cancer cells were further examined using Hepa1-6 cells. As shown in FIG. 3C, surprisingly, clone 3H1-hv7 showed much weaker binding ability to endogenously expressed mouse PD-L1, and clone 5F8-hv8 showed no binding ability. The sequences of humanized variants and the parental clones 5F8 and 3H1 are shown in FIGS. 28 and 30, and the tables below.

TABLE 4
Sequences of R3-5F8 humanized variants

| Clone | | SEQ |
|---|---|---|
| names | FR1CDR1FR2CDR2FR3CDR3FR4 | ID |
| 5F8- | EVQLVESGGGLVQPGGSLRLSCVASGFDFSDTHMGWYRQAPGKERELVAIITSG | 16 |
| Alpaca | GSTDYVDSVKGRFTISRDNAKNTVYLQMNSLKFEDTAVYYCNARRTTIYWGQG | |
| | TQVTVSS | |
| 5F8-hv1 | EVQLVESGGGLVQPGGSLRLSCAASGFDFSDTHMSWVRQAPGKERELVSVITSG | 17 |
| | GSTYYADSVKGRFTISRDNSKNTLYLQMNSLRAEDTAVYYCNARRTTIYWGQG | |
| | TQVTVSS | |
| 5F8-hv2 | EVQLVESGGGLVQPGGSLRLSCAASGFDFSDTHMSWVRQAPGKERELVSAITSG | 18 |
| | GSTYYADSVKGRFTISRDNSKNTLYLQMNSLRAEDTAVYYCNARRTTIYWGQG | |
| | TQVTVSS | |
| 5F8-hv3 | EVQLVESGGGLVQPGGSLRLSCAASGFDFSDTHMNWVRQAPGKERELVSYITSG | 19 |
| | GSTYYADSVKGRFTISRDNAKNSLYLQMNSLRAEDTAVYYCNARRTTIYWGQG | |
| | TQVTVSS | |
| 5F8-hv4 | QVQLVESGGGLKQPGGSLRLSCAASGFDFSDTHMSWIRQAPGKERELVSYITSG | 20 |
| | GSTYYADSVKGRFTISRDNAKNSLYLQMNSLRAEDTAVYYCNARRTTIYWGQG | |
| | TQVTVSS | |
| 5F8-hv5 | EVQLVESGGGLVQPGGSLRLSCAASGFDFSDTHMSWVRQAPGKERELVSVITSG | 21 |
| | GSTDYADSVKGRFTISRDNSKNTLYLQMNSLRAEDTAVYYCNARRTTIYWGQG | |
| | TQVTVSS | |
| 5F8-hv6 | EVQLVESGGGLVQPGGSLRLSCAASGFDFSDTHMSWVRQAPGKERELVSVITSG | 22 |
| | GSTDYVDSVKGRFTISRDNSKNTLYLQMNSLRAEDTAVYYCNARRTTIYWGQG | |
| | TQVTVSS | |
| 5F8-hv7 | EVQLVESGGGLVQPGGSLRLSCAASGFDFSDTHMSWVRQAPGKERELVSIITSG | 23 |
| | GSTDYVDSVKGRFTISRDNSKNTLYLQMNSLRAEDTAVYYCNARRTTIYWGQG | |
| | TQVTVSS | |
| 5F8-hv8 | EVQLVESGGGLVQPGGSLRLSCAASGFDFSDTHMSWVRQAPGKERELVAIITSG | 24 |
| | GSTDYVDSVKGRFTISRDNSKNTLYLQMNSLRAEDTAVYYCNARRTTIYWGQG | |
| | TQVTVSS | |

TABLE 5
Sequences of R3-3H1 humanized variants

| Clone | | SEQ |
|---|---|---|
| names | FR1CDR1FR2CDR2FR3CDR3FR4 | ID |
| 3H1- | QLQLVESGGGVVQPGGSLRLSCAASGIDFSDTHMGWYRQAPGKQRELVAIIT | 25 |
| Alpaca | SGGSTDYVDSVKGRFTISRDNAKNTVYLQMNSLKFEDTAVYYCNARRSTIY | |
| | WGQGTQVTVSS | |
| 3H1-hv1 | EVQLVESGGGLVQPGGSLRLSCAASGIDFSDTHMSWVRQAPGKQRELVSVIT | 26 |
| | SGGSTYYADSVKGRFTISRDNSKNTLYLQMNSLRAEDTAVYYCNARRSTIY | |
| | WGQGTQVTVSS | |
| 3H1-hv2 | QVQLVESGGGVVQPGGSLRLSCAASGIDFSDTHMHWVRQAPGKQRELVSVI | 27 |
| | TSGGSTYYADSVKGRFTISRDNSKNTLYLQMNSLRAEDTAVYYCNARRSTIY | |
| | WGQGTQVTVSS | |
| 3H1-hv3 | QVQLVESGGGLVKPGGSLRLSCAASGIDFSDTHMSWIRQAPGKQRELVSYITS | 28 |
| | GGSTNYADSVKGRFTISRDNAKNSLYLQMNSLRAEDTAVYYCNARRSTIYW | |
| | GQGTQVTVSS | |
| 3H1-hv4 | QVQLVESGGGVVQPGGSLRLSCAASGIDFSDTHMHWVRQAPGKQRELVSVI | 29 |
| | TSGGSTDYADSVKGRFTISRDNSKNTLYLQMNSLRAEDTAVYYCNARRSTIY | |
| | WGQGTQVTVSS | |
| 3H1-hv5 | QVQLVESGGGVVQPGGSLRLSCAASGIDFSDTHMHWVRQAPGKQRELVSVI | 30 |
| | TSGGSTDYVDSVKGRFTISRDNSKNTLYLQMNSLRAEDTAVYYCNARRSTIY | |
| | WGQGTQVTVSS | |
| 3H1-hv6 | QVQLVESGGGVVQPGGSLRLSCAASGIDFSDTHMHWVRQAPGKQRELVSIIT | 31 |
| | SGGSTDYVDSVKGRFTISRDNSKNTLYLQMNSLRAEDTAVYYCNARRSTIY | |
| | WGQGTQVTVSS | |
| 3H1-hv7 | QVQLVESGGGVVQPGGSLRLSCAASGIDFSDTHMHWVRQAPGKQRELVAIIT | 32 |
| | SGGSTDYVDSVKGRFTISRDNSKNTLYLQMNSLRAEDTAVYYCNARRSTIY | |
| | WGQGTQVTVSS | |

To increase the binding of 3H1-hv7 to mouse PD-L1, affinity maturation was performed. Primers to make single mutation of amino acid for each CDR region were designed. A library was prepared using assembly PCR and cloning into phagemil vector pADL-23c. Phages were produced using helper phage and phage panning was performed using streptavidin-coupled Dynabeads coated with biotinylated mouse PD-L1-ECD. After two rounds of panning, elution of panning products was used to infect SS320 cells. Colonies were picked and cultured in 2YT medium with 0.5 mM IPTG. Binding activities of VHH antibodies in supernatants were determined by whole cell binding assay using mouse PD-L1 stably transfected 293T cells. Binders with high binding ability were selected for sequencing. Genes of clones with unique sequences were synthesized and cloned into pcDNA3.4 expression vectors fused with human IgG1 Fc. Binding of VHH-Fc clones to mouse PD-L1 was tested in whole cell binding assays using mouse PD-L1 stably expressed 293T cells or Hepa1-6 cells, a mouse cell line with endogenous PD-L1 expression (Table 6). Potency of VHH-Fc clones in blocking mouse PD-L1 activity was also tested in mouse PD-L1 blockade reporter assay. Binders with high binding ability and potency were selected. EC50 in whole cell binding and potency in blocking m-PD-L1 are shown in the table below.

TABLE 6
Whole cell binding ability and potency for
selected 3H1-hv7 affinity matured clones

| | EC50, nM in WCB | EC50, nM in WCB | EC50, nM in m-PD- |
|---|---|---|---|
| Clone names | to 293T m-PD-L1 | to Hepa1-6 cells | L1 Blockade Assay |
| 3H1-3B9 | 0.97 | 0.30 | 0.457 |
| 3H1-2A2 | 1.42 | 0.31 | 0.382 |
| 3H1-2G3 | 1.57 | 0.31 | 0.333 |
| 3H1-1G9 | 1.01 | 0.55 | 0.436 |
| 3H1-6D2 | 1.29 | 0.51 | 0.314 |
| 3H1-2F4 | 1 | 0.65 | 0.825 |
| 3H1-hv7 | 0.56 | 6.78 | 2.141 |
| Atezo | 0.78 | 0.31 | 0.178 |

Sequences are listed in FIGS. 28 and 30, and the table below. Top clone 3B9 was selected as a surrogate molecule to build bispecific antibodies for in vivo study based on binding ability and potency.

TABLE 7
Sequences of selected 3H1-hv7 affinity matured clones

| Clone | | SEQ |
|---|---|---|
| names | FR1CDR1FR2CDR2FR3CDR3FR4 | ID |
| 3H1-hv7 | QVQLVESGGGVVQPGGSLRLSCAASGIDFSDTHMHWVRQAPGKQRELVAIIT | 32 |
| | SGGSTDYVDSVKGRFTISRDNSKNTLYLQMNSLRAEDTAVYYCNARRSTIY | |
| | WGQGTQVTVSS | |
| 3H1-3B9 | QVQLVESGGGVVQPGGSLRLSCAASGIDFSDTHMHWVRQAPGKQRELVAIIT | 33 |
| | SGGFTDYVDSVKGRFTISRDNSKNTLYLQMNSLRAEDTAVYYCNARTSTIYW | |
| | GQGTQVTVSS | |
| 3H1-2A2 | QVQLVESGGGVVQPGGSLRLSCAASGIDFSDTHMHWVRQAPGKQRELVAIIT | 34 |
| | SGGFTDYVDSVKGRFTISRDNSKNTLYLQMNSLRAEDTAVYYCNARRSTIY | |
| | WGQGTQVTVSS | |
| 3H1-2G3 | QVQLVESGGGVVQPGGSLRLSCAASGIDFSDTHMHWVRQAPGKQRELVAIIT | 35 |
| | SSGSTDYVDSVKGRFTISRDNSKNTLYLQMNSLRAEDTAVYYCNARRSTILW | |
| | GQGTQVTVSS | |
| 3H1-1G9 | QVQLVESGGGVVQPGGSLRLSCAASGIDFDDTHMHWVRQAPGKQRELVAII | 36 |
| | TSGGLTDYVDSVKGRFTISRDNSKNTLYLQMNSLRAEDTAVYYCNARTSTIY | |
| | WGQGTQVTVSS | |
| 3H1-6D2 | QVQLVESGGGVVQPGGSLRLSCAASGIDFNDTHMHWVRQAPGKQRELVAII | 37 |
| | TSRGSTDYVDSVKGRFTISRDNSKNTLYLQMNSLRAEDTAVYYCNARRSTLY | |
| | WGQGTQVTVSS | |
| 3H1-2F4 | QVQLVESGGGVVQPGGSLRLSCAASGIDFSDLHMHWVRQAPGKQRELVAIIT | 38 |
| | SGGSTDYVDSVKGRFTISRDNSKNTLYLQMNSLRAEDTAVYYCNARSSTIYW | |
| | GQGTQVTVSS | |

Affinity maturation for the 5F8-hv8 clone was also performed and clones (all fused with human IgG1 Fc) with increased potency in blocking human PD-L1 are shown in the table below.

TABLE 8
Whole cell binding ability and potency for
selected 5F8-hv8 affinity matured clones

| | EC50 (nM) | EC50 (nM) |
|---|---|---|
| Clone name | in h-PD-L1 WCB | in h-PD-L1 Blockade Assay |
| 5F8-6D6 | 0.52 | 1.38 |
| 5F8-8D4 | 0.49 | 1.84 |
| 5F8-4B12 | 0.63 | 1.59 |
| 5F8-3A3 | 0.73 | 1.38 |
| 5F8-8E3 | 0.57 | 1.44 |
| 5F8-8H3 | 0.51 | 1.60 |
| 5F8-7D5 | 0.51 | 1.58 |
| 5F8-hv8 parental | 0.69 | 1.85 |
| Atezo | 0.20 | 1.61 |

The sequences are shown in FIGS. 28 and 30, and the table below.

TABLE 9
Sequences of selected 5F8-hv8 affinity matured clones

| Clones | FR1CDR1FR2CDR2FR3CDR3FR4 | SEQ ID |
|---|---|---|
| 5F8-hv8 | EVQLVESGGGLVQPGGSLRLSCAASITSGGSTMSWVRQAPGKERELVAIITSGGSTDY | 24 |
| | VDSVKGRFTISRDNSKNTLYLQMNSLRAEDTAVYYCNARRTTIYWGQGTQVTVSS | |
| 5F8-3A3 | EVQLVESGGGLVQPGGSLRLSCAASGFDFSDTHMSWVRQAPGKERELVAIITGGGSTD | 39 |
| | YVDSVKGRFTISRDNSKNTLYLQMNSLRAEDTAVYYCLARRTTIYWGQGTQVTVSS | |
| 5F8-8E3 | EVQLVESGGGLVQPGGSLRLSCAASGFDFSDTHMSWVRQAPGKERELVAIITSGGSTD | 40 |
| | YVDSVKGRFTISRDNSKNTLYLQMNSLRAEDTAVYYCNARLTTIYWGQGTQVTVSS | |
| 5F8-8D4 | EVQLVESGGGLVQPGGSLRLSCAASGFDFSDTFMSWVRQAPGKERELVAIITSGGSTD | 41 |
| | YVDSVKGRFTISRDNSKNTLYLQMNSLRAEDTAVYYCNARRTTIYWGQGTQVTVSS | |
| 5F8-4B12 | EVQLVESGGGLVQPGGSLRLSCAASGFDFPDTHMSWVRQAPGKERELVAIITSGGFTD | 42 |
| | YVDSVKGRFTISRDNSKNTLYLQMNSLRAEDTAVYYCRARRTTIYWGQGTQVTVSS | |
| 5F8-6D6 | EVQLVESGGGLVQPGGSLRLSCAASGFDFSDTAMSWVRQAPGKERELVAIITSGGSVD | 43 |
| | YVDSVKGRFTISRDNSKNTLYLQMNSLRAEDTAVYYCRARRTTIYWGQGTQVTVSS | |
| 5F8-8H3 | EVQLVESGGGLVQPGGSLRLSCAASGFDFSMTHMSWVRQAPGKERELVAIITSGGKT | 44 |
| | DYVDSVKGRFTISRDNSKNTLYLQMNSLRAEDTAVYYCNARRATIYWGQGTQVTVS | |
| | S | |
| 5F8-7D5 | EVQLVESGGGLVQPGGSLRLSCAASGFDFEDTHMSWVRQAPGKERELVAIITSGCSTD | 45 |
| | YVDSVKGRFTISRDNSKNTLYLQMNSLRAEDTAVYYCNARRATIYWGQGTQVTVSS | |

### Example 4: Characterization & Selection of Lead Human PD-L1 Functional Binders, and Hotspot Correction and Humanization of Top Clones

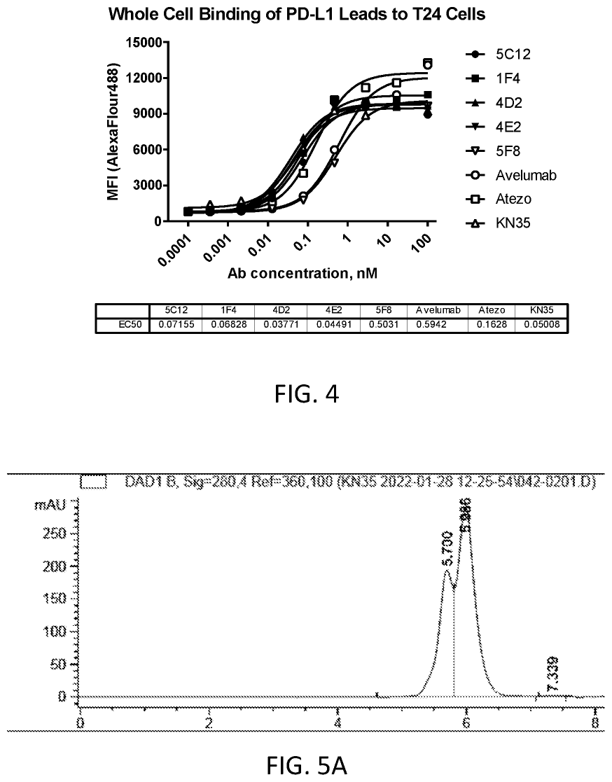

Binding abilities of lead human PD-L1 binders to human PD-L1 expressed on human tumor cells were determined using T24 cells, a human urinary bladder carcinoma cells which express endogenous human PD-L1 at a moderate level. Surprisingly, the high affinity clone in kinetic assay 5F8 has much lower binding ability to endogenously expressed human PD-L1 in T24 cells than clones of 5C12, 1F4, 4D2 and 4E2, a group of clones with the same CDR3 (FIG. 4 and table below). Therefore, we focused on this group for further investigation.

TABLE 10
EC50 of top h-PD-L1 VHH-Fc clones in WCB to
h-PD-L1 endogenously expressed in T24 cells

| PD-L1 lead clones | EC50, nM in WCB to T24 |
|---|---|
| 5F8 | 0.503 |
| 5C12 | 0.078 |
| 1F4 | 0.059 |
| 4D2 | 0.041 |
| 4E2 | 0.050 |
| KN35-hv | 0.054 |
| Avelumab | 0.336 |
| Atezo | 0.091 |

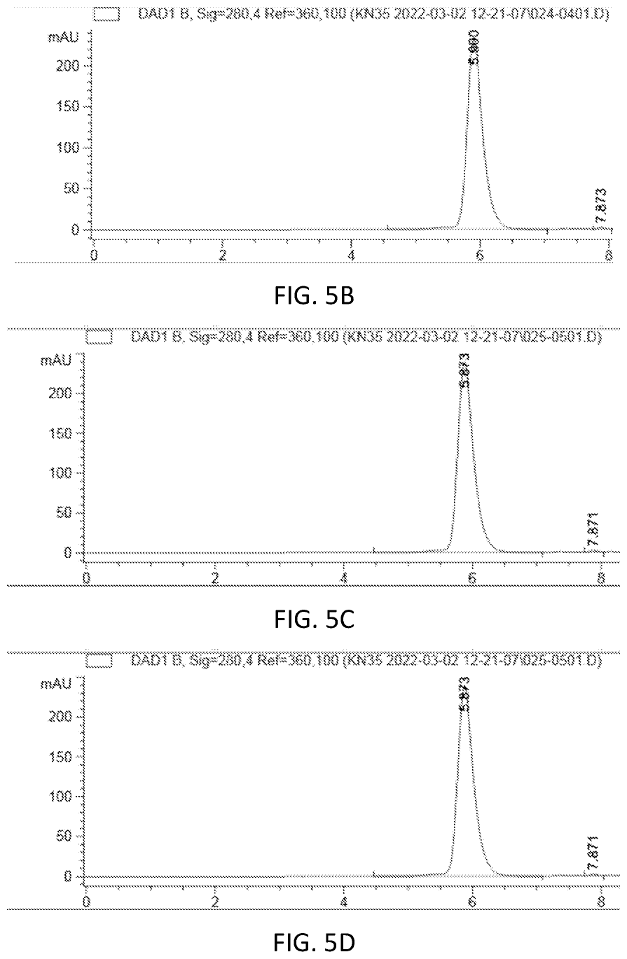

The CDR3 sequence of these 4 PD-L1 binders contains a hotspot (NGS), which induces N-glycosylation and changes the monomer ratio of proteins as shown in SEC-HPLC analytic assay (FIG. 5). To correct the hotspot in CDR3, three different designs were made. N was changed to T, S or Q. Sequences are shown in FIGS. 28 and 30, and the table below.

TABLE 11
CDR3 of hotspot corrected 5C12 variants (No change
of other CDRs and FRs for variants 46-48)

| Clone name | CDR3 | SEQ ID |
|---|---|---|
| 5C12 parental | NALVWNGSSYNN | 9 |
| 5C12-NQ | NALVWQGSSYNN | 46 |
| 5C12-NT | NALVWTGSSYNN | 47 |
| 5C12-NS | NALVWSGSSYNN | 48 |

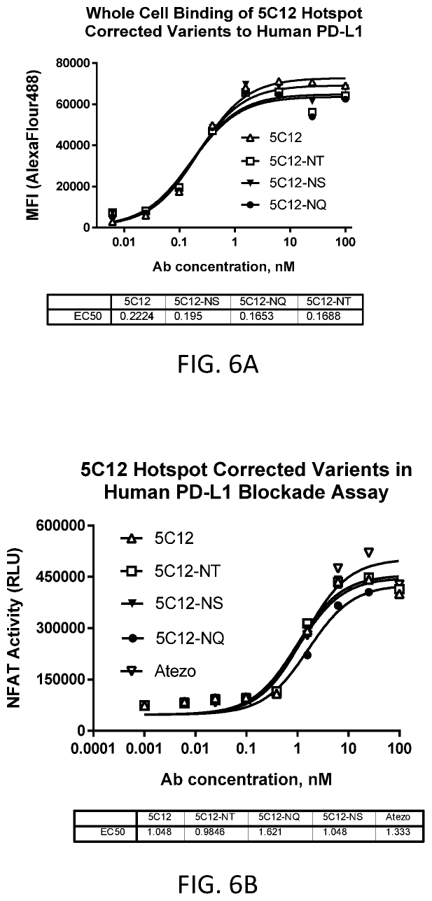

New variants with hotspot correction were analyzed with SEC-HPLC and examined in whole cell binding assays and PD-L1 blockade reporter assays. Changing N to either T, S or Q corrected the N-glycosylation and SEC-HPLC demonstrated much higher monomer ratios (FIG. 5). The whole cell binding ability and potency in blockade reporter assays were retained as compared to their parental clone as shown (FIGS. 6A & 6B).

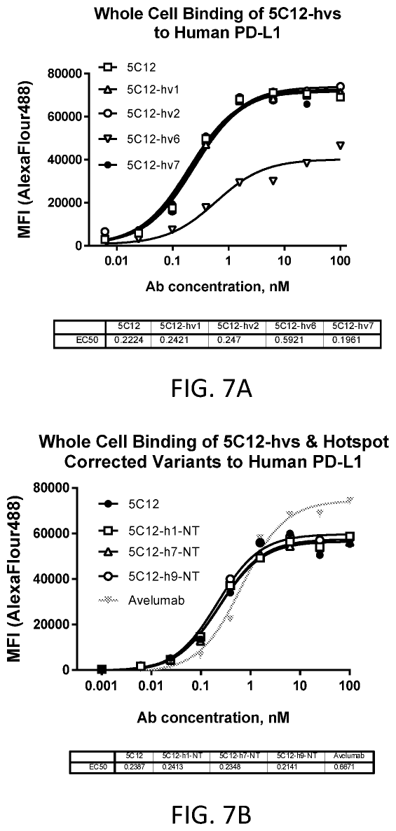

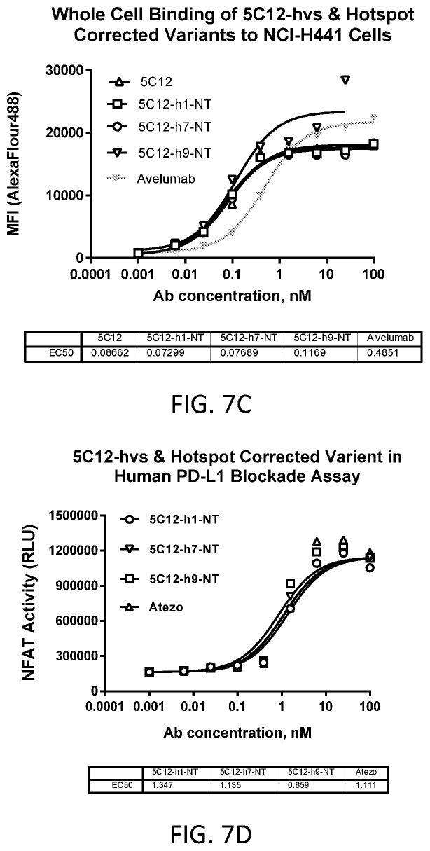

Humanization of 5C12 clone was performed by analysis of germline sequence using Igblast. One of the closest human germline of 5C12 is IGHV-3-11.01. The framework of Alpaca sequence of 5C12 was changed to that of IGHV-3-11.01 with the exception of most amino acid sequence in framework 2 (particularly the three hallmark of Alpaca/IIama sequences of QREF at 44, 45, 47), which were preserved due to the impact of these amino acids in stability and affinity. 9 different humanized variants were made with slightly different designs in humanization of framework 2 and 3. The humanized VHHs were fused with human IgG1 Fc and proteins were produced by transient transfection to ExpiCHO cells and purified by Protein A resin. Whole cell binding assays were performed using h-PD-L1 stably expressing cells. Representative variants with high binding abilities to h-PD-L1 were shown in FIG. 7A. hv1, hv2 and hv7 showed similar binding ability as parental clone 5C12 (EC50 at 0.22 nM). Hotspot corrected humanized variants were also made and the whole cell binding to CHO cells stably expressing h-PD-L1 & human tumor cells NCI-H441 endogenously expressing h-PD-L1 were determined (FIGS. 7B and 7C). PD-L1 blockade function was determined by reporter assay (FIG. 7D). Results indicated that the 5C12-hv9-NT variant has the highest binding ability and potency. The sequences of 5C12 humanized and CDR3 hotspot corrected variants are shown in FIGS. 28 and 30, and the table below.

TABLE 12
Sequences of 5C12 CDR3 hotspot corrected & humanized variants

| Clone names | FR1CDR1FR2CDR2FR3CDR3FR4 | SEQ ID |
|---|---|---|
| 5C12-Alpaca | QVQLVESGGGLVQAGGSLRLSCAASGFSASDVVMAWYRQAPGKQREFVA | 9 |
| | NIAARGSTDYANSVKGRFTISRDNAKNTVYLQMNSLKSDDTAVYYCNAL | |
| | VWNGSSYNNWGQGTQVTVSS | |
| 5C12-hv1 | QVQLVESGGGLVKPGGSLRLSCAASGFSASDVVMAWYRQAPGKQREFVA | 49 |
| | NIAARGSTDYADSVKGRFTISRDNAKNSLYLQMNSLRAEDTAVYYCNAL | |
| | VWNGSSYNNWGQGTQVTVSS | |
| 5C12-hv1-NQ | QVQLVESGGGLVKPGGSLRLSCAASGFSASDVVMAWYRQAPGKQREFVA | 50 |
| | NIAARGSTDYADSVKGRFTISRDNAKNSLYLQMNSLRAEDTAVYYCNAL | |
| | VWQGSSYNNWGQGTQVTVSS | |
| 5C12-hv1-NT | QVQLVESGGGLVKPGGSLRLSCAASGFSASDVVMAWYRQAPGKQREFVA | 51 |
| | NIAARGSTDYADSVKGRFTISRDNAKNSLYLQMNSLRAEDTAVYYCNAL | |
| | VWTGSSYNNWGQGTQVTVSS | |
| 5C12-hv1-NS | QVQLVESGGGLVKPGGSLRLSCAASGFSASDVVMAWYRQAPGKQREFVA | 52 |
| | NIAARGSTDYADSVKGRFTISRDNAKNSLYLQMNSLRAEDTAVYYCNAL | |
| | VWSGSSYNNWGQGTQVTVSS | |
| 5C12-hv2 | QVQLVESGGGLVKPGGSLRLSCAASGFSASDVVMSWYRQAPGKQREFVA | 53 |
| | NIAARGSTDYADSVKGRFTISRDNAKNSLYLQMNSLRAEDTAVYYCNAL | |
| | VWNGSSYNNWGQGTQVTVSS | |
| 5C12-hv3 | QVQLVESGGGLVKPGGSLRLSCAASGFSASDVVMSWYRQAPGKQREFVA | 54 |
| | YIAARGSTDYADSVKGRFTISRDNAKNSLYLQMNSLRAEDTAVYYCNAL | |
| | VWNGSSYNNWGQGTQVTVSS | |
| 5C12-hv4 | QVQLVESGGGLVKPGGSLRLSCAASGFSASDVVMSWYRQAPGKQREFVS | 55 |
| | YIAARGSTDYADSVKGRFTISRDNAKNSLYLQMNSLRAEDTAVYYCNAL | |
| | VWNGSSYNNWGQGTQVTVSS | |
| 5C12-hv5 | QVQLVESGGGLVKPGGSLRLSCAASGFSASDVVMSWYRQAPGKQREFVS | 56 |
| | YIAARGSTDYANSVKGRFTISRDNAKNSLYLQMNSLRAEDTAVYYCNAL | |
| | VWNGSSYNNWGQGTQVTVSS | |
| 5C12-hv6 | QVQLVESGGGLVKPGGSLRLSCAASGFSASDVVMSWYRQAPGKQREFVS | 57 |
| | YIAARGSTYYADSVKGRFTISRDNAKNSLYLQMNSLRAEDTAVYYCNAL | |
| | VWNGSSYNNWGQGTQVTVSS | |
| 5C12-hv7 | QVQLVESGGGLVKPGGSLRLSCAASGFSASDVVMAWYRQAPGKQREFVS | 58 |
| | NIAARGSTDYADSVKGRFTISRDNAKNSLYLQMNSLRAEDTAVYYCNAL | |
| | VWNGSSYNNWGQGTQVTVSS | |
| 5C12-hv8 | QVQLVESGGGLVKPGGSLRLSCAASGFSASDVVMAWYRQAPGKQREFVS | 59 |
| | YIAARGSTDYADSVKGRFTISRDNAKNSLYLQMNSLRAEDTAVYYCNAL | |
| | VWNGSSYNNWGQGTQVTVSS | |
| 5C12-hv7-NT | QVQLVESGGGLVKPGGSLRLSCAASGFSASDVVMAWYRQAPGKQREFVS | 60 |
| | NIAARGSTDYADSVKGRFTISRDNAKNSLYLQMNSLRAEDTAVYYCNAL | |
| | VWTGSSYNNWGQGTQVTVSS | |
| 5C12-hv9-NT | QVQLVESGGGLVKPGGSLRLSCAASGFSASDVVMAWYRQAPGKQREFVS | 61 |
| | NIAARGSTYYADSVKGRFTISRDNAKNSLYLQMNSLRAEDTAVYYCNAL | |
| | VWTGSSYNNWGQGTQVTVSS | |

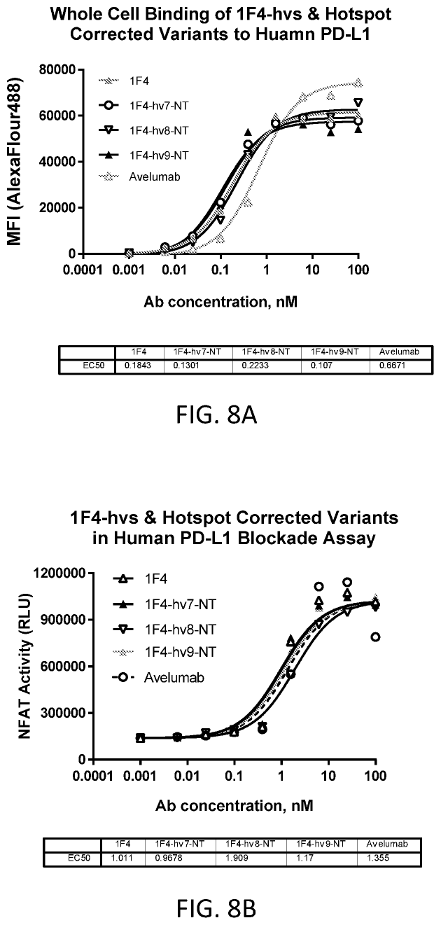

Humanization of 1F4 was performed with the same strategy as that for clone 5C12 using human germline IGHV-3-11.01 or IGHV-3-23.04. Twelve Humanized variants were made (all fused with human IgG1 Fc) and their CDR3 hotspot was corrected (N to T). Their binding abilities to h-PD-L1 were examined by whole cell binding assays. Results of clones with high binding abilities are shown in FIG. 8A and their potencies in blocking PD-L1 were tested in PD-L1 blockade reporter assay, and results are shown in FIG. 8B. Sequences of humanized 1F4 clones shown in FIGS. 28 and 30, and the table below. 1F4-hv7 and hv9 retained high binding ability and potency compared to parental clone 1F4.

TABLE 13
Sequences of 1F4 CDR3 hotspot corrected & humanized variants

| Clone names | FR1CDR1FR2CDR2FR3CDR3FR4 | SEQ ID |
|---|---|---|
| 1F4-Alpaca | QVQLVESGGGLVQAGGSLRLSCAASGFSASDVVMAWYRQAPGKQREFVAN | 10 |
| | IAASSTTRYADSVKGRFTISRDDAKNTVSLQMNSLKSEDTAVYYCNALVW | |
| | NGSSYNNWGQGTQVTVSS | |
| 1F4-hv1-NT | EVQLVESGGGLVQPGGSLRLSCAASGFSASDVVMSWVRQAPGKQREFVSA | 62 |
| | IAASSTTYYADSVKGRFTISRDNSKNTLYLQMNSLRAEDTAVYYCNALVW | |
| | TGSSYNNWGQGTQVTVSS | |
| 1F4-hv2-NT | EVQLVESGGGLVQPGGSLRLSCAASGFSASDVVMSWVRQAPGKQREFVAA | 63 |
| | IAASSTTYYADSVKGRFTISRDNSKNTLYLQMNSLRAEDTAVYYCNALVW | |
| | TGSSYNNWGQGTQVTVSS | |
| 1F4-hv3-NT | QVQLVESGGGLVKPGGSLRLSCAASGFSASDVVMSWIRQAPGKQREFVSY | 64 |
| | IAASSTTYYADSVKGRFTISRDNAKNSLYLQMNSLRAEDTAVYYCNALVW | |
| | TGSSYNNWGQGTQVTVSS | |
| 1F4-hv4-NT | QVQLVESGGGLVQPGGSLRLSCAASGFSASDVVMSWIRQAPGKQREFVSA | 65 |
| | IAASSTTYYADSVKGRFTISRDNAKNSLYLQMNSLRAEDTAVYYCNALVW | |
| | TGSSYNNWGQGTQVTVSS | |
| 1F4-hv5-NT | QVQLVESGGGLVKPGGSLRLSCAASGFSASDVVMSWYRQAPGKQREFVAY | 66 |
| | IAASSTTRYADSVKGRFTISRDNAKNSLYLQMNSLRAEDTAVYYCNALVW | |
| | TGSSYNNWGQGTQVTVSS | |
| 1F4-hv6-NT | QVQLVESGGGLVKPGGSLRLSCAASGFSASDVVMSWYRQAPGKQREFVSY | 67 |
| | IAASSTTRYADSVKGRFTISRDNAKNSLYLQMNSLRAEDTAVYYCNALVW | |
| | TGSSYNNWGQGTQVTVSS | |
| 1F4-hv7-NT | QVQLVESGGGLVKPGGSLRLSCAASGFSASDVVMAWYRQAPGKQREFVAN | 68 |
| | IAASSTTRYADSVKGRFTISRDNAKNSLYLQMNSLRAEDTAVYYCNALVW | |
| | TGSSYNNWGQGTQVTVSS | |
| 1F4-hv8-NT | QVQLVESGGGLVKPGGSLRLSCAASGFSASDVVMAWYRQAPGKQREFVAN | 69 |
| | IAASSTTYYADSVKGRFTISRDNAKNSLYLQMNSLRAEDTAVYYCNALVW | |
| | TGSSYNNWGQGTQVTVSS | |
| 1F4-hv9-NT | QVQLVESGGGLVKPGGSLRLSCAASGFSASDVVMAWYRQAPGKQREFVSN | 70 |
| | IAASSTTRYADSVKGRFTISRDNAKNSLYLQMNSLRAEDTAVYYCNALVW | |
| | TGSSYNNWGQGTQVTVSS | |
| 1F4-hv10-NT | QVQLVESGGGLVKPGGSLRLSCAASGFSASDVVMAWYRQAPGKQREFVSN | 71 |
| | IAASSTTYYADSVKGRFTISRDNAKNSLYLQMNSLRAEDTAVYYCNALVW | |
| | TGSSYNNWGQGTQVTVSS | |
| 1F4-hv11-NT | QVQLVESGGGLVKPGGSLRLSCAASGFSASDVVMAWYRQAPGKQREFVSY | 72 |
| | IAASSTTYYADSVKGRFTISRDNAKNSLYLQMNSLRAEDTAVYYCNALVW | |
| | TGSSYNNWGQGTQVTVSS | |
| 1F4-hv12-NT | QVQLVESGGGLVKPGGSLRLSCAASGFSASDVVMAWYRQAPGKQREFVSY | 73 |
| | IAASSTTRYADSVKGRFTISRDNAKNSLYLQMNSLRAEDTAVYYCNALVW | |
| | TGSSYNNWGQGTQVTVSS | |

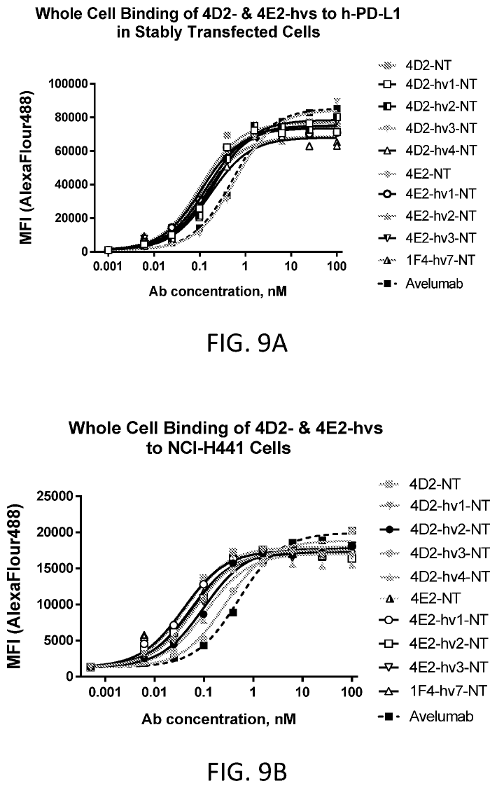

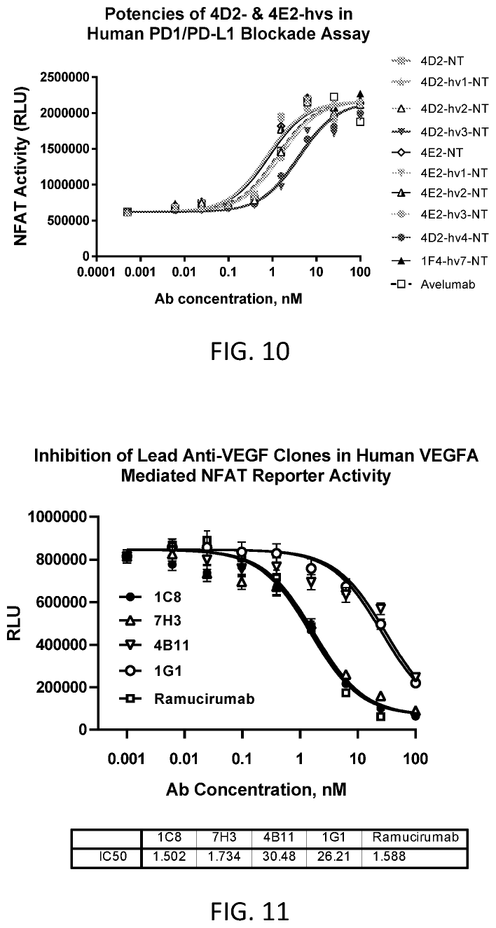

Humanization and CDR3 hotspot correction of clone 4D2 and 4E2 were carried out following the same strategy for 5C12. The whole cell binding of humanized variants (all fused with human IgG1 Fc) to CHO cells stably expressing h-PD-L1 and human tumor cells NCI-H441 endogenously expressing h-PD-L1 are shown in FIGS. 9A & 9B. The potency in blocking human PD-L1 was determined, and the results are shown in FIG. 10. Humanized variants with CDR3 hotspot correction can retain the binding activity to human PD-L1 and potency in blocking human PD-L1, with high binding ability and potency observed among 4D2-hv1-NT, 4E2-hv1-NT and 4E2-hv2-NT (FIGS. 9-10 and the table below).

TABLE 14
WCB to PD-L1 and potency in blocking PD-L1
for 4D2 & 4E2 humanized variants

| | EC50, nM in | | |
|---|---|---|---|
| | WCB to CHO-h- | EC50, nM in WCB to | EC50, nM in hPD-L1 |
| Clone names | PD-L1 cells | NCI-H441 cells | Blockade Assay |
| 4D2-Alpaca-NT | 0.103 | 0.044 | 0.729 |
| 4D2-hv1-NT | 0.143 | 0.065 | 0.915 |
| 4D2-hv2-NT | 0.203 | 0.099 | 1.323 |
| 4D2-hv3-NT | 0.598 | 0.271 | 4.177 |
| 4D2-hv4-NT | 0.171 | 0.112 | 3.779 |
| 4E2-Alpaca-NT | 0.109 | 0.041 | 0.812 |
| 4E2-hv1-NT | 0.110 | 0.039 | 1.128 |
| 4E2-hv2-NT | 0.110 | 0.054 | 1.083 |
| 4E2-hv3-NT | 0.167 | 0.055 | 1.556 |
| 1F4-hv7-NT | 0.130 | 0.056 | 0.960 |
| Avelumab | 0.526 | 0.501 | 1.55 |

The sequences of parental Alpaca clones and humanized variants shown in FIGS. 28 and 30, and the table below.

TABLE 15
Sequences of 4D2 and 4E2 humanized variants

| Clone names | FR1CDR1FR2CDR2FR3CDR3FR4 | SEQ ID |
|---|---|---|
| 4D2_Alpaca- | QVQLVESGGGLVQAGGSLRLSCAASGFSASDVVMAWYRQAPGKQREFVANLGAS | 200 |
| NT | STTRYADSVKGRFTISRDDAKNTVSLQMNSLKSEDTAVYYCNALVWTGSSYNNW | |
| | GQGTQVTVSS | |
| 4D2-hv1-NT | QVQLVESGGGLVKPGGSLRLSCAASGFSASDVVMAWYRQAPGKQREFVANLGAS | 74 |
| | STTRYADSVKGRFTISRDNAKNSLYLQMNSLRAEDTAVYYCNALVWTGSSYNNW | |
| | GQGTQVTVSS | |
| 4D2-hv2-NT | QVQLVESGGGLVKPGGSLRLSCAASGFSASDVVMAWYRQAPGKQREFVSNLGAS | 75 |
| | STTRYADSVKGRFTISRDNAKNSLYLQMNSLRAEDTAVYYCNALVWTGSSYNNW | |
| | GQGTQVTVSS | |
| 4D2-hv3-NT | QVQLVESGGGLVKPGGSLRLSCAASGFSASDVVMAWYRQAPGKQREFVSNLGAS | 76 |
| | STTYYADSVKGRFTISRDNAKNSLYLQMNSLRAEDTAVYYCNALVWTGSSYNNW | |
| | GQGTQVTVSS | |
| 4D2-hv4-NT | QVQLVESGGGLVKPGGSLRLSCAASGFSASDVVMAWYRQAPGKQREFVSNLGAS | 77 |
| | STTNYADSVKGRFTISRDNAKNSLYLQMNSLRAEDTAVYYCNALVWTGSSYNNW | |
| | GQGTQVTVSS | |
| 4E2_Alpaca- | EVQLVESGGGSVQDGGSLTLSCAASGFSAGDVVMAWYRQAPGKQREFVANIAAS | 201 |
| NT | STTRYTDSVKGRFTISRDNAKNTVYLQMNSLKSEDTAVYYCNALVWTGSSYNNW | |
| | GQGTQVTVSS | |
| 4E2-hv1-NT | EVQLVESGGGLVKPGGSLRLSCAASGFSAGDVVMAWYRQAPGKQREFVANIAAS | 78 |
| | STTRYADSVKGRFTISRDNAKNSLYLQMNSLRAEDTAVYYCNALVWTGSSYNNW | |
| | GQGTQVTVSS | |
| 4E2-hv2-NT | EVQLVESGGGLVKPGGSLRLSCAASGFSAGDVVMAWYRQAPGKQREFVSNIAAS | 79 |
| | STTRYADSVKGRFTISRDNAKNSLYLQMNSLRAEDTAVYYCNALVWTGSSYNNW | |
| | GQGTQVTVSS | |
| 4E2-hv3-NT | EVQLVESGGGLVKPGGSLRLSCAASGFSAGDVVMAWYRQAPGKQREFVSNIAAS | 80 |
| | STTYYADSVKGRFTISRDNAKNSLYLQMNSLRAEDTAVYYCNALVWTGSSYNNW | |
| | GQGTQVTVSS | |

Kinetic binding affinities of selected lead humanized clones (all fused with human IgG1 Fc) were determined using BLI. All lead clones showed good association curve with human PD-L1. The affinities calculated by Koff and Kon are shown in the table below for human PD-L1.

TABLE 16
Binding affinities of lead PD-L1 clones to h-PD-L1

| Loading | koff(1/s) | kon(1/Ms) | KD(M) |
|---|---|---|---|
| 4D2-hv1-NT | 1.74E−03 | 3.84E+05 | 4.54E−09 |
| 4E2-hv1-NT | 2.25E−03 | 4.85E+05 | 4.64E−09 |
| 4E2-hv2-NT | 2.48E−03 | 5.23E+05 | 4.75E−09 |
| 1F4-hv7-NT | 3.41E−03 | 5.33E+05 | 6.39E−09 |
| 5C12-hv7-NT | 4.37E−03 | 5.39E+05 | 8.12E−09 |

The affinities calculated by Koff and Kon are shown in the table below for cynomolgus PD-L1 (cyno-PD-L1).

TABLE 17
Binding affinities of lead PD-L1 clones to cyno-PD-L1

| Loading | koff(1/s) | kon(1/Ms) | KD(M) |
|---|---|---|---|
| 4D2-hv1-NT | 2.06E−03 | 5.47E+05 | 3.76E−09 |
| 4E2-hv1-NT | 2.10E−03 | 6.18E+05 | 3.40E−09 |
| 4E2-hv2-NT | 2.10E−03 | 6.47E+05 | 3.24E−09 |
| 1F4-hv7-NT | 2.34E−03 | 6.05E+05 | 3.87E−09 |
| 5C12-hv7-NT | 2.57E−03 | 5.92E+05 | 4.34E−09 |

### Example 5: Synthetic VHH Antibody Library Construction

Camelid/Alpaca VHH antibody is a type of heavy chain only antibody, which preserves binding (and function) to antigens similar to conventional antibodies containing both heavy and light chains. The unique feature of VHH is that it has a longer CDR3 compared to the CDR3 of the conventional antibody heavy chain. Using “in silicon” design to make synthetic VHH library has become more common recently. Based on published information, we designed our own VHH synthetic library for binders of VEGF. We used fixed length for CDR1 & 2, each containing 8 amino acids. For CDR3, we designed 13 different lengths from 10 to 22 amino acids and in a different ratio. The genes of the designed VHH library were synthesized and cloned into pADL-23c phagemids. The phagemid VHH library was used to transform TG1 cells. 1.5 μg phagemid DNA produced >2.5×10⁹ TG1 colonies. 12 transformations were made, and the combined TG1 VHH library was about 3×10¹⁰. Phage library was made by culturing 2L of TG1 cells and 20 times helper phages and purified following a standard protocol. The final titer of phage library (measured at OD260) was 2.37×10¹³/mL.

### Example 6: Panning of Anti-VEGFA VHH Antibodies

Antigen of recombinant human/cynomolgus VEGF165 (an isoform of h-VEGFA) was purchased from Sinobiological USA, and biotinylated with NHS-ester biotinylation reagent (EZ-Link Sulfo-NHS-SS-Biotin, ThermoFisher). Panning of binders to human/cynomolgus VEGF165 antigen was performed using streptavidin-coupled Dynabeads coated with biotinylated VEGF165. After 3 rounds of panning, binders of VEGF165 were eluted and infected to SS320 cells. Colonies of SS320 cells were picked and cultured in 2YT medium and IPTG was added for secretion of VHH antibodies. Supernatants with VHH antibodies were screened by ELISA assays using VEGF165 coated high-binding plates. 9 plates (about 864 samples) were screened, and 176 ELISA positive binders obtained. Inhibition of VHH supernatants on VEGF165 mediated reporter activity was measured, and cross-binding to mouse VEGF164 (an isoform of mouse VEGFA) was determined. Both human and mouse VEGFA blockers were selected (total 72 clones) for sequencing and 42 sequence unique clones were obtained. Cross-binding of sequence unique clones to subtypes of VEGF B, C, D, & PIGF was examined, and cross-binders were eliminated. 19 unique clones were selected to make bivalent IgG1 or IgG4 Fc clones.

### Example 7: Affinity and Potency of Bivalent Anti-VEGF VHH-Fc Clones

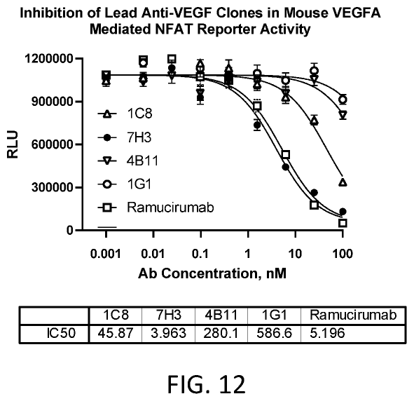

Anti-VEGFA VHH clones were fused with human IgG4 Fc (hinge+CH2+CH3), cloned into pcDNA3.4 vectors, and transfected to CHO cells for protein production. VHH-Fc proteins were purified by Protein A resin. Blockade function of VHH-Fc clones to human VEGFA was determined using a luciferase reporter assay, with a stable cell line overexpressing VEGFA receptor KDR and an NFAT-luciferase reporter gene. Results of top 4 clones in blocking human VEGFA are shown in FIG. 11. Clone 7H3 and 1C8 have similar potency to reference antibody Ramucirumab. Results of these 4 clones in blocking mouse VEGFA (FIG. 12) showed that 7H3 was more potent than Ramucirumab. The binding affinities of these top 4 VHH-Fc clones to human VEGFA were also determined using BLI kinetic assays (see table below). 7H3 showed the highest affinity to human VEGFA.

TABLE 18
Binding affinities to human VEGFA

| Clones | koff(1/s) | kon(1/Ms) | KD(M) |
|---|---|---|---|
| 7H3 | 2.71E−04 | 1.30E+05 | 2.08E−09 |
| 1C8 | 1.77E−04 | 6.80E+04 | 2.61E−09 |
| 4B11 | 6.52E−04 | 2.31E+05 | 2.82E−09 |
| 1G1 | 2.62E−03 | 1.82E+05 | 1.44E−08 |

The sequences of these 4 clones shown FIGS. 29 and 30, and the table below.

TABLE 19
Sequences of top 4 anti-VEGF clones

| Clone | FR1CDR1FR2CDR2FR3CDR3FR4 | SEQ ID |
|---|---|---|
| 7H3 | QVQLVESGGGVVQPGRSLRLSCAASGFTFGPYAMHWVRQAPGKERELVAAIWPSGSIT | 81 |
| | YYADSVKGRFTISRDNSKNTLYLQMNSLRAEDTAVYYCAFGSPTGSITYWGQGTLVTV | |
| | SS | |
| 1C8 | QVQLVESGGGVVQPGRSLRLSCAASGRSFSRYAMAWVRQAPGKERESVAVIGSGGGRT | 82 |
| | YYADSVKGRFTISRDNSKNTLYLQMNSLRAEDTAVYYCAARIYRVVGSGTYPAGYDYW | |
| | GQGTLVTVSS | |
| 4B11 | QVQLVESGGGVVQPGRSLRLSCAASGLSFSQYEMHWVRQAPGKEREFVAAISSSGIST | 83 |
| | YYADSVKGRFTISRDNSKNTLYLQMNSLRAEDTAVYYCAAELVDYHRWGYDYWGQGTL | |
| | VTVSS | |
| 1G1 | QVQLVESGGGVVQPGRSLRLSCAASGLSFSYGTMAWVRQAPGKELELVAALSAGGTIT | 84 |
| | YYADSVKGRFTISRDNSKNTLYLQMNSLRAEDTAVYYCAALGGYSGGSKYPYYDYWGQ | |
| | GTLVTVSS | |

### Example 8: Construction of Bispecific Anti-PD-L1× Anti-VEGF Antibodies and Potencies of Bispecific Antibodies in Blocking PD-L1 and VEGFA

Lead humanized anti-human PD-L1 clones were used to make bispecific antibodies with lead anti-VEGF clone 7H3. First, 4 different formats of BsAbs were made using 4E2-hv2-NT clone for anti-PD-L1 arm and 7H3 for anti-VEGFA arm. IgG1 hinge and CH2+CH3 were used for all these constructions. The diagram shown in FIGS. 13A-13D and sequences shown in the four tables below.

TABLE 20
Sequence of 4E2_Fc_7H3 (SEQ ID NO: 85)

| | | Linker2 | 7H3 |
|---|---|---|---|
| 4E2-hv2-NT | Hinge+CH2+CH3 (IgG1) | (SEQ ID | (SEQ ID |
| (SEQ ID NO: 79) | (SEQ ID NO: 204) | NO: 203) | NO: 81) |
| EVQLVESGGGLVKPGGSL | EPKSSDKTHTCPPCPAPELLGGPSVFL | GSGGSGGSGG | QVQLVESGGGVVQP |
| RLSCAASGFSAGDVVMAW | FPPKPKDTLMISRTPEVTCVVVDVSHE | SGGSG | GRSLRLSCAASGFT |
| YRQAPGKQREFVSNIAAS | DPEVKFNWYVDGVEVHNAKTKPREEQY | | FGPYAMHWVRQAPG |
| STTRYADSVKGRFTISRD | NSTYRVVSVLTVLHQDWLNGKEYKCKV | | KERELVAAIWPSGS |
| NAKNSLYLQMNSLRAEDT | SNKALPAPIEKTISKAKGQPREPQVYT | | ITYYADSVKGRFTI |
| AVYYCNALVWTGSSYNNW | LPPSREEMTKNQVSLTCLVKGFYPSDI | | SRDNSKNTLYLQMN |
| GQGTQVTVSS | AVEWESNGQPENNYKTTPPVLDSDGSF | | SLRAEDTAVYYCAF |
| | FLYSKLTVDKSRWQQGNVFSCSVMHEA | | GSPTGSITYWGQGT |
| | LHNHYTQKSLSLSPGK | | LVTVSS |

TABLE 21
Sequence of 4E2+7H3_Fc (SEQ ID NO: 86)

| | Linnker1 | | |
|---|---|---|---|
| 4E2-hv2-NT | (SEQ ID | 7H3 | Hinge+CH2+CH3 (IgG1) |
| (SEQ ID NO: 79) | NO: 202) | (SEQ ID NO: 81) | (SEQ ID NO: 204) |
| EVQLVESGGGLVKPGG | GSGGSG | QVQLVESGGGVVQPG | EPKSSDKTHTCPPCPAPELLGGPSVFLFPPKP |
| SLRLSCAASGFSAGDV | GSGGSG | RSLRLSCAASGFTFG | KDTLMISRTPEVTCVVVDVSHEDPEVKFNWYV |
| VMAWYRQAPGKQREFV | | PYAMHWVRQAPGKER | DGVEVHNAKTKPREEQYNSTYRVVSVLTVLHQ |
| SNIAASSTTRYADSVK | | ELVAAIWPSGSITYY | DWLNGKEYKCKVSNKALPAPIEKTISKAKGQP |
| GRFTISRDNAKNSLYL | | ADSVKGRFTISRDNS | REPQVYTLPPSREEMTKNQVSLTCLVKGFYPS |
| QMNSLRAEDTAVYYCN | | KNTLYLQMNSLRAED | DIAVEWESNGQPENNYKTTPPVLDSDGSFFLY |
| ALVWTGSSYNNWGQGT | | TAVYYCAFGSPTGSI | SKLTVDKSRWQQGNVFSCSVMHEALHNHYTQK |
| QVTVSS | | TYWGQGTLVTVSS | SLSLSPGK |

TABLE 22
sequence of 7H3+4E2_Fc (SEQ ID NO: 87)

| 7H3 | Linker1 | 4E2-hv2-NT | |
|---|---|---|---|
| (SEQ ID | (SEQ ID | (SEQ ID | Hinge+CH2+CH3 (IgG1) |
| NO: 81) | NO: 202) | NO: 79) | (SEQ ID NO: 204) |
| QVQLVESGGGVVQP | GSGGS | EVQLVESGGGLVKP | EPKSSDKTHTCPPCPAPELLGGPSVFLFPPKPKDT |
| GRSLRLSCAASGFT | GGSGG | GGSLRLSCAASGFS | LMISRTPEVTCVVVDVSHEDPEVKFNWYVDGVEVH |
| FGPYAMHWVRQAPG | SG | AGDVVMAWYRQAPG | NAKTKPREEQYNSTYRVVSVLTVLHQDWLNGKEYK |
| KERELVAAIWPSGS | | KQREFVSNIAASST | CKVSNKALPAPIEKTISKAKGQPREPQVYTLPPSR |
| ITYYADSVKGRFTI | | TRYADSVKGRFTIS | EEMTKNQVSLTCLVKGFYPSDIAVEWESNGQPENN |
| SRDNSKNTLYLQMN | | RDNAKNSLYLQMNS | YKTTPPVLDSDGSFFLYSKLTVDKSRWQQGNVFSC |
| SLRAEDTAVYYCAF | | LRAEDTAVYYCNAL | SVMHEALHNHYTQKSLSLSPGK |
| GSPTGSITYWGQGT | | VWTGSSYNNWGQGT | |
| LVTVSS | | QVTVSS | |

TABLE 23
sequence of 7H3_Fc_4E2 (SEQ ID NO: 88)

| | | Linker2 | 4E-hv2-NT |
|---|---|---|---|
| 7H3 | Hinge+CH2+CH3 (IgG1) | (SEQ ID | (SEQ ID |
| (SEQ ID NO: 81) | (SEQ ID NO: 204) | NO: 203) | NO: 79) |
| QVQLVESGGGVVQPGR | EPKSSDKTHTCPPCPAPELLGGPSVFLFP | GSGGSG | EVQLVESGGGLVK |
| SLRLSCAASGFTFGPY | PKPKDTLMISRTPEVTCVVVDVSHEDPEV | GSGGSG | PGGSLRLSCAASG |
| AMHWVRQAPGKERELV | KFNWYVDGVEVHNAKTKPREEQYNSTYRV | GSG | FSAGDVVMAWYRQ |
| AAIWPSGSITYYADSV | VSVLTVLHQDWLNGKEYKCKVSNKALPAP | | APGKQREFVSNIA |
| KGRFTISRDNSKNTLY | IEKTISKAKGQPREPQVYTLPPSREEMTK | | ASSTTRYADSVKG |
| LQMNSLRAEDTAVYYC | NQVSLTCLVKGFYPSDIAVEWESNGQPEN | | RFTISRDNAKNSL |
| AFGSPTGSITYWGQGT | NYKTTPPVLDSDGSFFLYSKLTVDKSRWQ | | YLQMNSLRAEDTA |
| LVTVSS | QGNVFSCSVMHEALHNHYTQKSLSLSPGK | | VYYCNALVWTGSS |
| | | | YNNWGQGTQVTVS |
| | | | S |

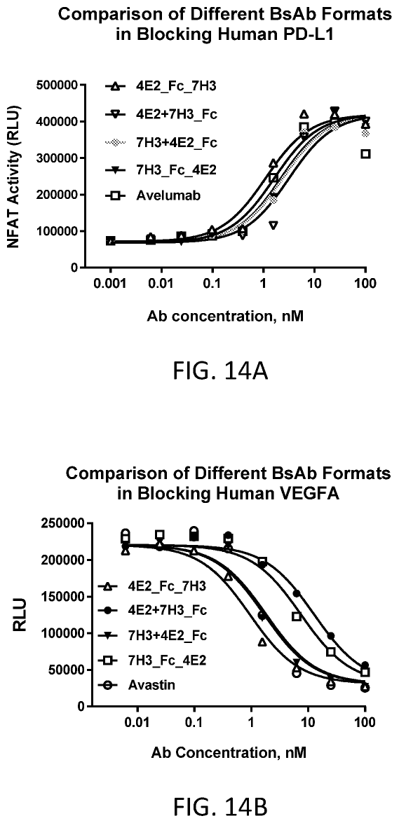

Potencies of their anti-PD-L1 arm in blocking human PD-L1 were tested, results are shown in FIG. 14A and the table below. The format of 4E2_Fc_7H3 showed the highest potency in blocking h-PD-L1 among these different formats of BsAbs.

Potencies of their anti-VEGFA arm were also tested, and results are similar to that of the anti-PD-L1 arm. The format of 4E2 Fc_7H3 showed the highest potency in blocking h-VEGFA as shown in FIG. 14B and the table below.

TABLE 24
Potencies of different BsAb formats in blocking
h-PD-L1 & h-VEGFA and their SEC-HPLC profile

| | | EC50 (nM) in | |
|---|---|---|---|
| | EC50 (nM) in | blocking h- | % monomer in |
| Clones | blocking h-PD-L1 | VEGFA | SEC |
| 4E2_Fc_7H3 | 1.11 | 1.01 | 98.0 |
| (SEQ ID NO: 85) | | | |
| 4E2 + 7H3_Fc | 3.14 | 13.75 | 79.0 |
| SEQ ID NO: 86) | | | |
| 7H3 + 4E2_Fc | 2.41 | 1.95 | 87.3 |
| (SEQ ID NO: 87) | | | |
| 7H3_Fc_4E2 | 2.18 | 8.02 | 82.2 |
| (SEQ ID NO: 88) | | | |
| Avelumab | 1.61 | No | 97.2 |
| Avastin | No | 1.79 | 97.1 |
| Ramucirumab | No | 4.05 | ND |

No = no activity.
ND = not determined

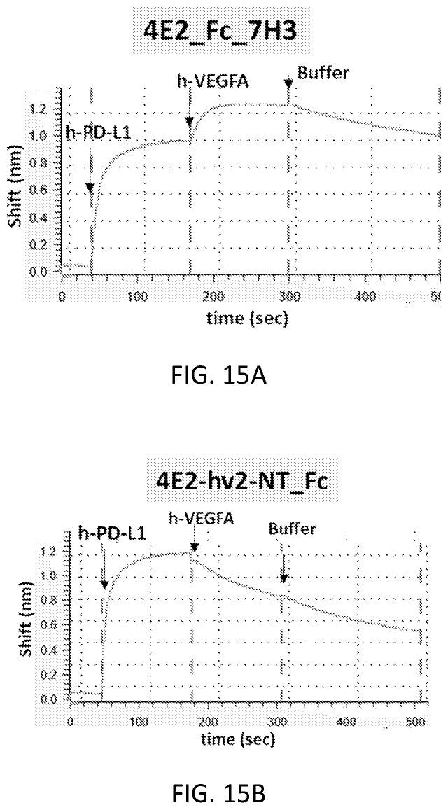

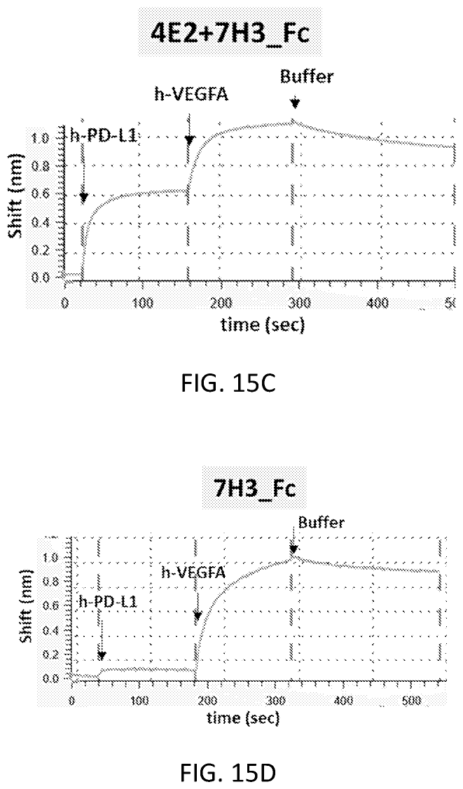

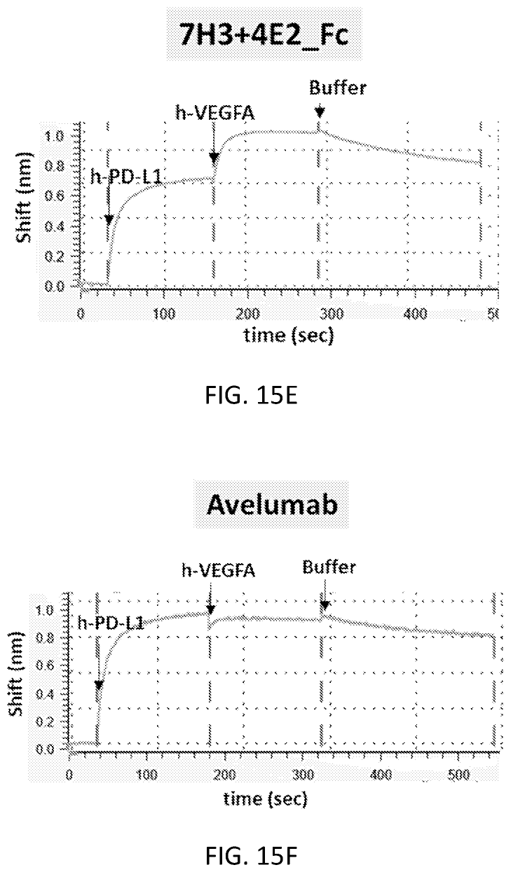

Simultaneous binding of these BsAbs to both h-PD-L1 and h-VEGFA antigens was tested using double binding assays (FIG. 15). All BsAbs are able to bind to both h-PD-L1 and h-VEGFA. By contrast, the parental monospecific antibody (4E2-hv2-NT_Fc or 7H3_Fc) only binds to either h-PD-L1 or h-VEGFA. Reference anti-PD-L1 antibody avelumab only binds to h-PD-L1.

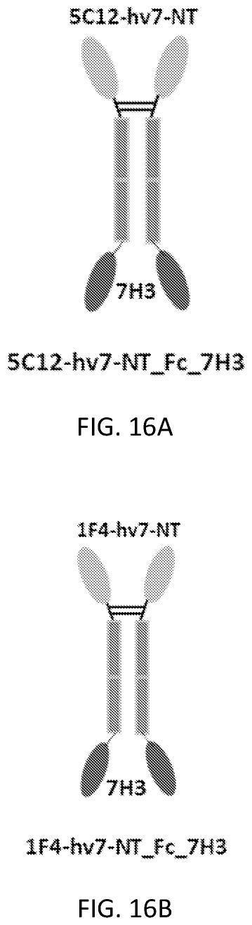

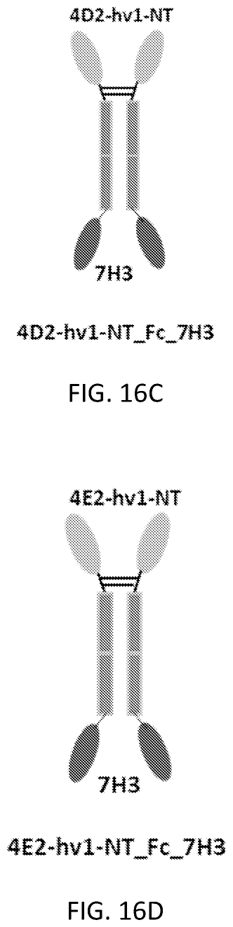

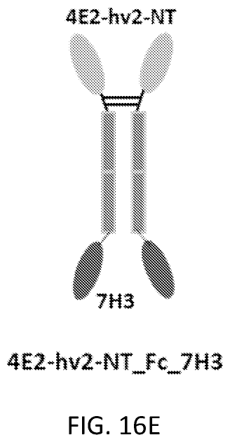

The format of anti-PD-L1_Fc_anti-VEGF can retain the potency for both arms against PD-L1 or VEGF, and has the highest monomer ratio in SEC (FIG. 14 and the Table 24). Thus, we used this format to construct all lead PD-L1 clones for BsAbs (FIG. 16). Sequences are shown in the four tables below.

TABLE 25
sequence of 5C12-hv7-NT_Fc_7H3 (SEQ ID NO: 89)

| | | Linker2 | 7H3 |
|---|---|---|---|
| 5C12-hv7-NT | Hinge+CH2+CH3 (IgG1) | (SEQ ID | (SEQ ID |
| (SEQ ID NO: 60) | (SEQ ID NO: 204) | NO: 203) | NO: 81) |
| QVQLVESGGGLVKPGGS | EPKSSDKTHTCPPCPAPELLGGPSVFLFPPK | GSGGS | QVQLVESGGGVVQP |
| LRLSCAASGFSASDVVM | PKDTLMISRTPEVTCVVVDVSHEDPEVKFNW | GGSGG | GRSLRLSCAASGFT |
| AWYRQAPGKQREFVSNI | YVDGVEVHNAKTKPREEQYNSTYRVVSVLTV | SGGSG | FGPYAMHWVRQAPG |
| AARGSTDYADSVKGRFT | LHQDWLNGKEYKCKVSNKALPAPIEKTISKA | | KERELVAAIWPSGS |
| ISRDNAKNSLYLQMNSL | KGQPREPQVYTLPPSREEMTKNQVSLTCLVK | | ITYYADSVKGRFTI |
| RAEDTAVYYCNALVWTG | GFYPSDIAVEWESNGQPENNYKTTPPVLDSD | | SRDNSKNTLYLQMN |
| SSYNNWGQGTQVTVSS | GSFFLYSKLTVDKSRWQQGNVFSCSVMHEAL | | SLRAEDTAVYYCAF |
| | HNHYTQKSLSLSPGK | | GSPTGSITYWGQGT |
| | | | LVTVSS |

TABLE 26
sequence of 1F4-hv7-NT_Fc_7H3 (SEQ ID NO: 90)

| | | Linker2 | 7H3 |
|---|---|---|---|
| 1F4-hv7-NT | Hinge+CH2+CH3(IgG1) | (SEQ ID | (SEQ ID |
| (SEQ ID NO: 68) | (SEQ ID NO: 204) | NO: 203) | NO: 81) |
| QVQLVESGGGLVKPGGS | EPKSSDKTHTCPPCPAPELLGGPSVFLFPPK | GSGGS | QVQLVESGGGVVQ |
| LRLSCAASGFSASDVVM | PKDTLMISRTPEVTCVVVDVSHEDPEVKFNW | GGSGG | PGRSLRLSCAASG |
| AWYRQAPGKQREFVANI | YVDGVEVHNAKTKPREEQYNSTYRVVSVLTV | SGGSG | FTFGPYAMHWVRQ |
| AASSTTRYADSVKGRFT | LHQDWLNGKEYKCKVSNKALPAPIEKTISKA | | APGKERELVAAIW |
| ISRDNAKNSLYLQMNSL | KGQPREPQVYTLPPSREEMTKNQVSLTCLVK | | PSGSITYYADSVK |
| RAEDTAVYYCNALVWTG | GFYPSDIAVEWESNGQPENNYKTTPPVLDSD | | GRFTISRDNSKNT |
| SSYNNWGQGTQVTVSS | GSFFLYSKLTVDKSRWQQGNVFSCSVMHEAL | | LYLQMNSLRAEDT |
| | HNHYTQKSLSLSPGK | | AVYYCAFGSPTGS |
| | | | ITYWGQGTLVTVS |
| | | | S |

TABLE 27
sequence of 4D2-hv1-NT_Fc_7H3 (SEQ ID NO: 91)

| | | Linker2 | 7H3 |
|---|---|---|---|
| 4D2-hv1-NT | Hinge+CH2+CH3 (IgG1) | (SEQ ID | (SEQ ID |
| (SEQ ID NO: 74) | (SEQ ID NO: 204) | NO: 203) | NO: 81) |
| QVQLVESGGGLVKPGG | EPKSSDKTHTCPPCPAPELLGGPSVFLFPP | GSGGS | QVQLVESGGGVVQPG |
| SLRLSCAASGFSASDV | KPKDTLMISRTPEVTCVVVDVSHEDPEVKF | GGSGG | RSLRLSCAASGFTFG |
| VMAWYRQAPGKQREFV | NWYVDGVEVHNAKTKPREEQYNSTYRVVSV | SGGSG | PYAMHWVRQAPGKER |
| ANLGASSTTRYADSVK | LTVLHQDWLNGKEYKCKVSNKALPAPIEKT | | ELVAAIWPSGSITYY |
| GRFTISRDNAKNSLYL | ISKAKGQPREPQVYTLPPSREEMTKNQVSL | | ADSVKGRFTISRDNS |
| QMNSLRAEDTAVYYCN | TCLVKGFYPSDIAVEWESNGQPENNYKTTP | | KNTLYLQMNSLRAED |
| ALVWTGSSYNNWGQGT | PVLDSDGSFFLYSKLTVDKSRWQQGNVFSC | | TAVYYCAFGSPTGSI |
| QVTVSS | SVMHEALHNHYTQKSLSLSPGK | | TYWGQGTLVTVSS |

TABLE 28
sequence of 4E2-hv1-NT_Fc_7H3 (SEQ ID NO: 92)

| | | Linker2 | 7H3 |
|---|---|---|---|
| 4E2-hv1-NT | Hinge+CH2+CH3 | (SEQ ID | (SEQ ID |
| (SEQ ID NO: 78) | (SEQ ID NO: 204) | NO: 203) | NO: 81) |
| EVQLVESGGGLVKPG | EPKSSDKTHTCPPCPAPELLGGPSVFLFP | GSGGSGG | QVQLVESGGGVVQPGRS |
| GSLRLSCAASGFSAG | PKPKDTLMISRTPEVTCVVVDVSHEDPEV | SGGSGGS | LRLSCAASGFTFGPYAM |
| DVVMAWYRQAPGKQR | KFNWYVDGVEVHNAKTKPREEQYNSTYRV | G | HWVRQAPGKERELVAAI |
| EFVANIAASSTTRYA | VSVLTVLHQDWLNGKEYKCKVSNKALPAP | | WPSGSITYYADSVKGRF |
| DSVKGRFTISRDNAK | IEKTISKAKGQPREPQVYTLPPSREEMTK | | TISRDNSKNTLYLQMNS |
| NSLYLQMNSLRAEDT | NQVSLTCLVKGFYPSDIAVEWESNGQPEN | | LRAEDTAVYYCAFGSPT |
| AVYYCNALVWTGSSY | NYKTTPPVLDSDGSFFLYSKLTVDKSRWQ | | GSITYWGQGTLVTVSS |
| NNWGQGTQVTVSS | QGNVFSCSVMHEALHNHYTQKSLSLSPGK | | |

TABLE 29
sequence of 4E2-hv2-NT_Fc_7H3 (SEQ ID NO: 93)

| | | Linker2 | 7H3 |
|---|---|---|---|
| 4E2-hv2-NT | Hinge+CH2+CH3 (IgG1) | (SEQ ID | (SEQ ID |
| (SEQ ID NO: 79) | (SEQ ID NO: 204) | NO: 203) | NO: 81) |
| EVQLVESGGGLVKPG | EPKSSDKTHTCPPCPAPELLGGPSVFLF | GSGGSGG | QVQLVESGGGVVQPGRSLR |
| GSLRLSCAASGFSAG | PPKPKDTLMISRTPEVTCVVVDVSHEDP | SGGSGGS | LSCAASGFTFGPYAMHWVR |
| DVVMAWYRQAPGKQR | EVKFNWYVDGVEVHNAKTKPREEQYNST | G | QAPGKERELVAAIWPSGSI |
| EFVSNIAASSTTRYA | YRVVSVLTVLHQDWLNGKEYKCKVSNKA | | TYYADSVKGRFTISRDNSK |
| DSVKGRFTISRDNAK | LPAPIEKTISKAKGQPREPQVYTLPPSR | | NTLYLQMNSLRAEDTAVYY |
| NSLYLQMNSLRAEDT | EEMTKNQVSLTCLVKGFYPSDIAVEWES | | CAFGSPTGSITYWGQGTLV |
| AVYYCNALVWTGSS | NGQPENNYKTTPPVLDSDGSFFLYSKLT | | TVSS |
| YNNWGQGTQVTVSS | VDKSRWQQGNVFSCSVMHEALHNHYTQK | | |
| | SLSLSPGK | | |

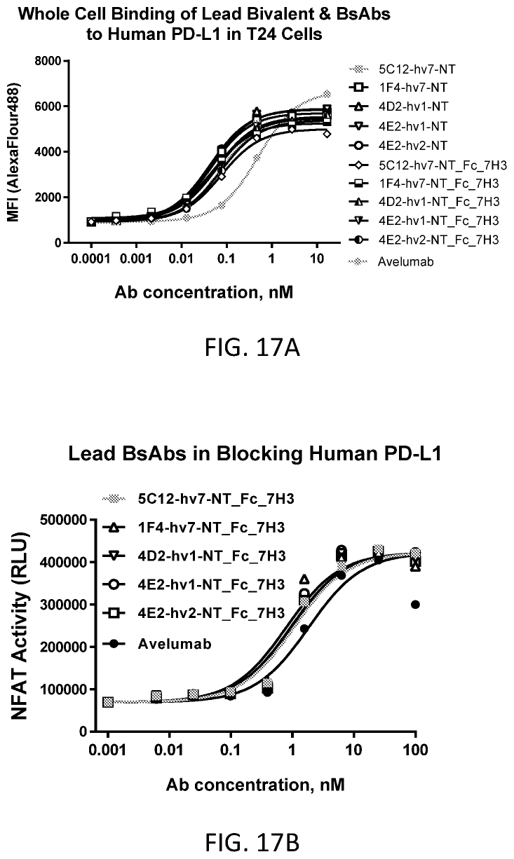

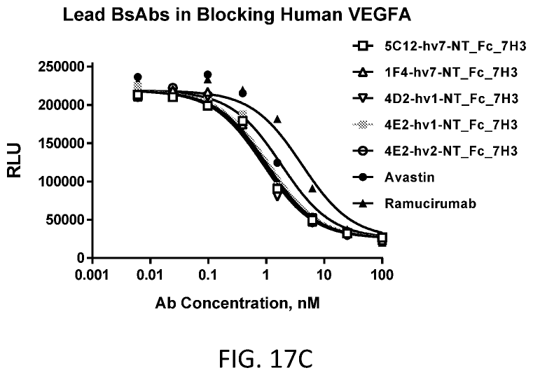

Binding abilities of these BsAbs and their parental monospecific anti-PD-L1 bivalent antibodies (all fused with human IgG1 Fc) to human PD-L1 expressed in T24 tumor cells were determined by whole cell binding assays, and results are shown in FIG. 17A. The potencies of these BsAbs and their parental monospecific anti-PD-L1 bivalent antibodies in blocking h-PD-L1 were also determined and results are shown in FIG. 17B. Compared to their parental monospecific, bivalent format, the binding abilities and potencies of BsAbs in blocking h-PD-L1 were mostly retained. The potencies in blocking h-VEGFA are shown in FIG. 17C and the table below.

TABLE 30
Whole cell binding ability to h-PD-L1 and
potency in blocking h-PD-L1 and h-VEGFA

| | | | EC50 (nM) in |
|---|---|---|---|
| | EC50 (nM) in WCB to | EC50 (nM) in | blocking h- |
| Clone Names | T24 cells | blocking h-PD-L1 | VEGFA |
| 5C12-hv7-NT_Fc | 0.083 | 0.94 | No |
| 4D2-hv1-NT_Fc | 0.043 | 1.02 | No |
| 1F4-hv7-NT_Fc | 0.047 | 1.04 | No |
| 4E2-hv1-NT_Fc | 0.048 | 0.76 | No |
| 4E2-hv2-NT_Fc | 0.047 | 0.90 | No |
| 5C12-hv7-NT-Fc-7H3 | 0.075 | 1.07 | 0.97 |
| (SEQ ID NO: 89) | | | |
| 1F4-hv7-NT_Fc_7H3 | 0.044 | 0.92 | 1.01 |
| (SEQ ID NO: 90) | | | |
| 4D2-hv1-NT_Fc_7H3 | 0.054 | 0.88 | 0.87 |
| (SEQ ID NO: 91) | | | |
| 4E2-hv1-NT_Fc_7H3 | 0.055 | 0.85 | 1.12 |
| (SEQ ID NO: 92) | | | |
| 4E2-hv2-NT_Fc_7H3 | 0.074 | 0.97 | 1.01 |
| (SEQ ID NO: 93) | | | |
| Avelumab | 0.501 | 1.73 | No |
| Avastin | No | No | 1.79 |
| Ramucirumab | No | No | 4.05 |

No = no activity

### Example 9: Construction of Surrogate Anti-PD-L1× Anti-VEGF BsAbs and Potencies of the BsAbs in Blocking Mouse PD-L1 and Mouse VEGFA

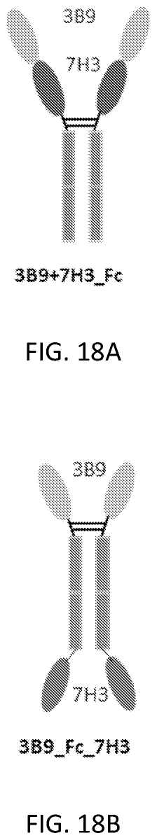

Because of lead anti-PD-L1 clones do not bind/block mouse PD-L1, clone 3B9 was selected as a surrogate PD-L1 blocker due to its potency in blocking mouse PD-L1 as shown in Example 3. First, we made a new bivalent 7H3-Fc clone by converting 7H3-IgG4-Fc to 7H3-IgG1-Fc, then we constructed the tandem format BsAb 3B9+7H3 Fc and the N- or C terminal format BsAb 3B9_Fc_7H3 (FIG. 18). Their sequences are shown in the two tables below.

TABLE 31
sequence of 3B9+7H3_Fc (SEQ ID NO: 94)

| 3B9 | Linker1 | 7H3 | |
|---|---|---|---|
| (SEQ ID | (SEQ ID | (SEQ ID | Hinge+CH2+CH3 (IgG1) |
| NO: 33) | NO: 202) | NO: 81) | (SEQ ID NO: 204) |
| QVQLVESGGGVVQP | GSGGSGGS | QVQLVESGGGVVQP | EPKSSDKTHTCPPCPAPELLGGPSVFLFPPKPK |
| GGSLRLSCAASGID | GGSG | GRSLRLSCAASGFT | TLMISRTPEVTCVVVDVSHEDPEVKFNWYVDGD |
| FSDTHMHWVRQAPG | | FGPYAMHWVRQAPG | VEVHNAKTKPREEQYNSTYRVVSVLTVLHQDWL |
| KQRELVAIITSGGF | | KERELVAAIWPSGS | NGKEYKCKVSNKALPAPIEKTISKAKGQPREPQ |
| TDYVDSVKGRFTIS | | ITYYADSVKGRFTI | VYTLPPSREEMTKNQVSLTCLVKGFYPSDIAVE |
| RDNSKNTLYLQMNS | | SRDNSKNTLYLQMN | WESNGQPENNYKTTPPVLDSDGSFFLYSKLTVD |
| LRAEDTAVYYCNAR | | SLRAEDTAVYYCAF | KSRWQQGNVFSCSVMHEALHNHYTQKSLSLSPG |
| TSTIYWGQGTQVTV | | GSPTGSITYWGQGT | K |
| SS | | LVTVSS | |

TABLE 32
sequence of 3B9_Fc_7H3 (SEQ ID NO: 95)

| 3B9 | | Linker2 | 7H3 |
|---|---|---|---|
| (SEQ ID | Hinge+CH2+CH3 (IgG1) | (SEQ ID | (SEQ ID |
| NO: 33) | (SEQ ID NO: 204) | NO: 203) | NO: 81) |
| QVQLVESGGGVVQP | EPKSSDKTHTCPPCPAPELLGGP | GSGGSGG | QVQLVESGGGVVQPGRSLRLSCAAS |
| GGSLRLSCAASGID | SVFLFPPKPKDTLMISRTPEVTC | SGGSGGS | GFTFGPYAMHWVRQAPGKERELVAA |
| FSDTHMHWVRQAPG | VVVDVSHEDPEVKFNWYVDGVEV | G | IWPSGSITYYADSVKGRFTISRDNS |
| KQRELVAIITSGGF | HNAKTKPREEQYNSTYRVVSVLT | | KNTLYLQMNSLRAEDTAVYYCAFGS |
| TDYVDSVKGRFTIS | VLHQDWLNGKEYKCKVSNKALPA | | PTGSITYWGQGTLVTVSS |
| RDNSKNTLYLQMNS | PIEKTISKAKGQPREPQVYTLPP | | |
| LRAEDTAVYYCNAR | SREEMTKNQVSLTCLVKGFYPSD | | |
| TSTIYWGQGTQVTV | IAVEWESNGQPENNYKTTPPVLD | | |
| SS | SDGSFFLYSKLTVDKSRWQQGNV | | |
| | FSCSVMHEALHNHYTQKSLSLSP | | |
| | GK | | |

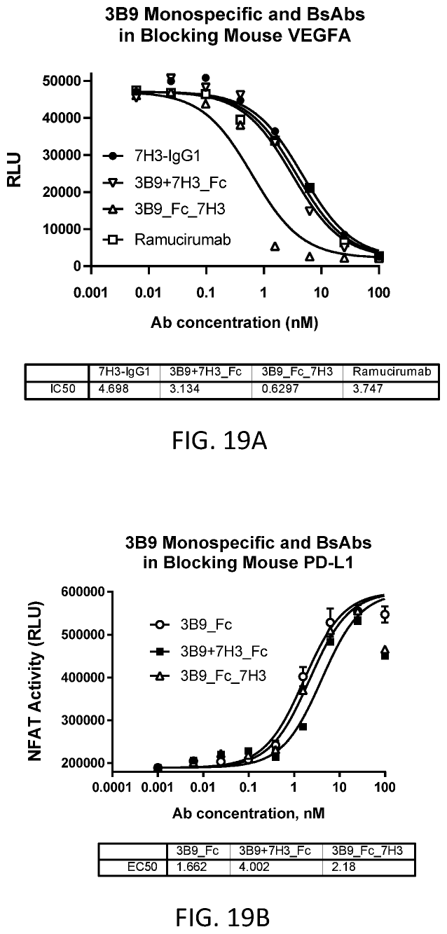

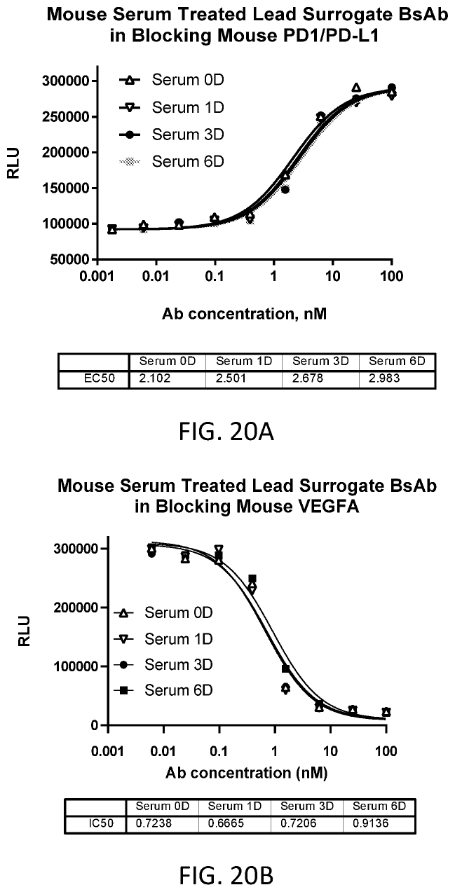

Blocking function of BsAbs in mouse VEGFA-mediated NFAT luciferase reporter activity was measured and results are shown in FIG. 19A. Tandem format BsAb retained the blocking function to VEGFA with a potency similar to bivalent parental clone 7H3 or reference antibody Ramucirumab. Interestingly when anti-VEGFA 7H3 was fused to the C terminal of the BsAb, the 3B9_Fc_7H3 showed significantly higher potency in blocking m-VEGFA compared to the bivalent 7H3-Fc parental clone, with a more than 7-fold increase in potency. The potency of BsAbs (both formats) in blocking mouse PD-L1 was also examined. As shown in FIG. 19B, both BsAbs blocked mouse PD-L1 activity with 3B9_Fc_7H3 being slightly more potent than 3B9+7H3_Fc, and showing a similar potency to parental clone bivalent 3B9. Based on the function of both arms of anti-PD-L1 and anti-VEGFA, 3B9 Fc 7H3 selected as a surrogate BsAb candidate for in vivo studies. The stability of 3B9 Fc_7H3 in mouse serum was tested by incubation of 3B9_Fc_7H3 with 66.6% mouse serum at 37 °C for different period of time. The function in blocking mouse PD-L1 and mouse VEGFA was examined. FIGS. 20A & 20B show that 3B9 Fc 7H3 is stable under mouse serum treatment at 37 °C for up to 6 days.

### Example 10: In Vivo Efficacy Study for Surrogate Anti-PD-L1× Anti-VEGF BsAb and Monospecific Anti-PD-L1 and Anti-VEGF Antibodies in MC38 Syngeneic Mouse Model

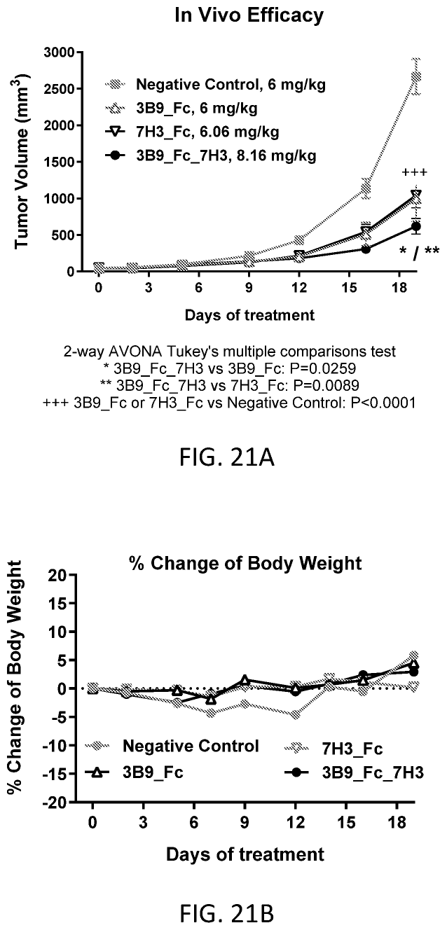

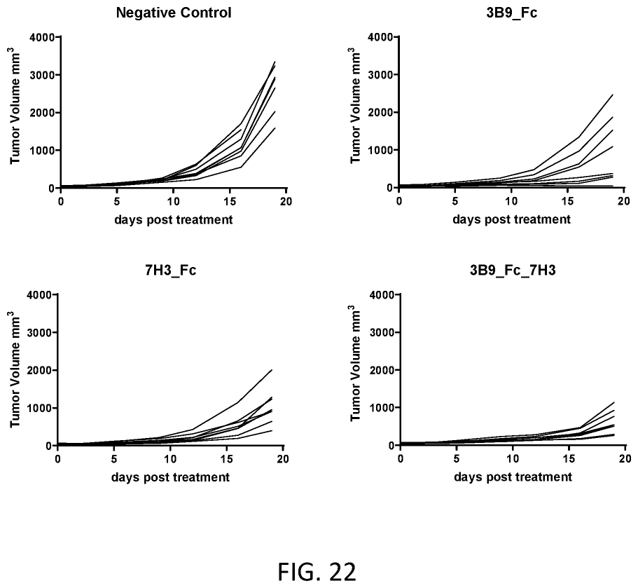

Using a C57BL/6N mouse model with MC38 murine colon cancer, efficacy of BsAb 3B9 Fc_7H3 was studied to compare the efficacies of monospecific antibody 3B9_Fc (anti-PD-L1) or 7H3_Fc (anti-VEGF). MC38 tumor cells were implanted one week before treatment. Treatment started when tumor reached about 46 mm³ volume, drugs were dosed intraperitoneally three times a week for 3 weeks with 6 mg/kg or equal molar amount as shown in FIG. 21, total 8 dosing were given. Both 3B9_Fc and 7H3_Fc significantly reduced tumor growth compared to negative control (inactive anti-PD-L1 variant 5F8-hv2) with tumor growth inhibition (TGI) of 62.7 and 60.9%, respectively at day 19 post-treatment initiation (FIG. 21A and the table below). BsAb 3B9_Fc_7H3 had better efficacy compared to monospecific antibodies with TGI reached 76.9% at day 19 post-treatment. Statistical analysis using two-way ANOVA Tukey's multiple comparisons test indicates that tumor inhibition of all three treatment groups were significant compared to negative control, with P<0.0001. Tumor inhibition of 3B9_Fc_7H3 was also significant when compared to TGI of either 3B9 Fc or 7H3_Fc, with P=0.0259 or 0.0089, respectively. Individual tumor volumes of each treatment group shown in FIG. 22. No significantly difference of body weight observed among mice from different study groups, indicating drugs were well-tolerated (FIG. 21 B).

TABLE 33
Efficacy summary in MC38 syngeneic model

| | | | | | | | % TGI |
|---|---|---|---|---|---|---|---|
| Groups | Treatment | N | TV (mm³)^{a} | P ^{b} | P ^{c} | P ^{d} | (Day 19) |
| 1 | Negative | 8 | 2666.2 ± 242.7 | — | — | — | — |
| | Control | | | | | | |
| 2 | 3B9_Fc | 8 | 994.5 ± 312.7 | <0.0001 | — | — | 62.70% |
| 3 | 7H3_Fc | 8 | 1042.0 ± 171.5 | <0.0001 | — | — | 60.92% |
| 4 | 3B9_Fc_7H3 | 8 | 616.9 ± 107.9 | <0.0001 | 0.0259 | 0.0089 | 76.86% |

^{a}Mean ± SEM
^{b} Compare to Negative Control by 2-way ANOVA Tukey's multiple comparisons test
^{c} Compare to 3B9_Fc by 2-way ANOVA Tukey's multiple comparisons test
^{d} Compare to 7H3_Fc by 2-way ANOVA Tukey's multiple comparisons test

### Example 11: Comparison Study of Lead BsAbs to Other Anti-PD-L1/PD1× Anti-VEGF Bi-Functional Drug Candidate IMM2510 or AK112 in Cell-Based Functional Assays

Currently there are two leading bi-functional drug candidates in clinical trials, IMM2510 & AK112. IMM2510 is a traditional anti-PD-L1 antibody fused with a VEGF Trap in the N-terminal of the heavy chain. VEGFTrap is a fusion protein which combines ligand-binding elements taken from extracellular components of human VEGF receptor I (the domain2 of VEGFR1) and human VEGF receptor II (the domain3 of VEGFRII). VEGF Trap binds its ligand VEGFA with a high affinity, thereby blocking the function of VEGFA. Currently IMM2510 is in a phase I clinical trial. AK112 is a BsAb, constructed using Avastin (Roche's anti-VEGF antibody) fused with a scFv (single-chain variable fragment) of an anti-PD1 antibody in the C-terminal of the heavy chain of Avastin. Currently AK112 is in a phase III clinical trial.

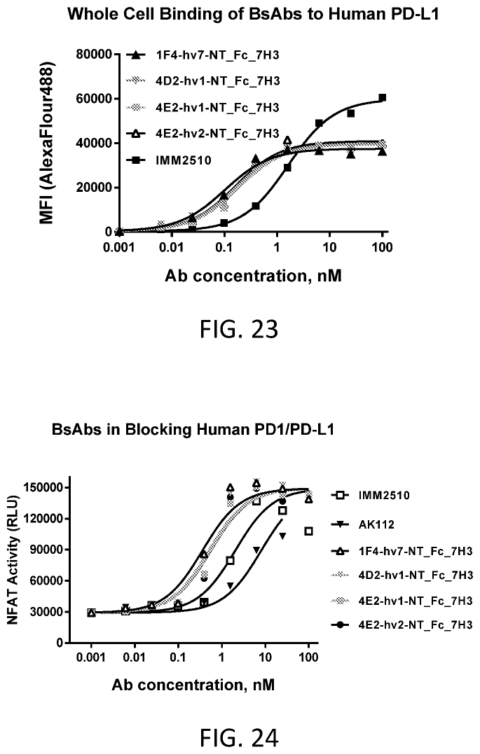

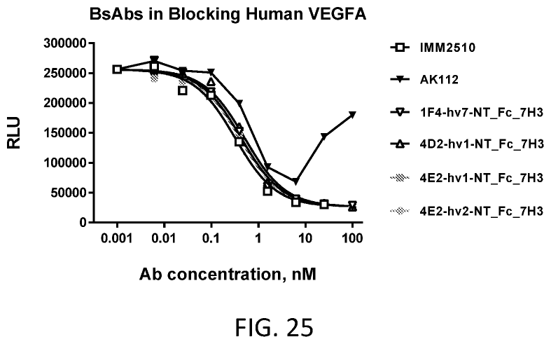

Comparison study by whole cell binding assay was performed using human PD-L1 stably transfected CHO cells (see FIG. 23 and Table 34). Our lead BsAbs all showed much higher binding ability compared to IMM2510, with EC50 about 10-fold lower than that of IMM2510. Whole cell binding assay of AK112 to PD1 stably transfected Jurkat cells was also performed and EC50 data is shown in Table 34. In human PD1/PD-L1 blockade assay, our lead BsAbs demonstrated more potent function in blocking PD1/PD-L1 compared to IMM2510 and AK112 (see FIG. 24 and Table 34), with EC50 in the range of 0.36-0.56 nM for our BsAbs. IMM2510 and AK112 showed EC50 of 2.04 nM and 8.08 nM, respectively. In a human VEGFA blockade assay, potencies of our BsAbs and IMM2510 are similar, although IMM2510 is slightly more potent. AK112 is weaker in blocking VEGFA and even gradually lost inhibitory function when the concentration increased to above 10 nM (FIG. 25).

SEC-HPLC was also performed to compare IMM2510 and AK112 to our lead BsAbs, as shown in Table 34. Our BsAbs have more than 98% monomer in SEC-HPLC assay, however IMM2510 only has 92.9% monomer with 6.8% degraded fragments shown as low molecular weight (LMW) species. AK112 is the worst molecule in stability, only has 65.8% monomer and has 21.2% aggregates shown as high molecular weight (HMW) species, and 12.9% degraded fragments shown as LMW species. All protein samples were purified by the same purification method using Protein A resin.

Taken together, our lead anti-PD-L1× anti-VEGF BsAbs are more potent in both arms against either human PD1/PD-L1 or VEGFA than AK112, in cell-based functional assays. Also our lead BsAbs are more potent than IMM2510 in blocking human PD-L1, and equally potent with IMM2510 in blocking human VEGFA. In addition, our lead BsAbs have better SEC profiles than both IMM2510 and AK112, are more stable molecules with much better developability as medicines for cancer patients.

TABLE 34
Comparison of lead BsAbs to reference BsAbs in whole cell binding,
human PD1/PD-L1 blockade, human VEGFA blockade, and SEC-HPLC assays

| | EC50 (nM) in | EC50 (nM) in | EC50 (nM) in | EC50 (nM) in | SEC-HPLC | | |
|---|---|---|---|---|---|---|---|
| | WCB to PD1 | WCB to PD-L1 | blocking h- | blocking h- | % HMW | | % LMW |
| Clones | stable cells | stable cells | PD1/PDL1 | VEGFA | species | % Monomer | species |
| 1F4-hv7-NT_Fc_7H3 | No | 0.10 | 0.36 | 0.43 | 0.40 | 98.40 | 1.20 |
| 4D2-hv1-NT_Fc_7H3 | No | 0.16 | 0.50 | 0.51 | 0.60 | 98.90 | 0.50 |
| 4E2-hv1-NT_Fc_7H3 | No | 0.19 | 0.55 | 0.40 | 0.00 | 99.20 | 0.80 |
| 4E2-hv2-NT_Fc_7H3 | No | 0.16 | 0.56 | 0.41 | 0.60 | 98.00 | 1.40 |
| IMM2510 | No | 1.63 | 2.04 | 0.32 | 0.40 | 92.80 | 6.80 |
| AK112 | 9.47 | No | 8.08 | 1.18* | 21.20 | 65.90 | 12.9 |

No = No activity.
*Not a good nonlinear regression fitting as shown in FIG. 25.

### Example 12: In Vivo Efficacy Study for Lead Anti-PD-L1× Anti-VEGF BsAb Using MC38-hPD-L1 Murine Colon Cancer Cell Model in C57BL/6N Syngeneic Mice

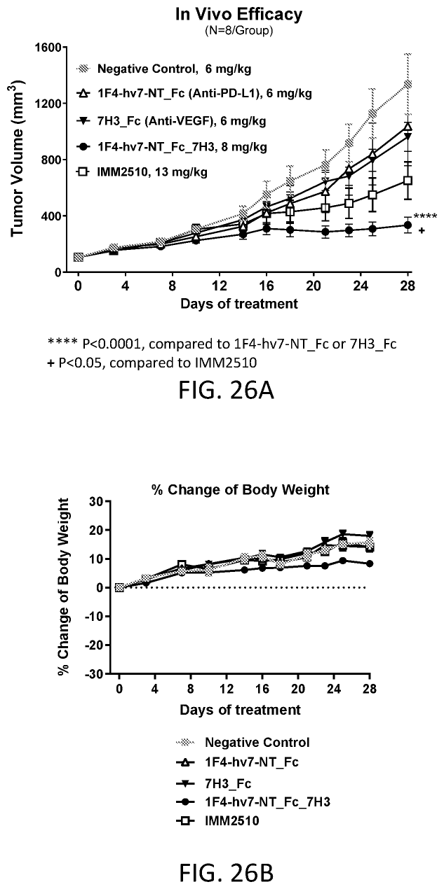

MC38-hPD-L1 (MC38CD274) is a murine colon carcinoma cell line (MC38), engineered to express human PD-L1 gene (CD274) by knocking out mouse PD-L1 gene and knocking in human PD-L1 (hPD-L1) transgene into the mouse PD-L1 locus. Using C57BL/6N mice with MC38-hPD-L1 murine colon cancer model, the efficacy of anti-PD-L1× anti-VEGF BsAb 1F4-hv7-NT_Fc_7H3 was evaluated and compared to the efficacies of monospecific antibodies 1F4-hv7-NT_Fc (Anti-PD-L1), 7H3_Fc (anti-VEGF), and a reference BsAb, IMM2510. MC38-hPD-L1 tumor cells (1×10⁶) were implanted one week before treatment. Treatment started when tumor volumes reached about 105 mm³ on average. Drugs were dosed intraperitoneally three times a week for 3 weeks at 6 mg/kg or equal molar amounts. Statistical analysis of tumor volumes (by 2-way ANOVA with Tukey's multiple comparisons test) showed that all treatment groups significantly inhibited tumor growth compared to the negative control (inactive anti-PD-L1 variant 5F8-hv2) (Table 35). Both monospecific antibodies 1F4-hv7-NT_Fc and 7H3_Fc significantly reduced tumor growth compared to the negative control with tumor growth inhibition (TGI) of 24.25% and 30.33%, respectively on day 28 post-treatment initiation (FIG. 26A & Table 35). BsAb 1F4-hv7-NT_Fc_7H3 showed a much stronger efficacy compared to monospecific antibodies, with a TGI of 81.32% on day 28 post-treatment. Significantly increased tumor suppression was also observed for BsAb 1F4-hv7-NT_Fc_7H3 compared to the reference BsAb IMM2510 with P<0.013 (TGI: 81.32% vs 55.62%).

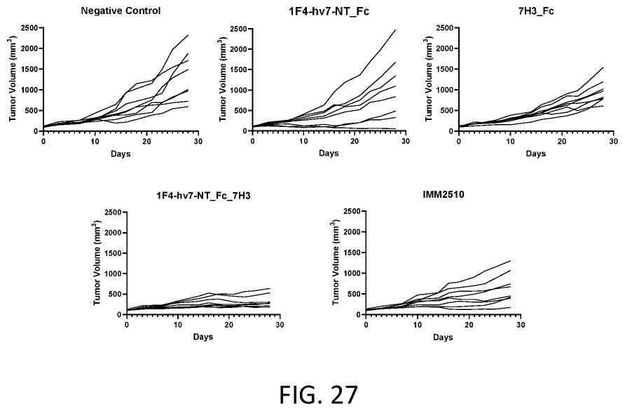

No significant change of body weight was observed among mice from different study groups, indicating drugs were well-tolerated (FIG. 26B). Individual tumor volume data of each treatment group are shown in FIG. 27.

TABLE 35
Efficacy summary of M38-PD-L1 tumor model in C57BL/6N syngeneic mice

| | | Tumor Volume | | | | % TGI |
|---|---|---|---|---|---|---|
| Treatment | N | (mm³)^{a} | P^{b} | P^{c} | P^{d} | (Day 28) |
| Negative Control | 8 | 1335.76 ± 206.93 | — | | | — |
| 1F4-hv7-NT_Fc | 8 | 1037.48 ± 245.63 | 0.0070 | | | 24.25 |
| 7H3_Fc | 8 | 962.71 ± 72.63 | 0.0114 | | | 30.33 |
| 1F4-hv7- | 8 | 335.53 ± 45.95 | <0.0001 | <0.0001 | 0.0136 | 81.32 |
| NT_Fc_7H3 | | | | | | |
| IMM2510 | 8 | 651.58 ± 114.78 | <0.0001 | | | 55.62 |

^{a}Mean ± SEM
^{b}Compared to Negative Control
^{c}Compared to 1F4-hv7-NT_Fc or 7H3_Fc.
^{d}Compared to IMM2510.
Statistical analysis performed by 2-way ANOVA with Tukey's multiple comparisons test.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen-binding fragment thereof that binds to PD-L1
(Programmed death-ligand 1), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 96, 97, and 98, respectively;
(2) the selected VHH CDRs 1, 2, 3 amino acid sequences are set forth in SEQ ID NOs: 99, 100, and 101, respectively;
(3) the selected VHH CDRs 1, 2, 3 amino acid sequences are set forth in SEQ ID NOs: 102, 103 and 104, respectively;
(4) the selected VHH CDRs 1, 2, 3 amino acid sequences are set forth in SEQ ID NOs: 105, 106, and 107, respectively;
(5) the selected VHH CDRs 1, 2, 3 amino acid sequences are set forth in SEQ ID NOs: 108, 109, and 110, respectively;
(6) the selected VHH CDRs 1, 2, 3 amino acid sequences are set forth in SEQ ID NOs: 111, 112, and 113, respectively;
(7) the selected VHH CDRs 1, 2, 3 amino acid sequences are set forth in SEQ ID NOs: 114, 115, and 116, respectively;
(8) the selected VHH CDRs 1, 2, 3 amino acid sequences are set forth in SEQ ID NOs: 117, 118, and 119, respectively;
(9) the selected VHH CDRs 1, 2, 3 amino acid sequences are set forth in SEQ ID NOs: 120, 121, and 122, respectively;
(10) the selected VHH CDRs 1, 2, 3 amino acid sequences are set forth in SEQ ID NOs: 123, 124, and 125, respectively;
(11) the selected VHH CDRs 1, 2, 3 amino acid sequences are set forth in SEQ ID NOs: 126, 127, and 128, respectively;
(12) the selected VHH CDRs 1, 2, 3 amino acid sequences are set forth in SEQ ID NOs: 129, 130, and 131, respectively;
(13) the selected VHH CDRs 1, 2, 3 amino acid sequences are set forth in SEQ ID NOs: 132, 133, and 134, respectively;
(14) the selected VHH CDRs 1, 2, 3 amino acid sequences are set forth in SEQ ID NOs: 135, 136, and 137, respectively;
(15) the selected VHH CDRs 1, 2, 3 amino acid sequences are set forth in SEQ ID NOs: 138, 139, and 140, respectively;
(16) the selected VHH CDRs 1, 2, 3 amino acid sequences are set forth in SEQ ID NOs: 141, 142, and 143, respectively;
(17) the selected VHH CDRs 1, 2, 3 amino acid sequences are set forth in SEQ ID NOs: 144, 145, and 146, respectively;
(18) the selected VHH CDRs 1, 2, 3 amino acid sequences are set forth in SEQ ID NOs: 147, 148, and 149, respectively;
(19) the selected VHH CDRs 1, 2, 3 amino acid sequences are set forth in SEQ ID NOs: 150, 151, and 152, respectively;
(20) the selected VHH CDRs 1, 2, 3 amino acid sequences are set forth in SEQ ID NOs: 153, 154, and 155, respectively;
(21) the selected VHH CDRs 1, 2, 3 amino acid sequences are set forth in SEQ ID NOs: 156, 157, and 158, respectively;
(22) the selected VHH CDRs 1, 2, 3 amino acid sequences are set forth in SEQ ID NOs: 159, 160, and 161, respectively;
(23) the selected VHH CDRs 1, 2, 3 amino acid sequences are set forth in SEQ ID NOs: 162, 163, and 164, respectively;
(24) the selected VHH CDRs 1, 2, 3 amino acid sequences are set forth in SEQ ID NOs: 165, 166, and 167, respectively;
(25) the selected VHH CDRs 1, 2, 3 amino acid sequences are set forth in SEQ ID NOs: 168, 169, and 170, respectively;
(26) the selected VHH CDRs 1, 2, 3 amino acid sequences are set forth in SEQ ID NOs: 171, 172, and 173, respectively;
(27) the selected VHH CDRs 1, 2, 3 amino acid sequences are set forth in SEQ ID NOs: 174, 175, and 176, respectively;
(28) the selected VHH CDRs 1, 2, 3 amino acid sequences are set forth in SEQ ID NOs: 177, 178, and 179, respectively;
(29) the selected VHH CDRs 1, 2, 3 amino acid sequences are set forth in SEQ ID NOs: 180, 181, and 182, respectively; and
(30) the selected VHH CDRs 1, 2, 3 amino acid sequences are set forth in SEQ ID NOs: 183, 184, and 185, respectively.

2. The antibody or antigen-binding fragment thereof of claim 1, wherein the VHH comprises CDRs 1, 2, 3 with the amino acid sequences set forth in SEQ ID NOs: 183, 184, and 185, respectively.

3. The antibody or antigen-binding fragment thereof of claim 1, wherein the VHH comprises CDRs 1, 2, 3 with the amino acid sequences set forth in SEQ ID NOs: 171, 172, and 173, respectively.

4. The antibody or antigen-binding fragment thereof of claim 1, wherein the VHH comprises CDRs 1, 2, 3 with the amino acid sequences set forth in SEQ ID NOs: 177, 178, and 179, respectively.

5. The antibody or antigen-binding fragment thereof of claim 1, wherein the VHH comprises CDRs 1, 2, 3 with the amino acid sequences set forth in SEQ ID NOs: 180, 181, and 182, respectively.

6. The antibody or antigen-binding fragment thereof of claim 1, wherein the VHH comprises CDRs 1, 2, 3 with the amino acid sequences set forth in SEQ ID NOs: 132, 133, and 134, respectively.

7. The antibody or antigen-binding fragment thereof of claim 1, comprising a heavy-chain antibody variable domain (VHH) comprising an amino acid sequence that is at least 80% identical to a selected VHH sequence, wherein the selected VHH sequence is selected from the group consisting of SEQ ID NOs: 1-80.

8.-18. (canceled)

19. An antibody or antigen-binding fragment thereof that cross-competes with the antibody or antigen-binding fragment thereof of claim 1.

20. An antibody or antigen-binding fragment thereof that binds to VEGF (Vascular endothelial growth factor), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 186, 187, and 188, respectively;
(2) the selected VHH CDRs 1, 2, 3 amino acid sequences are set forth in SEQ ID NOs: 189, 190, and 191, respectively;
(3) the selected VHH CDRs 1, 2, 3 amino acid sequences are set forth in SEQ ID NOs: 192, 193, and 194, respectively; and
(4) the selected VHH CDRs 1, 2, 3 amino acid sequences are set forth in SEQ ID NOs: 195, 196, and 197, respectively.

21.-35. (canceled)

36. The antibody or antigen-binding fragment thereof of claim 1, comprising a first VHH (VHH1) that specifically binds to VEGF, a second VHH (VHH2) that specifically binds to PD-L1, a third VHH (VHH3) that specifically binds to VEGF, and a fourth VHH (VHH4) that specifically binds to PD-L1.

37. (canceled)

38. The multi-specific antibody or antigen-binding fragment thereof of claim 36, wherein the VHH1 and/or the VHH3 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 28.

39. (canceled)

40. The multi-specific antibody or antigen-binding fragment thereof of claim 36, wherein the VHH2 and/or the VHH4 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 29.

41.-44. (canceled)

45. A polypeptide complex comprising the antibody or antigen-binding fragment thereof of claim 1, comprising
(a) a first polypeptide comprising from N-terminus to C-terminus: a first heavy-chain antibody variable domain (VHH1), a first hinge region, a first Fc region, and a second VHH (VHH2); and
(b) a second polypeptide comprising from N-terminus to C-terminus: a third VHH (VHH3), a second hinge region, a second Fc region, and a fourth VHH (VHH4),
wherein the VHH1 and the VHH3 specifically bind to PD-L1, and the VHH2 and the VHH4 specifically bind to VEGF.

46.-68. (canceled)

69. An antibody or antigen-binding fragment thereof that binds to PD-L1
(Programmed death-ligand 1), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence, wherein the selected VHH CDRs 1, 2, and 3 amino acid sequences are one of the following:
(1) the selected VHH CDR3 is NARX₁TTIY (SEQ ID NO: 198), wherein X₁=K, T, or R; the selected VHH CDR1 is selected from the group consisting of SEQ ID NOS: 96, 99, 102, 105, 108, 111, 126 and 129; and the selected VHH CDR2 is selected from the group consisting of SEQ ID NOS: 97, 100, 103, 106, 109, 112, 127 and 130;
(2) the selected VHH CDR3 is NALVWX₂GSSYNN (SEQ ID NO: 199), wherein X₂=Q, T, S, or N; the selected VHH CDR1 is selected from the group consisting of SEQ ID NOS: 114, 117, 120, 123, 168, 171, 174, 177, 180, and 183; and the selected VHH CDR2 is selected from the group consisting of SEQ ID NOS: 115, 118, 121, 124, 169, 172, 175, 178, 181, and 184; and
(3) the selected VHH CDR3 is selected from the group consisting of SEQ ID NOS: 134, 137, 140, 143, 146, 149, 152, 155, 158, 161, 164, and 167; the selected VHH CDR1 is selected from the group consisting of SEQ ID NOS: 132, 135, 138, 141, 144, 147, 150, 153, 156, 159, 162, and 165; and the selected VHH CDR2 is selected from the group consisting of SEQ ID NOS: 133, 136, 139, 142, 145, 148, 151, 154, 157, 160, 163, and 166.

70. (canceled)

71. A nucleic acid comprising a polynucleotide encoding the antibody or antigen-binding fragment thereof of claim 1.

72.-75. (canceled)

76. A cell comprising one or more of the nucleic acid of claim 71.

77. A method of producing an antibody or an antigen-binding fragment thereof, the method comprising
(a) culturing the cell of claim 76 under conditions sufficient for the cell to produce the antibody or the antigen-binding fragment; and
(b) collecting the antibody or the antigen-binding fragment produced by the cell.

78. An antibody-drug conjugate comprising the antibody or antigen-binding fragment thereof of claim 1, covalently bound to a therapeutic agent.

79. (canceled)

80. A method of treating a subject having cancer, the method comprising administering a therapeutically effective amount of a composition comprising the antibody or antigen-binding fragment thereof of claim 1, to the subject.

81.-86. (canceled)

87. A pharmaceutical composition comprising the antibody or antigen-binding fragment thereof of claim 1, and a pharmaceutically acceptable carrier.
